SEQUENCE LISTING (55 sequences)

SEQ ID NO: 1 - protein, 14 aa, Artificial Sequence
  source: /note="Description of Artificial Sequence: Synthetic peptide"
  MOD_RES (4)..(4): (R)-2-(7'-octenyl)alanine
  MOD_RES (11)..(11): (S)-2-(4'-pentenyl)alanine
LTFXEYWAQLXSAA

SEQ ID NO: 2 - protein, 17 aa, Artificial Sequence
  source: /note="Description of Artificial Sequence: Synthetic peptide"
ATVNVLAWLYAAVINGD

SEQ ID NO: 3 - protein, 17 aa, Artificial Sequence
  source: /note="Description of Artificial Sequence: Synthetic peptide"
  MOD_RES (11)..(11): (S)-2-(4'-pentenyl)alanine
  MOD_RES (15)..(15): (S)-2-(4'-pentenyl)alanine
ATVNVLAWLYXAVIXGD

SEQ ID NO: 4 - protein, 19 aa, Artificial Sequence
  source: /note="Description of Artificial Sequence: Synthetic peptide"
  MOD_RES (7)..(7): Norleucine
  MOD_RES (8)..(8): (S)-2-(4'-pentenyl)alanine
  MOD_RES (12)..(12): (S)-2-(4'-pentenyl)alanine
ANLNAGXXLGSXAATVELQ

SEQ ID NO: 5 - protein, 19 aa, Artificial Sequence
  source: /note="Description of Artificial Sequence: Synthetic peptide"
  MOD_RES (7)..(7): Norleucine
  MOD_RES (8)..(8): (S)-2-(4'-pentenyl)alanine
  MOD_RES (12)..(12): (S)-2-(4'-pentenyl)alanine
ANLNRGXXLGSXAATVRLQ

SEQ ID NO: 6 - protein, 7 aa, Homo sapiens
DQIVPEY

SEQ ID NO: 7 - protein, 14 aa, Artificial Sequence
  source: /note="Description of Artificial Sequence: Synthetic peptide"
  MOD_RES (4)..(4): (R)-2-(7'-octenyl)alanine
  MOD_RES (10)..(10): Cyclobutylalanine
  MOD_RES (11)..(11): (S)-2-(4'-pentenyl)alanine
LTFXEYWAQXXSAA

SEQ ID NO: 8 - protein, 5 aa, Artificial Sequence
  source: /note="Description of Artificial Sequence: Synthetic peptide"
  MOD_RES (2)..(4): Any amino acid
  source: /note="See specification as filed for detailed description of substitutions and preferred embodiments"
GXXXG

SEQ ID NO: 9 - protein, 16 aa, Severe acute respiratory syndrome coronavirus 2
TVNVLAWLYAAVINGD

SEQ ID NO: 10 - protein, 18 aa, Severe acute respiratory syndrome coronavirus 2
NLNRGMVLGSLAATVRLQ

SEQ ID NO: 11 - protein, 16 aa, Homo sapiens
LSQETFSDLWKLLPEN

SEQ ID NO: 12 - protein, 14 aa, Unknown
  source: /note="Description of Unknown: E3 ligase peptide"
LAPAAGDTIISLDF

SEQ ID NO: 13 - protein, 13 aa, Unknown
  source: /note="Description of Unknown: E3 ligase peptide"
LAPYIPMDDDFQL

SEQ ID NO: 14 - protein, 9 aa, Artificial Sequence
  source: /note="Description of Artificial Sequence: Synthetic peptide"
  MOD_RES (2)..(2): Any amino acid except Pro
  MOD_RES (3)..(3): Any amino acid except Pro
  MOD_RES (4)..(4): Any amino acid except Pro
  MOD_RES (6)..(6): Any amino acid except Pro
  MOD_RES (7)..(7): Any amino acid except Pro
  MOD_RES (8)..(8): Any amino acid except Pro
  SITE (6)..(8): /note="This region may encompass 2-3 residues"
  VARIANT (9)..(9): /replace="Ile" or "Leu"
  SITE (1)..(9): /note="Variant residues given in the sequence have no preference with respect to those in the annotations for variant positions"
FXXXWXXXV

SEQ ID NO: 15 - protein, 14 aa, Artificial Sequence
  source: /note="Description of Artificial Sequence: Synthetic peptide"
  VARIANT (1)..(1): /replace="Leu"
  MOD_RES (4)..(4): Any amino acid
  MOD_RES (5)..(5): Any amino acid
  MOD_RES (6)..(6): Any amino acid
  MOD_RES (7)..(7): Any amino acid
  MOD_RES (8)..(8): Any amino acid
  MOD_RES (9)..(9): Any amino acid
  MOD_RES (10)..(10): Any amino acid
  MOD_RES (11)..(11): Any amino acid
  SITE (4)..(11): /note="This region may encompass 6-8 residues"
  VARIANT (12)..(12): /replace="Leu" or "Ile" or "Val" or "Met"
  MOD_RES (13)..(13): Any amino acid
  VARIANT (14)..(14): /replace="Leu" or "Ile" or "Val" or "Met"
  SITE (1)..(14): /note="Variant residues given in the sequence have no preference with respect to those in the annotations for variant positions"
IAPXXXXXXXXFXF

SEQ ID NO: 16 - protein, 4 aa, Artificial Sequence
  source: /note="Description of Artificial Sequence: Synthetic peptide"
GGGS

SEQ ID NO: 17 - protein, 4 aa, Artificial Sequence
  source: /note="Description of Artificial Sequence: Synthetic peptide"
SGGG

SEQ ID NO: 18 - protein, 5 aa, Artificial Sequence
  source: /note="Description of Artificial Sequence: Synthetic peptide"
GGGGS

SEQ ID NO: 19 - protein, 5 aa, Artificial Sequence
  source: /note="Description of Artificial Sequence: Synthetic peptide"
SGGGG

SEQ ID NO: 20 - protein, 6 aa, Artificial Sequence
  source: /note="Description of Artificial Sequence: Synthetic peptide"
GGGGGS

SEQ ID NO: 21 - protein, 6 aa, Artificial Sequence
  source: /note="Description of Artificial Sequence: Synthetic peptide"
SGGGGG

SEQ ID NO: 22 - protein, 7 aa, Artificial Sequence
  source: /note="Description of Artificial Sequence: Synthetic peptide"
GGGGGGS

SEQ ID NO: 23 - protein, 7 aa, Artificial Sequence
  source: /note="Description of Artificial Sequence: Synthetic peptide"
SGGGGGG

SEQ ID NO: 24 - protein, 5 aa, Artificial Sequence
  source: /note="Description of Artificial Sequence: Synthetic peptide"
  source: /note="See specification as filed for detailed description of substitutions and preferred embodiments"
GGGGS

SEQ ID NO: 25 - protein, 5 aa, Artificial Sequence
  source: /note="Description of Artificial Sequence: Synthetic peptide"
  source: /note="See specification as filed for detailed description of substitutions and preferred embodiments"
SGGGG

SEQ ID NO: 26 - protein, 15 aa, Artificial Sequence
  source: /note="Description of Artificial Sequence: Synthetic peptide"
GGGGSGGGGSGGGGS

SEQ ID NO: 27 - protein, 25 aa, Artificial Sequence
  source: /note="Description of Artificial Sequence: Synthetic peptide"
GGGGSGGGGSGGGGSGGGGSGGGGS

SEQ ID NO: 28 - protein, 25 aa, Artificial Sequence
  source: /note="Description of Artificial Sequence: Synthetic peptide"
  MOD_RES (4)..(5): Any amino acid
  MOD_RES (9)..(10): Any amino acid
  MOD_RES (14)..(15): Any amino acid
  MOD_RES (19)..(20): Any amino acid
  SITE (1)..(20): /note="This region may encompass 0-4 'Gly Gly Gly Xaa Xaa' repeating units"
  source: /note="See specification as filed for detailed description of substitutions and preferred embodiments"
GGGXXGGGXXGGGXXGGGXXGGGGS

SEQ ID NO: 29 - protein, 25 aa, Artificial Sequence
  source: /note="Description of Artificial Sequence: Synthetic peptide"
  MOD_RES (6)..(6): Any amino acid
  MOD_RES (11)..(11): Any amino acid
  MOD_RES (16)..(16): Any amino acid
  MOD_RES (21)..(21): Any amino acid
  SITE (6)..(25): /note="This region may encompass 0-4 'Xaa Gly Gly Gly Ser' repeating units"
  source: /note="See specification as filed for detailed description of substitutions and preferred embodiments"
GGGGSXGGGSXGGGSXGGGSXGGGS

SEQ ID NO: 30 - protein, 25 aa, Artificial Sequence
  source: /note="Description of Artificial Sequence: Synthetic peptide"
  SITE (1)..(20): /note="This region may encompass 0-4 'Gly Gly Gly Pro Ser' repeating units"
GGGPSGGGPSGGGPSGGGPSGGGGS

SEQ ID NO: 31 - protein, 25 aa, Artificial Sequence
  source: /note="Description of Artificial Sequence: Synthetic peptide"
  SITE (1)..(20): /note="This region may encompass 0-4 'Gly Gly Gly Gly Gln' repeating units"
GGGGQGGGGQGGGGQGGGGQGGGGS

SEQ ID NO: 32 - protein, 25 aa, Artificial Sequence
  source: /note="Description of Artificial Sequence: Synthetic peptide"
  SITE (1)..(20): /note="This region may encompass 0-4 'Gly Gly Gly Gly Ala' repeating units"
GGGGAGGGGAGGGGAGGGGAGGGGS

SEQ ID NO: 33 - protein, 25 aa, Artificial Sequence
  source: /note="Description of Artificial Sequence: Synthetic peptide"
  SITE (6)..(25): /note="This region may encompass 0-4 'Pro Gly Gly Gly Ser' repeating units"
GGGGSPGGGSPGGGSPGGGSPGGGS

SEQ ID NO: 34 - protein, 15 aa, Artificial Sequence
  source: /note="Description of Artificial Sequence: Synthetic peptide"
GGGGAGGGGAGGGGS

SEQ ID NO: 35 - protein, 15 aa, Artificial Sequence
  source: /note="Description of Artificial Sequence: Synthetic peptide"
GGGGQGGGGQGGGGS

SEQ ID NO: 36 - protein, 15 aa, Artificial Sequence
  source: /note="Description of Artificial Sequence: Synthetic peptide"
GGGPSGGGPSGGGGS

SEQ ID NO: 37 - protein, 15 aa, Artificial Sequence
  source: /note="Description of Artificial Sequence: Synthetic peptide"
GGGGSPGGGSPGGGS

SEQ ID NO: 38 - protein, 8 aa, Homo sapiens
FSDLWKLL

SEQ ID NO: 39 - protein, 12 aa, Artificial Sequence
  source: /note="Description of Artificial Sequence: Synthetic peptide"
TSFAEYWNLLSP

SEQ ID NO: 40 - protein, 16 aa, Artificial Sequence
  source: /note="Description of Artificial Sequence: Synthetic peptide"
  MOD_RES (9)..(9): (R)-2-(7'-octenyl)alanine
  MOD_RES (16)..(16): (S)-2-(4'-pentenyl)alanine
LSQETFSDXWKLLPEX

SEQ ID NO: 41 - protein, 16 aa, Artificial Sequence
  source: /note="Description of Artificial Sequence: Synthetic peptide"
  MOD_RES (5)..(5): (R)-2-(7'-octenyl)alanine
  MOD_RES (12)..(12): (S)-2-(4'-pentenyl)alanine
LSQEXFSDLWKXLPEN

SEQ ID NO: 42 - protein, 16 aa, Artificial Sequence
  source: /note="Description of Artificial Sequence: Synthetic peptide"
  MOD_RES (4)..(4): (R)-2-(7'-octenyl)alanine
  MOD_RES (11)..(11): (S)-2-(4'-pentenyl)alanine
LSQXTFSDLWXLLPEN

SEQ ID NO: 43 - protein, 16 aa, Artificial Sequence
  source: /note="Description of Artificial Sequence: Synthetic peptide"
  MOD_RES (7)..(7): (R)-2-(7'-octenyl)alanine
  MOD_RES (14)..(14): (S)-2-(4'-pentenyl)alanine
LSQETFXDLWKLLXEN

SEQ ID NO: 44 - protein, 16 aa, Artificial Sequence
  source: /note="Description of Artificial Sequence: Synthetic peptide"
  MOD_RES (7)..(7): (R)-2-(7'-octenyl)alanine
  MOD_RES (14)..(14): (S)-2-(4'-pentenyl)alanine
LSQETFXNLWKLLXQN

SEQ ID NO: 45 - protein, 16 aa, Artificial Sequence
  source: /note="Description of Artificial Sequence: Synthetic peptide"
  MOD_RES (7)..(7): (R)-2-(7'-octenyl)alanine
  MOD_RES (14)..(14): (S)-2-(4'-pentenyl)alanine
LSQQTFXNLWRLLXQN

SEQ ID NO: 46 - protein, 16 aa, Artificial Sequence
  source: /note="Description of Artificial Sequence: Synthetic peptide"
  MOD_RES (7)..(7): (R)-2-(7'-octenyl)alanine
  MOD_RES (14)..(14): (S)-2-(4'-pentenyl)alanine
QSQQTFXNLWKLLXQN

SEQ ID NO: 47 - protein, 16 aa, Artificial Sequence
  source: /note="Description of Artificial Sequence: Synthetic peptide"
  MOD_RES (7)..(7): (R)-2-(7'-octenyl)alanine
  MOD_RES (14)..(14): (S)-2-(4'-pentenyl)alanine
QSQQTFXNLWRLLXQN

SEQ ID NO: 48 - protein, 14 aa, Artificial Sequence
  source: /note="Description of Artificial Sequence: Synthetic peptide"
  MOD_RES (1)..(1): Any amino acid
  MOD_RES (4)..(4): (R)-2-(7'-octenyl)alanine
  MOD_RES (5)..(5): Any amino acid
  VARIANT (7)..(7): /replace="F" or "3-(2-naphthyl)-L-alanine"
  VARIANT (10)..(10): /replace="Cyclobutylalanine"
  MOD_RES (11)..(11): (S)-2-(4'-pentenyl)alanine
  MOD_RES (12)..(12): Any amino acid
  SITE (1)..(14): /note="Variant residues given in the sequence have no preference with respect to those in the annotations for variant positions"
  source: /note="See specification as filed for detailed description of substitutions and preferred embodiments"
XTFXXYWAQLXXAA

SEQ ID NO: 49 - protein, 14 aa, Artificial Sequence
  source: /note="Description of Artificial Sequence: Synthetic peptide"
  MOD_RES (4)..(4): (R)-2-(4'-pentenyl)alanine
  MOD_RES (11)..(11): (S)-2-(7'-octenyl)alanine
LTFXEYWAQLXSAA

SEQ ID NO: 50 - protein, 14 aa, Artificial Sequence
  source: /note="Description of Artificial Sequence: Synthetic peptide"
  MOD_RES (4)..(4): (R)-2-(4'-pentenyl)alanine
  MOD_RES (10)..(10): Cyclobutylalanine
  MOD_RES (11)..(11): (S)-2-(7'-octenyl)alanine
LTFXEYWAQXXSAA

SEQ ID NO: 51 - protein, 17 aa, Artificial Sequence
  source: /note="Description of Artificial Sequence: Synthetic peptide"
  MOD_RES (3)..(3): (S)-2-(4'-pentenyl)alanine
  MOD_RES (7)..(7): (S)-2-(4'-pentenyl)alanine
  MOD_RES (11)..(11): (S)-2-(4'-pentenyl)alanine
  MOD_RES (15)..(15): (S)-2-(4'-pentenyl)alanine
  source: /note="See specification as filed for detailed description of substitutions and preferred embodiments"
ATXNVLXWLYXAVIXGD

SEQ ID NO: 52 - protein, 19 aa, Artificial Sequence
  source: /note="Description of Artificial Sequence: Synthetic peptide"
  MOD_RES (7)..(7): Norleucine
  MOD_RES (8)..(8): (S)-2-(4'-pentenyl)alanine
  MOD_RES (12)..(12): (S)-2-(4'-pentenyl)alanine
  source: /note="See specification as filed for detailed description of substitutions and preferred embodiments"
ANLNAGXXLGSXAATVELQ

SEQ ID NO: 53 - protein, 6 aa, Artificial Sequence
  source: /note="Description of Artificial Sequence: Synthetic 6xHis tag"
HHHHHH

SEQ ID NO: 54 - protein, 35 aa, Artificial Sequence
  source: /note="Description of Artificial Sequence: Synthetic polypeptide"
  MOD_RES (7)..(7): Norleucine
  MOD_RES (8)..(8): (S)-2-(4'-pentenyl)alanine
  MOD_RES (12)..(12): (S)-2-(4'-pentenyl)alanine
  MOD_RES (20)..(20): Beta-alanine
  MOD_RES (25)..(25): (R)-2-(7'-octenyl)alanine
  MOD_RES (32)..(32): (S)-2-(4'-pentenyl)alanine
ANLNAGXXLGSXAATVELQXALTFXEYWAQLXSAA

SEQ ID NO: 55 - protein, 34 aa, Artificial Sequence
  source: /note="Description of Artificial Sequence: Synthetic polypeptide"
  MOD_RES (4)..(4): (R)-2-(7'-octenyl)alanine
  MOD_RES (11)..(11): (S)-2-(4'-pentenyl)alanine
  MOD_RES (15)..(15): Beta-alanine
  MOD_RES (22)..(22): Norleucine
  MOD_RES (23)..(23): (S)-2-(4'-pentenyl)alanine
  MOD_RES (27)..(27): (S)-2-(4'-pentenyl)alanine
LTFXEYWAQLXSAAXANLNAGXXLGSXAATVELQ

(19) United States
(12) Patent Application Publication
(10) Pub. No.: US 2023/0330238 A1
(43) Pub. Date: Oct. 19, 2023

(54) CHIMERIC CONJUGATES FOR DEGRADATION OF VIRAL AND HOST PROTEINS AND METHODS OF USE

(71) Applicant: Dana-Farber Cancer Institute, Inc., Boston, MA (US)

(72) Inventors: Henry D. Herce, Brookline, MA (US); Loren D. Walensky, Newton, MA (US)

(21) Appl. No.: 18/030,433

(22) PCT Filed: Oct. 14, 2021
(86) PCT No.: PCT/US2021/054954

§ 371 Date: Apr. 5, 2023

Related U.S. Application Data
(60) Provisional application No. 63/091,769, filed on Oct. 14, 2020.

(51) Int. Cl. A61K 47/55; A61K 31/454; A61K 31/427; A61K 38/04; A61K 31/4166; A61P 31/14

(52) U.S. Cl. CPC A61K 47/55; A61K 31/4166; A61K 31/427; A61K 31/454; A61K 38/04; A61P 31/14

(57) ABSTRACT

The present application describes chimeras which target and degrade essential viral proteins or host proteins involved in viral pathogenesis. In particular, the chimeras of this application combine a moiety that binds to a target protein (such as a coronaviral papain-like protease (PLpro), main protease (Mpro), or other non-structural proteins (e.g., NSP9 or NSP12); or a host protein, such as bromodomain 2, bromodomain 3, or bromodomain 4)), with a moiety that recruits a protein degrader, thereby degrading the target protein. In some instances, the chimera simultaneously induces p53, which itself has anti-viral activity, by engaging HDM2 as the protein degrader. The disclosure also relates to methods of using such chimeras in the prevention and treatment of viral infections, particularly viral infections (such as COVID-19) caused by coronaviruses (such as SARS-CoV-2).

### CROSS-REFERENCE TO RELATED APPLICATION

This application claims the benefit of priority of U.S. Provisional Appl. No. 63/091,769, filed Oct. 14, 2020, the content of which is incorporated herein by reference in its entirety.

### SEQUENCE LISTING

The present specification is being filed with a computer readable form (CRF) copy of the Sequence Listing. The CRF entitled 00530-0409WO1_SL.txt, which was created on Oct. 14, 2021 and is 25,132 bytes in size, is incorporated herein by reference in its entirety.

### TECHNICAL FIELD

This disclosure relates to chimeric conjugates, i.e., proteolysis targeting chimeras (PROTACs) that target the degradation of viral proteins, for example essential viral proteins such as coronaviral papain-like protease (PLpro), main protease (Mpro), other non-structural proteins (e.g., NSP9 or NSP12); or a host protein involved in viral pathogenesis, such as a bromodomain and extraterminal domain (BET) protein (e.g., bromodomain 2, bromodomain 3, or bromodomain 4). The disclosure also relates to methods of using such chimeric conjugates in the treatment and prevention of viral infections, particularly viral infections caused by coronaviruses. Also disclosed are peptides that inhibit viral proteins such as coronaviral papain-like protease (PLpro), main protease (Mpro) as well as other non-structural viral proteins (e.g., NSP9 or NSP12) that to inhibit viral maturation and replication. These peptides can be used to treat viral infections (e.g., coronaviral infections).

### BACKGROUND

PLpro and Mpro are essential enzymes in the life cycle of RNA viruses, including coronaviruses such as SARS-CoV-2. PLpro is a multifunctional cysteine protease that processes the viral polyprotein and host cell proteins by hydrolyzing the peptide and isopeptide bonds in viral and cellular substrates, required for viral replication (Baez-Santos, Y.M., et al. J Virol 88, 12511-27 (2014)). Mpro is the main proteolytic processing enzyme of SARS-CoV-2 which plays a major role in the autoprocessing proteolytic reactions that yield mature proteins (NSP5-16) essential to the viral life cycle (Chang, G.G. Molecular Biology of the SARS-Coronavirus, 115-128 (2009). Coronaviral nonstructural proteins such as NSP9 and NSP12 are essential proteins involved in viral replication. Several compounds have been developed to inhibit the protease activity of PLpro and Mpro and inhibit other viral NSPs (see e.g., Baez-Santos et al. 2014, Jin Z Nature 2020; Anand, K., et al. Science 300, 1763-7 (2003); Ghosh, A.K. et al., J Med Chem 52, 5228-40 (2009); Baez-Santos, Y.M., et al. Antiviral Res 115, 21-38 (2015)). However, these inhibitors are not sufficiently potent and thus only partial inhibition is achieved. There is an urgent need to develop efficient therapeutics that target viruses such as coronaviruses, e.g., SARS-CoV-2, and enable the host system to fight off infections such as COVID-19.

### SUMMARY

This disclosure relates to the characterization and use of chimeric conjugates (“chimeras”), i.e., proteolysis targeting chimeras (PROTACs) that target the degradation of viral proteins or host proteins involved in viral pathogenesis. In some instances, these viral proteins are essential proteins for viral replication, infectivity, or pathogenesis. Such chimeras can be used to degrade an essential viral protein of any RNA virus, thereby treating an infection caused by such RNA virus. In particular, the chimeras of this disclosure can be used to degrade essential coronaviral proteins, such as, papain-like protease (PLpro), main protease (Mpro), or another non-structural protein (e.g., NSP9 and NSP12). The chimeras of this disclosure can also target the degradation of host proteins involved in viral pathogenesis, in particular, bromodomain 2, bromodomain 3 and bromodomain 4 (BRD2, BRD3, and BRD4, respectively). For example, this disclosure encompasses each of the molecules shown in FIGS. 7 and 14A.

This disclosure also describes methods of using such chimeras in the treatment of viral infections, particularly viral infections caused by coronaviruses (e.g., COVID-19). The chimeras of this disclosure combine a viral or host protein targeting moiety (e.g, a peptide, a stapled peptide, a small molecule, a small molecule derivatized with a warhead, or a nucleotide analog) and a protein degradation-inducing moiety (e.g., a peptide, a stapled peptide, or a small molecule that binds to or recruits the protein degrader). Thus, such chimeras can be bifunctional stapled peptide-small molecule conjugates, peptide-small molecule conjugates, stapled peptide-peptide conjugates, peptide-stapled peptide conjugates stapled peptide-stapled peptide conjugates, peptide-peptide conjugates, small molecule-stapled peptide conjugates, and small molecule-small molecule conjugates, etc. that can be used to target any viral protein of interest (e.g., coronaviral PLpro, Mpro, NSP9, NSP12, etc.) or any host protein involved in viral pathogenesis (e.g., BRD2, BRD3, BRD4, etc).

This disclosure provides compositions and methods for not only degrading a viral protein of interest (e.g., coronaviral PLpro, Mpro, NSP9, NSP12, etc.) or any host protein involved in viral pathogenesis (e.g., BRD2, BRD3, BRD4, etc), but also to increase the amount of p53 protein that is not complexed with HDM2 and/or HDMX. This allows for the p53-mediated suppression of viral replication and/or pathogenesis. Thus, the present compositions provide for an additive or synergistic increase in anti-virus (e.g., anti-SARS) activity.

Also disclosed are peptides that can bind and inhibit viral proteins such as M pro and NSP9.

In a first aspect, this disclosure features a chimera, comprising a first moiety attached to a second moiety, wherein the first moiety and second moiety are directly attached to each other or attached to each other via a linker. The first moiety binds to a first protein targeted for degradation. In some instances, the first protein is selected from a coronaviral protease, a coronaviral non-structural protein (NSP), or a bromodomain and extraterminal domain (BET) protein. The second moiety binds to a second protein, wherein the second protein is, or recruits, “a protein degrader.” In some instances, the second protein is an E3 ubiquitin ligase.

In another aspect, the disclosure relates to a chimera, comprising a means for binding a viral protein (Mpro, PLpro, NSP9, NSP12) or a host protein (e.g., BRD2, BRD3, or BRD4) attached to a second moiety directly or via a linker. The second moiety binds to a second protein, wherein the second protein is, or recruits, “a protein degrader.” In some instances, the second protein is an E3 ubiquitin ligase.

In another aspect the disclosure provides a first moiety which binds to a first protein targeted for degradation (e.g., a coronaviral protease, a coronaviral non-structural protein (NSP), or a bromodomain and extraterminal domain (BET) protein) attached directly or via a linker to a means for binding an E3 ligase (e.g., HDM2, VHL, cereblon, XIAP, cIAP, COP1).

In some instances, the first moiety and the second moiety are attached to each other via a linker. For example, the linker can be a peptide linker, a chemical linker, a Glycine-Serine linker such as (G4S)₃ (SEQ ID NO: 26) or (G4S)₅ (SEQ ID NO: 27), a beta-alanine (Z) linker, a beta-alanine and alanine (ZA) linker, or a polyethylene glycol linker.

In certain instances, the first moiety comprises a small molecule, a small molecule derivatized with a warhead, a peptide, a stapled peptide, a peptide derivatized with a warhead, a stapled peptide derivatized with a warhead, or a nucleotide analog.

In some instances, the coronaviral protease is papain-like protease (PLpro), or main protease (Mpro); the coronaviral NSP is NSP9 or NSP12; and the BET protein is bromodomain 2 (BRD2), bromodomain 3 (BRD3), or bromodomain 4 (BRD4).

In one instance, the coronaviral protease is PLpro and the first moiety binds to PLpro. The first moiety that binds to PLpro can be a PLpro inhibitor. In some cases, the PLpro inhibitor is GRL-0617 or a PLpro-binding analog thereof, disulfiram, or a PLpro-binding thiopurine analog.

In certain instances, the coronaviral protease is Mpro and the first moiety binds to Mpro. In some cases, the Mpro inhibitor is Lopinavir, Ritonavir, Darunavir, ASC09, ASC09F, GC376, GC813, Ebselen carboxylic acid, or a peptide. In certain cases, the peptide comprises an amino acid sequence with at least 30%, 40%, 50%, 60%, 70%, 80%, 90%, 95%, or 100% identity to the sequence set forth in SEQ ID NO: 2, or SEQ ID NO: 3, wherein the peptide binds and inhibits Mpro. In certain cases, the peptide comprises an amino acid sequence set forth in SEQ ID NO: 2, or SEQ ID NO: 3 except for 1 to 5 amino acid substitutions, wherein the peptide binds and inhibits Mpro. The substitutions can be with a conservative amino acid. In some instances, the substitutions can be with a nonconservative amino acid so long as the peptide binds and inhibits Mpro.

In some instances, the BET protein is BRD4 and the first moiety binds to the BET protein. In some cases, the first moiety that binds to the BET protein is a BET protein inhibitor. In certain cases, the BET protein inhibitor is JQ1, ABBV-075, I-BET151, I-BET726, OTX015, or PFI-1, or analogs thereof that bind BRD4, BRD3 and/or BRD2.

In some instances, the coronaviral NSP is NSP9 and the first moiety binds to NSP9. In some cases, the first moiety that binds to NSP9 is an NSP9 inhibitor. In certain cases, the first moiety is a peptide comprising an amino acid sequence with at least 30%, 40%, 50%, 60%, 70%, 80%, 90%, 95%, or 100% identity to the sequence set forth in SEQ ID NO: 4 or SEQ ID NO: 5, wherein the peptide binds NSP9. In certain cases, the peptide comprises an amino acid sequence set forth in SEQ ID NO: 4, or SEQ ID NO: 5 except for 1 to 5 amino acid substitutions, wherein the peptide binds NSP9. The substitutions can be with a conservative amino acid. In some instances, the substitutions can be with a nonconservative amino acid so long as the peptide binds NSP9.

In certain instances, the coronaviral NSP is NSP12 and the first moiety binds to NSP12. In some cases, the first moiety that binds to NSP12 is an NSP12 inhibitor. In certain cases, the first moiety is remdesivir acid or an analog thereof that binds NSP12, or sofosbuvir acid or an analog thereof that binds NSP12.

In some instances, the second protein is human double minute 2 (HDM2), Von Hippel-Lindau (VHL), Cereblon, X-linked inhibitor of apoptosis protein (XIAP), cellular inhibitor of apoptosis protein (cIAP), or Constitutive photomorphogenic 1 (COP1). In some cases, the second moiety comprises a peptide, a stapled peptide, or a small molecule that binds to or recruits the protein degrader. In some cases, the second moiety comprises a cereblon binding moiety that is a small molecule. In some cases, the small molecule is selected from a group consisting of thalidomide, pomalidomide, lenalidomide, avadomide, and analogs thereof that bind cereblon. In some cases, the second moiety comprises a thalidomide moiety. In one case, the thalidomide moiety comprises the structure provided below:

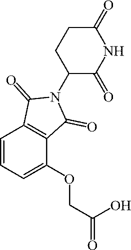 [SMILES: O=C(O)COc1cccc2c1C(=O)N(C1CCC(=O)NC1=O)C2=O]

or a cereblon-binding analog thereof. In some cases, the thalidomide moiety comprises the structure provided below:

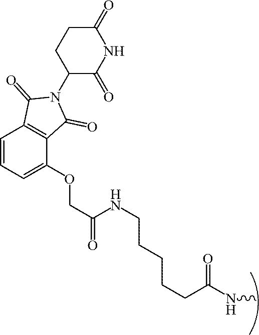

or a cereblon-binding analog thereof. In certain cases, the thalidomide moiety comprises the structure provided below:

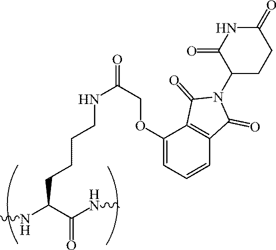

or a cereblon-binding analog thereof.

In some instances, the second moiety comprises a VHL binding moiety, optionally selected from a group consisting of VH 032 and VHL-binding analogs thereof. In certain cases, the VHL binding moiety comprises the structure below:

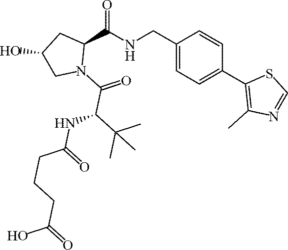 [SMILES: Cc1ncsc1-c1ccc(CNC(=O)[C@@H]2C[C@@H](O)CN2C(=O)[C@@H](NC(=O)CCCC(=O)O)C(C)(C)C)cc1]

or a VHL-binding analog thereof. In some cases, the VHL binding moiety comprises the structure below:

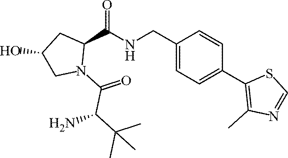 [SMILES: Cc1ncsc1-c1ccc(CNC(=O)[C@@H]2C[C@@H](O)CN2C(=O)[C@@H](N)C(C)(C)C)cc1]

or a VHL-binding analog thereof.

In some instances, the second moiety comprises an HDM2 binding moiety. In certain cases, the HDM2 binding moiety comprises a peptide or a stapled peptide or an otherwise chemically-stabilized peptide of the transactivation domain of p53 that binds HDM2 and/or HDMX. In some cases, the HDM2 binding moiety is a stapled peptide that is ATSP-7041, SP645, or an HDM2-binding variant thereof.

In certain cases, the stapled peptide comprises the sequence LTF(R8)EYWAQ#(S5)SAA (SEQ ID NO: 7) wherein (R8) is (R)-2-(7′-octenyl)alanine, # is cyclobutylalanine, and (S5) is (S)-2-(4′-pentenyl)alanine, or an HDM2-binding variant thereof. In certain cases, the stapled peptide comprises an amino acid sequence with at least 30%, 40%, 50%, 60%, 70%, 80%, 90%, 95%, or 100% identity to the sequence set forth in SEQ ID NO: 7, wherein the stapled peptide binds HDM2. In some cases, the stapled peptide comprises an amino acid sequence with at least 30%, 40%, 50%, 60%, 70%, 80%, 90%, or 95%, identity to the sequence set forth in SEQ ID NO: 7, wherein the amino acids on the interacting face of the peptide are not substituted, and wherein the stapled peptide binds HDM2. In some cases, the stapled peptide comprises an amino acid sequence with at least at least 30%, 40%, 50%, 60%, 70%, 80%, 90%, or 95%, identity to the sequence set forth in SEQ ID NO: 7, wherein one or more of the amino acids on the interacting face of the peptide are substituted with a conservative amino acid and wherein the stapled peptide binds HDM2. In certain cases, the stapled peptide comprises an amino acid sequence set forth in SEQ ID NO: 7, except for 1, 2, 3, 4, 5, or 6 amino acid substitutions, wherein the stapled peptide binds HDM2. The substitutions can be with a conservative amino acid(s). In some instances, the substitutions can be with a nonconservative amino acid (s) so long as the peptide binds HDM2.

In certain cases, the stapled peptide comprises the sequence LTF(R8)EYWAQL(S5)SAA (SEQ ID NO: 1) wherein (R8) is (R)-2-(7′-octenyl)alanine, # is cyclobutylalanine, and (S5) is (S)-2-(4′-pentenyl)alanine, or an HDM2-binding variant thereof. In certain cases, the stapled peptide comprises an amino acid sequence with at least 30%, 40%, 50%, 60%, 70%, 80%, 90%, 95%, or 100% identity to the sequence set forth in SEQ ID NO: 1, wherein the stapled peptide binds HDM2. In some cases, the stapled peptide comprises an amino acid sequence with at least 30%, 40%, 50%, 60%, 70%, 80%, 90%, or 95%, identity to the sequence set forth in SEQ ID NO: 1, wherein the amino acids on the interacting face of the peptide are not substituted, and wherein the stapled peptide binds HDM2. In some cases, the stapled peptide comprises an amino acid sequence with at least at least 30%, 40%, 50%, 60%, 70%, 80%, 90%, or 95%, identity to the sequence set forth in SEQ ID NO: 1, wherein one or more of the amino acids on the interacting face of the peptide are substituted with a conservative amino acid and wherein the stapled peptide binds HDM2. In certain cases, the stapled peptide comprises an amino acid sequence set forth in SEQ ID NO: 1, except for 1, 2, 3, 4, 5, or 6 amino acid substitutions, wherein the stapled peptide binds HDM2. The substitutions can be with a conservative amino acid(s). In some instances, the substitutions can be with a nonconservative amino acid (s) so long as the peptide binds HDM2.

In some cases, the HDM2 binding moiety is Nutlin-3a or an HDM2-binding analog thereof. In some cases, the HDM2 binding moiety comprises the structure below:

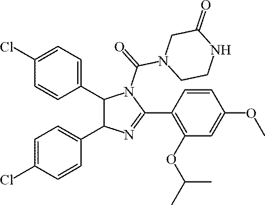 [SMILES: COc1ccc(C2=NC(c3ccc(Cl)cc3)C(c3ccc(Cl)cc3)N2C(=O)N2CCNC(=O)C2)c(OC(C)C)c1]

In certain cases, the second moiety comprises an XIAP binding moiety that is A410099.1 or an XIAP-binding analog thereof. In some cases, the XIAP binding moiety comprises the structure below:

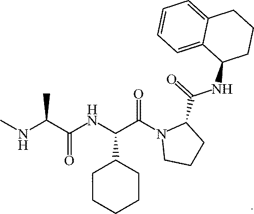

In certain cases, the second moiety comprises cIAP binding moiety that is SM-1295, SM-1280 or a cIAP-binding analog thereof.

In some cases, the second moiety comprises a peptide that binds a WD40-repeat protein that is a substrate adaptor for an E3 ubiquitin ligase. The peptide comprises a modified version of a natural binding sequence or a natural binding consensus sequence of an amino acid sequence that binds to the WD40-repeat protein. The modified version comprises at least one amino acid substitution, at least one amino acid deletion, at least one amino acid insertion, or any combination thereof within the natural binding consensus sequence. In certain cases, the WD40-repeat protein is a substrate adaptor for the E3 ubiquitin ligase HDM2 or VHL. In some cases, the natural binding consensus sequence SEQ ID NOs.: 14 or 15, or a variant thereof, wherein the variant differs from the consensus sequence at one to six amino acid positions.

In certain instances, the second moiety comprises a COP1 binding moiety. In some cases, the COP1 binding moiety is a peptide that is a Tribbles Pseudokinase 1 (Trib1) peptide or a COP1-binding variant thereof. In some cases, the peptide comprises the sequence DQIVPEY (SEQ ID NO: 6), or a peptide comprising an amino acid sequence with at least 30% identity to the sequence set forth in SEQ ID NO: 6.

In some instances, the protein degrader facilitates the degradation of the first protein. For example, an E3 ubiquitin ligase facilitates, the ubiquitylation and degradation of the first protein.

In another aspect, the disclosure relates to a chimera comprising a molecule having the structure of any one of the molecules depicted in FIG. 7 or FIG. 14A.

In another aspect, this disclosure features a pharmaceutical composition comprising a peptide or a chimera disclosed herein and a pharmaceutically acceptable carrier. In some cases, the pharmaceutical composition is formulated for oral, intravenous, topical, buccal, rectal, parenteral, intraperitoneal, intradermal, subcutaneous, intramuscular, transdermal, intranasal, pulmonary, or intratracheal administration.

In yet another aspect, this disclosure provides a method of treating or preventing a viral infection caused by a coronavirus in a subject in need thereof. The method comprises administering to the subject a therapeutically effective amount of a peptide, a chimera, or a pharmaceutical composition disclosed herein.

In another aspect, the disclosure features a method for both blocking viral replication and reducing viral infectivity of coronavirus in a subject in need thereof. The method comprises administering to the subject a therapeutically effective amount of a peptide, a chimera, or a pharmaceutical composition disclosed herein. In some cases, the coronavirus is Middle East Respiratory Syndrome coronavirus (MERS-CoV), severe acute respiratory syndrome coronavirus (SARS-CoV), or SARS-CoV-2.

In another aspect, the disclosure provides a method for blocking the replication of SARS-CoV or SARS-CoV-2 in a subject in need thereof. The method comprises administering to the subject a therapeutically effective amount of a peptide, a chimera, or a pharmaceutical composition disclosed herein.

In another aspect, the disclosure features a method for treating or preventing an RNA virus infection in a subject in need thereof. The method comprising administering to the subject a therapeutically effective amount of a peptide, a chimera, or a pharmaceutical composition disclosed herein.

In some cases, the above methods can further comprise administering to the subject one or more agents selected from the group consisting of a corticosteroid, hydrocortisone, methylprednisolone, dexamethasone, remdesivir, an IL-6 inhibitor, an IL-1 inhibitor, a kinase inhibitor, a complement inhibitor, ivermectin, hydroxychloroquine, favipiravir, interferon-beta, and icatibant.

In some cases, the subject is selected from a group consisting of a human, a primate, a bat, a bird, a mouse, a turkey, a cow, a pig, a cat and a dog. In one instance, the subject is a human.

In another aspect, the disclosure provides a peptide comprising the amino acid sequence set forth in SEQ ID NOs.: 2 or 3, or a variant thereof (e.g., a peptide that differs from SEQ ID NO: 2 or 3 by 1, 2, 3, 4, 5, 6, or 7 amino acid substitutions or deletions), wherein the peptide binds and inhibits Mpro.

In another aspect, the disclosure features a stabilized (e.g., stapled, stitched) peptide comprising a peptide comprising the amino acid sequence set forth in SEQ ID NOs.: 2 or 3 with 1, 2, 3, 4, 5, or 6 amino acid substitutions, wherein at least two amino acid substitutions replace amino acids separated by three or six amino acids with non-natural amino acids, and wherein the peptide binds and inhibits Mpro. When the amino acid substitutions replace amino acids separated by three amino acids with non-natural amino acids, the non-natural amino acids are both S5. When the amino acid substitutions replace amino acids separated by six amino acids with non-natural amino acids, the non-natural amino acids are R8 [(R)-2-(7′-octenyl)alanine] and S5 [(S)-2-(4′-pentenyl)alanine], or R5 [(R)-2-(4′-pentenyl)alanine] and S8 [(S)-2-(7′-octenyl)alanine], respectively.

In some cases, the peptide or stabilized peptide is less than 50, 40, 35, 30, 25, 24, 23, or 21 amino acids in length.

In another aspect, the disclosure features a peptide comprising the amino acid sequence set forth in SEQ ID NOs.: 4 or 5, or a variant thereof, wherein the peptide binds NSP9. In some instances, the peptide inhibits dimerization of NSP9.

In another aspect, the disclosure provides a stabilized (e.g., stapled, stitched) peptide comprising a peptide comprising the amino acid sequence set forth in SEQ ID NOs.: 4 or 5 with 1, 2, 3, 4, 5, or 6 amino acid substitutions, wherein at least two amino acid substitutions replace amino acids separated by three or six amino acids with non-natural amino acids, and wherein the peptide binds NSP9. In some instances, the stabilized peptide inhibits dimerization of NSP9.

In some cases, the peptide or stabilized peptide is less than 50, 40, 35, 30, 25, 24, 23, 22, or 21 amino acids in length.

In another aspect, the disclosure features a pharmaceutical composition comprising peptide or stabilized peptide disclosed herein, and a pharmaceutically acceptable carrier.

In yet another aspect, this disclosure provides a method of treating or preventing a coronaviral infection in a subject in need thereof. The method involves administering to the subject a therapeutically effective amount of a peptide, a stabilized peptide, or a pharmaceutical composition described herein. In some cases, the subject is a human subject. In some cases, the subject is a cat, dog, horse, sheep, chicken, or cow.

The disclosure also features a pharmaceutical composition comprising a means for inhibiting Mpro or NSP9 and a pharmaceutically acceptable carrier.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

For the avoidance of any doubt it is emphasized that the expressions “in some embodiments”, “in a certain embodiments”, “in certain instances”, “in some instances”, “in a further embodiment”, “in one embodiment” and “in a further embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples are intended to be generalized to equivalents having the same function as those exemplified therein.

Other features and advantages of the invention will be apparent from the following detailed description and from the claims.

### BRIEF DESCRIPTION OF THE DRAWINGS

FIG. 1 is a schematic diagram showing the mechanism by which a PLpro and HDM2-directed stapled peptide-proteolysis-targeting chimeras (SP-PROTACs) degrades PLpro protein (an essential protease of SARS-CoV-2) and increases p53 levels in host cells. The HDM2-binding stapled peptide displaces p53 from an HDM2/HDMX-p53 complex thereby increasing the levels of free p53 (denoted by the upward arrow) while the PLpro binding portion of the SP-PROTAC binds PLpro and brings it in proximity to the E3 ubiquitin ligase, HDM2, which results in the ubiquitination and ultimate degradation of PLpro (denoted by the downward directed arrow). Ub: Ubiquitin; Hdm2: human double minute 2; p53: Tumor protein p53; PLpro: Papain-Like Protease; E2: Ubiquitin-conjugating enzyme.

FIG. 2 top panel shows the chemical structures of exemplary unnatural amino acids used to generate various kinds of stapled peptides.

FIG. 2 middle panel shows the generation of stapled peptides with staples of various lengths spanning discrete distances along the length of a peptide helix (i, i+3, i, i+4, and i, i+7)

FIG. 2 bottom panel illustrates how a staple walk is achieved along a peptide sequence.

FIG. 3 shows various kinds of double and triple stapling strategies along with exemplary staple walks for generating stapled peptides.

FIG. 4 illustrates exemplary staple walks for generating stitched peptides.

FIG. 5 shows the various ways in which the peptide ligand component(s) of SP-PROTACs can be optimized by mutagenesis, differential staple or stitch insertion(s), and mutations of the peptide sequence by substation, deletion, addition, or derivatization.

FIG. 6 shows an exemplary ring closing metathesis (RCM) stapling reaction that can be employed to generate stapled peptides (in this case an i, i+7 stapled peptide).

FIG. 7 depicts the structure of two stapled peptide chimeras designed to target an essential SARS-CoV-2 protein (PLpro) and a host protein (BRD4 and/or BRD2) that binds to a SARS-CoV-2 protein: BRD4 (JQ1/SP645 SP-PROTAC, top) and PLpro (GRL0617/SP645 SP-PROTAC, bottom). The stapled peptide (SP645 (LTF(R8)EYWAQL(S5)SAA (SEQ ID NO: 1)) is incorporated to bind and recruit HDM2, an E3 ligase, and the small molecule (JQ1 or GRL0617) is included to bind to a target protein (BRD4 or PLpro, respectively). A linker of variable length and composition is installed between the two binding components of the chimera to ensure optimal engagement of the protein targets.

FIG. 8A shows the levels of α-helical stabilization of various HDM2-binding stapled p53 peptides (SAH-p53-1 to 4; SEQ ID NOs.: 40-43 respectively) compared to wild type p53₁₄₋₂₉ (SEQ ID NO: 11).

FIG. 8B depicts the affinity of various stapled p53 peptides (SAH-p53-1 to 4; SEQ ID NOs.: 40-43) for HDM2 compared to wild type p53₁₄₋₂₉ (SEQ ID NO: 11).

FIG. 9A represents a decay plot versus time for the anti-HIV therapeutic Enfuvirtide, singly-stapled Enfuvirtide, and doubly stapled Enfuvirtide, highlighting the capacity of stapling to confer protease resistance to peptides.

FIG. 9B shows traces to determine complex formation as assessed by size exclusion chromatography (SEC) for various molecules and chimeras. BRD4-directed SP-PROTAC + HDM2 (fifth trace) induced complex formation, whereas HDM2 alone (top trace); BRD4 alone (second trace); HDM2+BRD4 (third trace); HDM2+BRD4+SP645+JQ1 (fourth trace) did not.

FIG. 10A are images that show that unlike HeLa cells treated with vehicle (top row), cells treated with SP-PROTAC-BRD4 exhibit relocalization of HDM2 from the cytosol (diffuse staining) to the nuclear lamina where BRD4 is experimentally anchored (bottom row).

FIG. 10B is an image of a western blot of PLpro protein and ubiquitylated-PLpro protein levels in the presence of vehicle or SP-PROTAC-PLpro as determined by an in vitro ubiquitylation assay.

FIG. 11A is an image of a western blot of BRD4 and p53 levels in SJSA-1 cells exposed to various concentrations of SP-PROTAC-BRD4. Actin antibody is used as the loading control.

FIG. 11B is an image of a western blot of BRD4 and p53 levels in SJSA-1 cells exposed to various concentrations of SP-PROTAC-BRD4 in the presence of the selective proteasome inhibitor, carfilzomib. Actin antibody is used as the loading control.

FIG. 12A shows the levels of BRD proteins (BRD2/3/4) and the p53 transcriptional target, HDM2, in cells treated with SP645 alone.

FIG. 12B depicts the levels of BRD proteins (BRD2/3/4) and the p53 transcriptional target, HDM2, in cells treated with JQ1 alone.

FIG. 12C depicts the levels of BRD proteins (BRD2/3/4) and the p53 transcriptional target, HDM2, in cells treated with SP-PROTAC-BRD4.

FIG. 13A shows the percentage (%) viability of SJSA-1 cells in the presence of SP645, JQ1, or SP-PROTAC-BRD4. Data represents mean ± SEM., *p<0.05 pairwise for each group.

FIG. 13B depicts percentage (%) of Vero E6 cells that were infected with SARS-CoV-2 in the presence of various concentrations of SP-PROTAC-PLpro1. Data represents mean ± SEM., *p<0.05 first three bars vs. last two bars.

FIG. 14A depicts the compositions of two SP-PROTACS, SP-PROTAC-NSP9-1 (SEQ ID NO: 54) and SP-PROTAC-NSP9-2 (SEQ ID NO: 55), designed to induce the targeted degradation of viral NSP9 by MDM2 of the infected host cell.

FIG. 14B demonstrates that SP-PROTAC-NSP9-1 and SP-PROTAC-NSP9-2 can recruit MDM2 to ubiquitinate the viral target protein USP9, as demonstrated by an in vitro ubiquitination assay.

### DETAILED DESCRIPTION

This disclosure is based on the finding that proteolysis-targeting chimeras (PROTACs), e.g., stapled-peptide proteolysis-targeting chimeras (SP-PROTACs), can be used to degrade viral proteins (e.g., essential viral proteins critical for viral pathogenesis). In some instances, these PROTACs simultaneously induce a p53 surge in host cells to impede viral replication and pathogenesis. The disclosure features molecules that bring an E3 ubiquitin ligase in proximity to an RNA virus (e.g., coronavirus) protein target to induce degradation (e.g., for therapeutic purposes). The viral target for degradation may be any viral protein (e.g., an essential viral protein such as a coronaviral protease or other non-structural protein (NSP)), or a host protein that facilitates viral pathogenesis (e.g., a bromodomain and extraterminal domain (BET) protein)). In some cases, the viral protein is coronaviral papain-like protease (PLpro), or main protease (Mpro); the coronaviral NSP is NSP9 or NSP12. In certain cases, the BET protein is bromodomain 2 (BRD2), bromodomain 3 (BRD3) or bromodomain 4 (BRD4). In other cases, the host protein is Sec61, an endoplasmic reticulum membrane protein translocon.

This disclosure provides chimeras that act as protein degradation inducing moieties, by combining a first moiety that targets a viral protein target (e.g., binds to a viral protein (e.g., an essential viral protein) or a host protein that facilitates viral pathogenesis), with a second moiety (e.g., a ligand) that binds to a protein that is, or recruits, “a protein degrader.” For example, the second moiety, recruits an enzyme or a complex that catalyzes the ubiquitination of the target, labeling in this way the target for degradation, which in turn is degraded by the proteasome. In some cases, the “protein degrader” is an E3 ligase that ubiquitylates and thus targets the viral protein for degradation.

The first moiety can include a small molecule, a small molecule derivatized with a warhead, a peptide, a stapled peptide, a peptide or stapled peptide derivatized with a warhead, or a nucleotide analog that binds to an essential viral protein or a host protein. In some cases, the first moiety is a PLpro binder, such as GRL-0617 or a PLpro-binding analog thereof, disulfiram, or a PLpro-binding thiopurine analog. In other cases, the first moiety is an Mpro binder, such as Lopinavir, Ritonavir, Darunavir, ASC09, GC376, GC813, Ebselen carboxylic acid, or a peptide comprising an amino acid sequence that is at least 30%, 40%, 50%, 60%, 70%, 80%, 85%, 90%, 95%, 96%, 97%, 98%, 99%, or 100% identical to the sequence set forth in SEQ ID NO: 2 or SEQ ID NO: 3. In some cases, the first moiety is an NSP9 binder, such as a peptide comprising an amino acid sequence that is at least 30%, 40%, 50%, 60%, 70%, 80%, 85%, 90%, 95%, 96%, 97%, 98%, 99%, or 100% identical to the sequence set forth in SEQ ID NO: 4 or SEQ ID NO: 5. In other cases, the first moiety is an NSP12 binder, such as remdesivir acid, sofosbuvir acid or analogs thereof. In certain cases, the first moiety is a BET protein binder such as JQ1, ABBV-075, I-BET151, I-BET726, OTX015, or PFI-1, or analogs thereof. In other cases, the first moiety is a Sec61 protein binder such as PS3061 or an analog thereof.

The second moiety can include a ligand (e.g., a peptide, a stapled peptide, or a small molecule) that binds to or recruits an E3 ubiquitin ligase, or a peptide that binds a WD40-repeat protein that is a substrate adaptor for an E3 ubiquitin ligase. An E3 ubiquitin ligase can be human double minute 2 (HDM2), Von Hippel-Lindau (VHL), Cereblon, X-linked inhibitor of apoptosis protein (XIAP), cellular inhibitor of apoptosis protein (cIAP), or Constitutive photomorphogenic 1 (COP1). In certain cases, an HDM2 binding moiety can comprise a peptide or a stapled peptide of the transactivation domain of p53 that binds HDM2 and/or HDMX, or a small molecule (e.g., Nutlin-3a or derivatives of Nutlin-3a such as RG7112 and RG7388 (Idasanutlin)) or an HDM2 binding analog thereof. In some cases, a Cereblon-binding moiety is thalidomide, pomalidomide, lenalidomide, avadomide, or a Cereblon-binding analog thereof. In other cases, a VHL-binding moiety is VH 032, or a VHL-binding analog thereof. In some cases, an XIAP-binding moiety can be A410099.1, or an XIAP-binding analog. In certain cases, a cIAP-binding moiety can be SM-1295 or SM-1280, or a cIAP-binding analog. In other cases, a COP1 binding moiety can comprise a peptide or stapled peptide 8 to 50 amino acid sequence from Tribbles Pseudokinase 1 (Trib1 -UniProtKB - Q96RU8). By combining peptide or small molecules that effectively bind and recruit a degrader protein with a small molecule, peptide, or nucleotide analog that targets an essential viral protein or host protein that aids in viral pathogenesis, this new class of degron chimeras provides novel compounds to combat viral infections. In some instances, the viral infection are coronaviral infections such as SARS-CoV-2. This disclosure also provides pharmaceutical compositions comprising the degron chimeras described. Further, this disclosure provides methods of treating viral infections, blocking viral replication, and reducing viral infectivity using such chimeras and compositions described. Because PLpro is also involved in viral mechanisms of evading the host immune system, a key advantage of the chimeras of this disclosure is that they are designed to both block viral replication and restore the capacity of the immune system to kill infected cells.

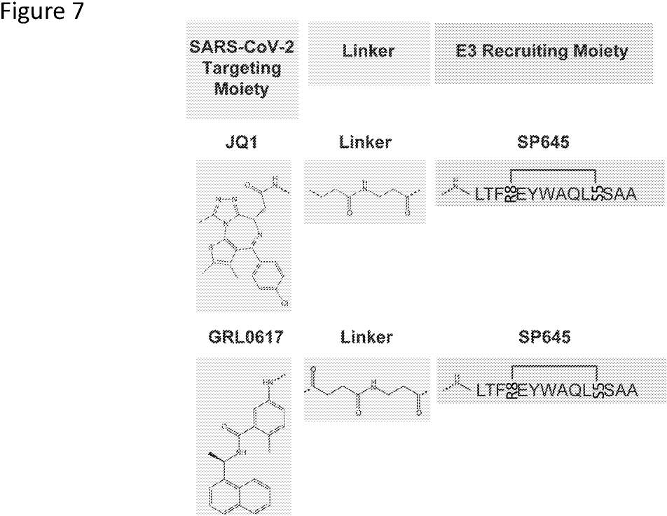

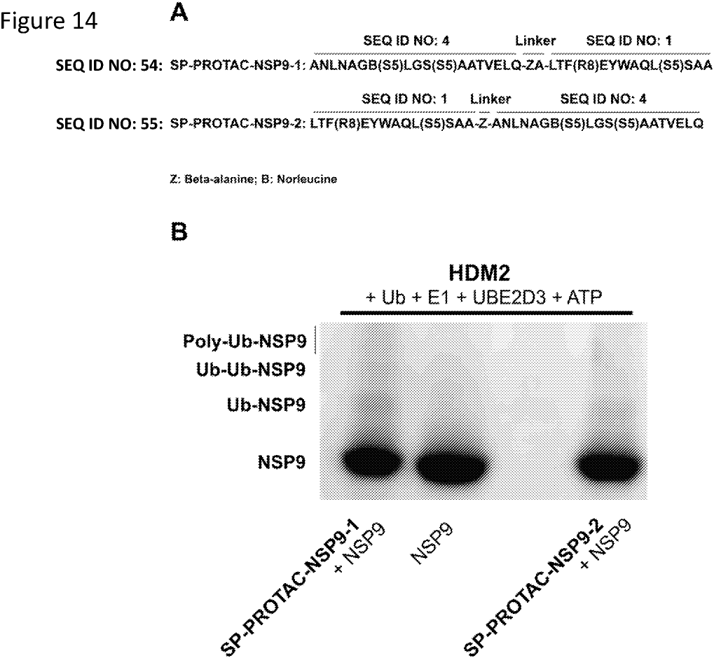

Non-limiting examples of the chimeras encompassed by this disclosure include each of the molecules shown in FIGS. 7 and 14A.

### I. First Protein Targeted for Degradation

The first protein targeted for degradation can be any protein that plays a role in viral pathogenesis. In some instances, the viral protein is an essential viral protein. In some instances, the protein targeted for degradation is a host protein that aids some activity of the virus.

### I.(a) Essential Viral Proteins

An essential viral protein that can be targeted with the chimeras of this disclosure includes any viral protein that plays a role in viral replication, and/or pathogenesis, including viral entry into a cell. In some embodiments, the essential viral protein is a viral protein from an RNA virus, such as Severe acute respiratory syndrome-associated coronavirus-2 SARS-CoV), SARS-CoV2, and Middle East Respiratory Syndrome-associated coronavirus (MERS-CoV). For instance, the viral protein is a NSP such as a coronaviral protease from SARS-CoV2. In some embodiments, the coronaviral protease is a PLpro or an Mpro. In some embodiments, the NSP is NSP9 or NSP12. In other embodiments, the essential viral protein is a viral protein from an RNA virus such as Hepatitis C Virus (HCV), Human immunodeficiency virus (HIV), Herpes simplex virus (HSV), Zika virus, and enterovirus.

### I.(a) Coronaviral Proteases

The coronaviral proteases described herein include Main protease (Mpro) and papain-like protease (PLpro), which are required for processing replicase polyproteins essential for the replication of coronavirus genomic RNA. These coronaviral proteases cleave the two translated viral polyproteins (PP1A and PP1AB) by extensive proteolytic processing into individual functional components in a coordinated manner (Chen Y.W. et al. F1000Research, 2020, 9:129).

As the main proteolytic processing enzyme of SARS-CoV-2, Mpro plays a major role in the autoprocessing proteolytic reactions that yield mature proteins (NSP5-16) essential to the viral life cycle (Chang, G.G. et al. Molecular Biology of the SARS-Coronavirus, 115-128 (2009)). The functional importance of Mpro (also known as 3C-like protease (3CLpro)) in viral replication, combined with the absence of closely related homologies in humans make Mpro an attractive target for the design of antiviral drugs.

PLpro is a multifunctional cysteine protease that processes the viral polyprotein and host cell proteins required for viral replication (Baez-Santos, Y.M., et al. J Virol 88, 12511-27 (2014)). PLpro is also involved in viral mechanisms for promoting p53 degradation (Ma-Lauer, Y. et al. Proc Natl Acad Sci USA 113, E5192-201 (2016)) and evading the host immune system (deuibiquitylating and deISGylating activity (Ratia, K., et al. PLoS Pathog 10, e1004113 (2014)). Therefore, targeting PLpro with antiviral drugs may have an advantage in not only inhibiting viral replication but also inhibiting the dysregulation of signaling cascades in infected cells that may lead to cell death in surrounding, uninfected cells.

### I.(a) Coronavirus Non-Structural Proteins

The coronaviral non-structural proteins (NSPs) described herein are essential proteins that can be targeted using the chimeras of this disclosure. These non-structural proteins include coronaviral proteases, NSP9 and NSP12. Coronaviral replication and transcription are driven by the 15 or 16 viral NSPs encoded in the replicase gene, any of which can be targeted by the chimeras of this disclosure. These NSPs are produced during co- and post-translational processing of the PP1a and PP1ab replicase polyproteins (te Velthuis, Aartjan JW. et al. Nucleic acids research 38,1 (2010): 203-14). The multi-subunit coronaviral RNA synthesis machinery is a complex of NSPs (Kirchdoerfer, R.N., Ward, A.B. Nat Commun 10, 2342 (2019)).

NSP9 is an essential protein of the viral replication complex. Its activity also depends on its dimerization mediated by parallel alpha-helices containing the protein-protein interaction motif GXXXG (SEQ ID NO: 8). NSP12 is the catalytic subunit of the coronaviral RNA replication complex, with RNA-dependent RNA polymerase (RdRp) activity. NSP12 possesses an architecture common to all viral polymerases and nucleotide analogs that block viral RNA replication.

### I.(a) Host Proteins That Facilitate Viral Pathogenesis

Another example of a protein that can be targeted with the chimeras of this disclosure can be a host protein that plays a role in viral pathogenesis. For instance, a host protein may be human protein such as a bromodomain and extraterminal domain (BET) protein. In some embodiments, the BET protein is bromodomain 2 (BRD2) or bromodomain 4 (BRD4). In other embodiments, the BET protein is bromodomain 3 (BRD3).

### II. First Moiety of Chimera

The first moiety of a chimera of this disclosure binds to a protein (e.g., a coronaviral protease) that is targeted for degradation. The first moiety can include a small molecule, a small molecule derivatized with a warhead, a peptide, a stapled peptide, a peptide or stapled peptide derivatized with a warhead, or a nucleotide analog. For instance, the first moiety can target a viral protein (e.g., an essential viral protein needed for viral replication or pathogenesis). In some cases, the first moiety can target a host protein that aids in viral pathogenesis. The first moiety can fully or partially act as a “molecular glue” which can bind to the protein targeted for degradation but itself does not necessarily have any inhibitory or agonistic effects.

In some embodiments, the first moiety is or includes an Mpro binder, such as an Mpro inhibitor. In other embodiments, the first moiety is or includes a PLpro binder, such as a PLpro inhibitor. In certain embodiments, the first moiety is or includes an NSP binder, such as an NSP inhibitor (e.g., an NSP9 or an NSP12 inhibitor). In other embodiments, the first moiety is or includes a BET binder, such as a BRD inhibitor (e.g., a BRD2, BRD3, or BRD4 inhibitor).

### II. (a) Mpro Binders

The Mpro binders encompassed by the present disclosure include agents that directly interact with Mpro, such as Mpro inhibitors. The Mpro inhibitors can inhibit Mpro dimerization and/or Mpro enzymatic activity. Mpro inhibitors such as those described in Jin Z et al. Nature 2020 582:289-293; Zumla A et al; Nat. Rev. Drug Disc. 2016 (10):327; Li G and Clercq ED. Nature Rev. Drug Disc., February 2020; Ghosh A.K. et al., ChemMedChem 2020 15(11): 907-932 can be used in the present disclosure.

In some embodiments, an Mpro binder is a peptide (e.g., a recombinant or synthetically produced peptide). Such peptides can be non-cross-linked, stapled, or stitched, so long as the peptides interact with Mpro as described herein. The enzymatic activity of Mpro relies on forming a homodimer mediated by an alpha-helical sequence: TVNVLAWLYAAVINGD (SEQ ID NO: 9). SEQ ID NO: 9 can be used to create peptide-based dimerization inhibitors. In some instances, Mpro binder peptides can include at least six (e.g., 7, 8, 9, 10, 11, 12, 13, 14, 15, or 16 contiguous amino acids of SEQ ID NO: 9). In some instances, the Mpro binder peptide can be a variant of SEQ ID NO: 9 - e.g., differing from SEQ ID NO: 9 by 1, 2, 3, 4, 5, or 6 amino acid substitutions, deletions, and/or insertions, wherein the variant can still dimerize with Mpro.

In some embodiments, Mpro binder peptides can comprise, consist, or consist essentially of the amino acid sequences of e.g., SEQ ID NO: 2 or 3. In some embodiments, the peptides can comprise, consist, or consist essentially of amino acid sequences related or with identity to a portion or portions of the amino acid sequence of e.g., SEQ ID NO: 2 or 3.

In some instances, the peptides can have at least or about 30%, at least or about 40%, at least or about 50%, at least or about 60%, at least or about 70%, at least or about 75% identity, at least or about 80%, at least 85%, at least or about 90%, at least or about 95%, at least or about 98%, at least or about 99%, or 100% identity to those amino acids in SEQ ID NO: 2 or 3, wherein the peptides bind to Mpro. Alternatively or in addition, the peptides can include amino acid substitutions and/or deletions, whether conservative or not. For example, the peptide can include 0, 1, 2, 3, 4, 5, 6, 7, 8, 9, less than 10, less than 5, less than 4, less than 3, or less than 2 amino acid substitutions, deletions, and/or additions, whether conservative or not, provided that the peptide can still bind to Mpro. Accordingly, the amino acid sequence of any of the Mpro binding peptides disclosed herein can be varied so long as the variant peptide binds to Mpro.

In certain embodiments, the peptides differ from the peptides of SEQ ID NO: 2 or 3 in that they vary from SEQ ID NO: 2 or 3 in having 1 to 4 (e.g., 1, 2, 3, 4) amino acid substitutions. For instance, the positions labeled “X” can be substituted in SEQ ID NO: 2 or 3 as follows: ATXNVLXWLYXAVIXGD (SEQ ID NO: 51). X can be a conservative or non-conservative amino acid residue. In some instances, each X is a non-natural amino acid with olefinic side chains (e.g., S5).

Exemplary Mpro binders that can be utilized in the chimeras described herein have the structures provided below:

TABLE 1

Mpro binders and their structures

| | |
|---|---|
| Mpro binder | Structure |
| Lopinavir (ABT-378) | 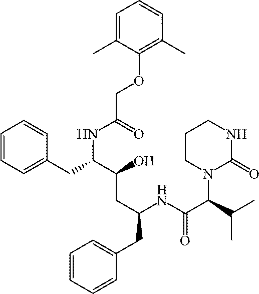 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O] |
| Ritonavir (Norvir) | 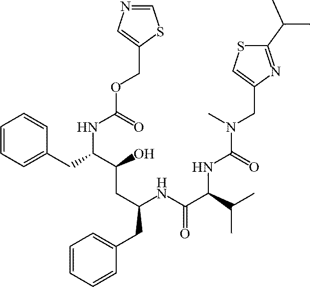 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1] |
| Darunavir | 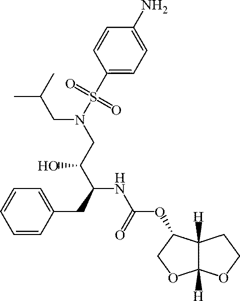 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1] |
| ASC09 (HIV protease inhibitor) | 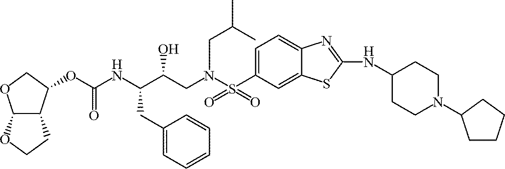 [SMILES: CC(C)CN(C[C@@H](O)[C@H](Cc1ccccc1)NC(=O)O[C@H]1CO[C@H]2OCC[C@H]21)S(=O)(=O)c1ccc2nc(NC3CCN(C4CCCC4)CC3)sc2c1] |
| GC376 (ANIV-19) | 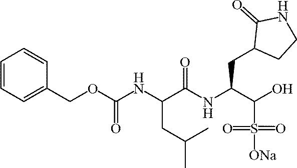 |
| GC813 | 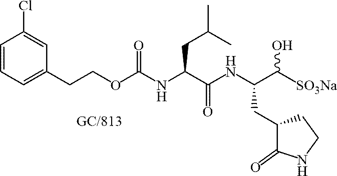 |
| Ebselen carboxylic acid | 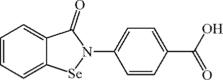 [SMILES: O=C(O)c1ccc(-n2[se]c3ccccc3c2=O)cc1] |
| N3 | 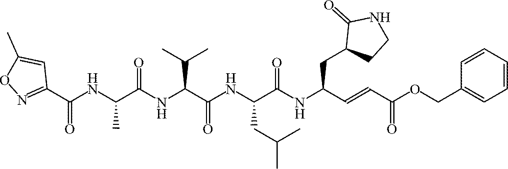 [SMILES: Cc1cc(C(=O)N[C@@H](C)C(=O)N[C@H](C(=O)N[C@@H](CC(C)C)C(=O)N[C@H](/C=C/C(=O)OCc2ccccc2)C[C@@H]2CCNC2=O)C(C)C)no1] |
| Tideglusib | 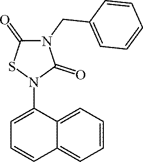 [SMILES: O=c1sn(-c2cccc3ccccc23)c(=O)n1Cc1ccccc1] |
| Carmofur | 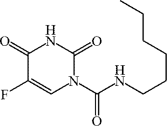 [SMILES: CCCCCCNC(=O)n1cc(F)c(=O)[nH]c1=O] |
| Shikonon | 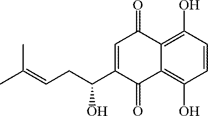 [SMILES: CC(C)=CC[C@@H](O)C1=CC(=O)c2c(O)ccc(O)c2C1=O] |
| α-ketoamide inhibitor 11r | 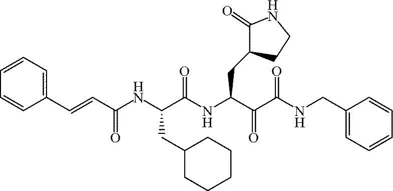 [SMILES: O=C(/C=C/c1ccccc1)N[C@@H](CC1CCCCC1)C(=O)N[C@@H](C[C@@H]1CCNC1=O)C(=O)C(=O)NCc1ccccc1] |
| α-ketoamide inhibitor 13a | 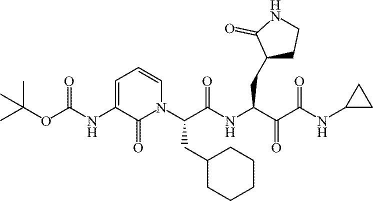 [SMILES: CC(C)(C)OC(=O)Nc1cccn([C@@H](CC2CCCCC2)C(=O)N[C@@H](C[C@@H]2CCNC2=O)C(=O)C(=O)NC2CC2)c1=O] |
| α-ketoamide inhibitor 13b | 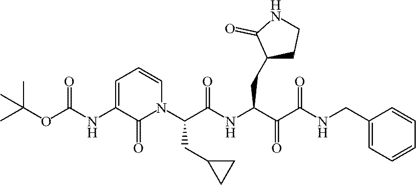 [SMILES: CC(C)(C)OC(=O)Nc1cccn([C@@H](CC2CC2)C(=O)N[C@@H](C[C@@H]2CCNC2=O)C(=O)C(=O)NCc2ccccc2)c1=O] |
| α-ketoamide inhibitor 14b | 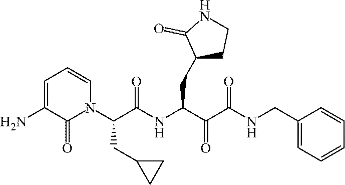 [SMILES: Nc1cccn([C@@H](CC2CC2)C(=O)N[C@@H](C[C@@H]2CCNC2=O)C(=O)C(=O)NCc2ccccc2)c1=O] |
| AG7088 | 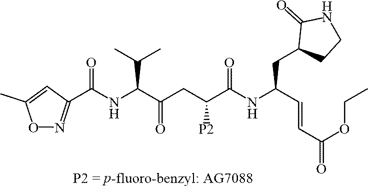 |

Any of the Mpro binders shown in Table 1 or Mpro-binding analogs thereof can be utilized in the chimeras of this disclosure.

### II.(b) PLpro Binders

The PLpro binders encompassed by the present disclosure include agents that directly interact with PLpro, such as PLpro inhibitors. The PLpro inhibitors can inhibit PLpro enzymatic activity. PLpro inhibitors such as those described in Baez-Santos, Y.M., et al. J Virol 88, 12511-27 (2014); Li G and Clercq ED. Nature Rev. Drug Disc., February 2020; Ghosh, AK. et al. J Med Chem 52, 5228-40 (2009); Baez-Santos, Y.M., et al. Antiviral Res 115, 21-38 (2015); Elfiky A and Ibrahim NS. Biophysics Feb. 11, 2020; article/rs-13849/v1 DOI: 10.21203/rs.2.23280/v1; Lin MH, et al. Antiviral Res. 2018;150:155-163 can be used in the chimeras of the present disclosure.

Exemplary PLpro binders that can be utilized in the chimeras described herein have the structures provided below:

TABLE 2

PLpro binders and their structures

| | |
|---|---|
| PLpro binders | Structure |
| GRL-0617 | 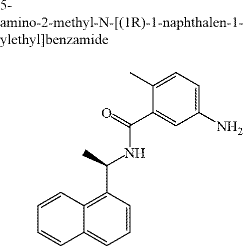 |
| GRL-0667 | 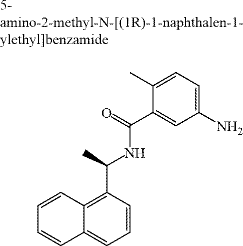 |
| Mycophenolic acid | 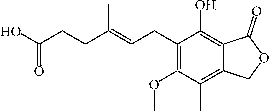 [SMILES: COc1c(C)c2c(c(O)c1C/C=C(\C)CCC(=O)O)C(=O)OC2] |
| Disulfiram | Tetraethylthiuram disulfide |
| | 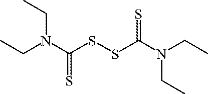 [SMILES: CCN(CC)C(=S)SSC(=S)N(CC)CC] |
| NSC158362 | 2-(Phenethylthio)acetic acid |
| | 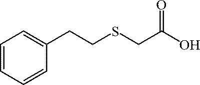 [SMILES: O=C(O)CSCCc1ccccc1] |
| NSC158011 | N-(1-Naphthyl)-2-(phenylthio)ethanethioamide |
| | 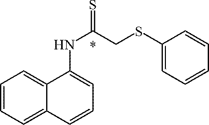 |
| Inhibitor 24 | 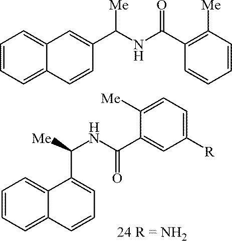 |
| Inhibitor 2 | 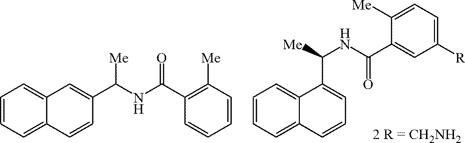 |
| Inhibitor 49 | 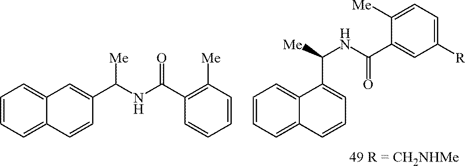 |
| 6 mercaptopurine (6MP, Purinethol, Purinax) | |
| | 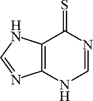 |
| 6 thioguanine (Tioguanine, 6TG, Lanvis, Tabloid) | 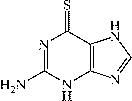 [SMILES: Nc1nc(=S)c2[nH]cnc2[nH]1] |
| Compound 7724772 | 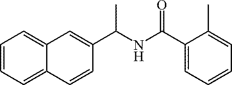 [SMILES: Cc1ccccc1C(=O)NC(C)c1ccc2ccccc2c1] |
| Compound 24 | 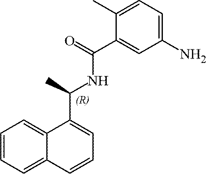 [SMILES: Cc1ccc(N)cc1C(=O)N[C@H](C)c1cccc2ccccc12] |
| Compound 6577871 | 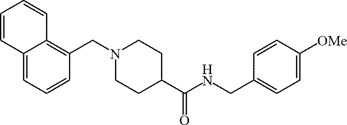 [SMILES: COc1ccc(CNC(=O)C2CCN(Cc3cccc4ccccc34)CC2)cc1] |
| Compound 15g | 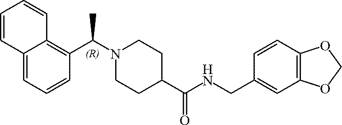 [SMILES: C[C@H](c1cccc2ccccc12)N1CCC(C(=O)NCc2ccc3c(c2)OCO3)CC1] |
| Compound 3k | 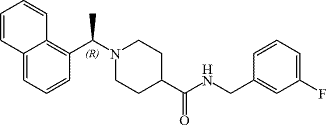 [SMILES: C[C@H](c1cccc2ccccc12)N1CCC(C(=O)NCc2cccc(F)c2)CC1] |
| Compound 3e | 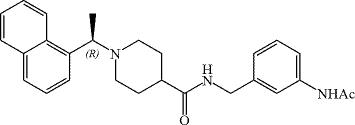 [SMILES: CC(=O)Nc1cccc(CNC(=O)C2CCN([C@H](C)c3cccc4ccccc34)CC2)c1] |
| Compound 3j | 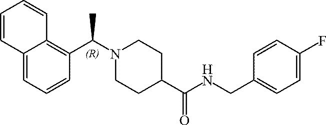 [SMILES: C[C@H](c1cccc2ccccc12)N1CCC(C(=O)NCc2ccc(F)cc2)CC1] |
| Compound 5c | 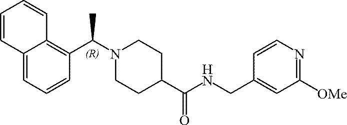 [SMILES: COc1cc(CNC(=O)C2CCN([C@H](C)c3cccc4ccccc34)CC2)ccn1] |

Any of the PLpro binders shown in Table 2 or PLpro-binding analogs thereof can be utilized in the chimeras of this disclosure.

### II.(c) NSP9 Binders

The NSP9 binders of the present disclosure are agents that can directly interact with NSP9. An NSP9 binder can be an NSP9 inhibitor which suppresses NSP9 dimerization and/or NSP9 enzymatic activity. In some embodiments, an NSP9 binder is a peptide (e.g., a recombinant or synthetically produced peptide). Such peptides can be non-cross-linked, stapled, or stitched, so long as the peptides interact with NSP9 as described herein. The enzymatic activity of NSP9 relies on forming a homodimer mediated by an alpha-helical sequence: NLNRGMVLGSLAATVRLQ (SEQ ID NO: 10). SEQ ID NO: 10 can be used to create peptide-based dimerization binders. In some instances, NSP9 binder peptides can include at least six (e.g., 7, 8, 9, 10, 11, 12, 13, 14, 15, 16, 17, or 18 contiguous amino acids of SEQ ID NO: 10). In some instances, NSP9 binder peptides can include at least 1, 2, 3, 4, 5, 6, 7, or 8 amino acid substitutions or deletions in SEQ ID NO: 10 so long as the variant peptide still binds NSP9. In some instances, an NSP9 binder peptide includes the sequence GXXXG (SEQ ID NO: 8), where each X can be any amino acid. In some instances X is any one M, norleucine (B), V, L, A, G, or I.

In some embodiments, NSP9 inhibitor peptides can comprise, consist, or consist essentially of the amino acid sequences of e.g., SEQ ID NO: 4 or 5. In some embodiments, the peptides can include amino acid sequences related or with identity to a portion or portions of the amino acid sequence of e.g., SEQ ID NO: 4 or 5.

In some instances, the peptides can have at least or about 30%, at least or about 40%, at least or about 50%, at least or about 60%, at least or about 70%, at least or about 80%, at least 85%, at least or about 90%, at least or about 95%, at least or about 98%, at least or about 99%, or 100% identity to those amino acids in SEQ ID NO: 4 or 5, wherein the peptides bind to NSP9. Alternatively or in addition, the peptides can include amino acid substitutions and/or deletions, whether conservative or not. For example, amino acids can include 0, 1, 2, 3, 4, 5, 6, 7, 8, less than 10, less than 5, less than 4, less than 3, or less than 2 amino acid substitutions, deletions, and/or additions, whether conservative or not. Accordingly, the amino acid sequence of any NSP9 binding peptide disclosed herein can be varied so long as the variant peptide can still binds to NSP9. In some instances, an NSP9 binder peptide of SEQ ID NO: 4 or 5 or variants thereof can be shortened by 1, 2, or 3 amino acids at each end of the sequence. In other instances, an NSP9 binder peptide of SEQ ID NO: 4 or 5 or variants thereof can include either no staple, one staple (e.g., a staple formed between R8 and S5), or be double stapled.

### 11. (D) NSP12 Binders

The NSP12 binders of the present disclosure include agents that directly interact with NSP12. An NSP12 binder can be an NSP12 inhibitor which suppresses NSP12 enzymatic activity. In some embodiments, an NSP12 inhibitor is a remdesivir or sofosbuvir analog (such as remdesivir acid or sofosbuvir carboxylic acid, or a pharmaceutically acceptable salt thereof).

The structure of remdesivir is as follows:

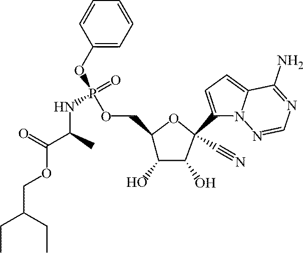 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

The structure of sofosbuvir is as follows:

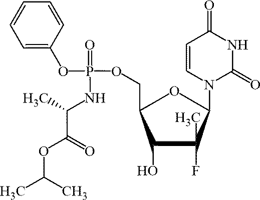

### II.(e) BET Binders

The BET binders of the present disclosure include agents that directly interact with a BET protein, such as bromodomain 2 (BRD2), BRD3 and/or BRD4. A BET protein binder can be a BET protein inhibitor which suppresses BET enzymatic activity. In some embodiments, BET inhibitors that can be utilized in the chimeras described herein have the structures provided below:

TABLE 3

BET protein inhibitors and their structures

| | |
|---|---|
| BET protein inhibitor | Structure |
| JQ1 | 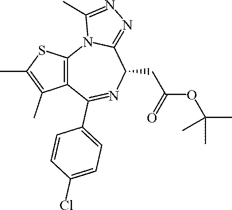 [SMILES: Cc1sc2c(c1C)C(c1ccc(Cl)cc1)=N[C@@H](CC(=O)OC(C)(C)C)c1nnc(C)n1-2] |
| ABBV-075 (Mivebresib) | 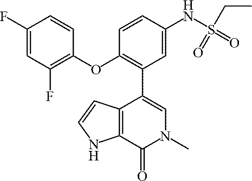 [SMILES: CCS(=O)(=O)Nc1ccc(Oc2ccc(F)cc2F)c(-c2cn(C)c(=O)c3[nH]ccc23)c1] |
| I-BET151 | 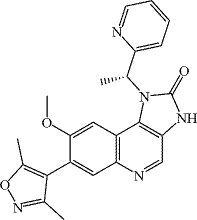 [SMILES: COc1cc2c(cc1-c1c(C)noc1C)ncc1[nH]c(=O)n([C@H](C)c3ccccn3)c12] |
| I-BET726 | 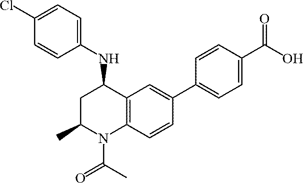 [SMILES: CC(=O)N1c2ccc(-c3ccc(C(=O)O)cc3)cc2[C@H](Nc2ccc(Cl)cc2)C[C@@H]1C] |
| OTX015 (Birabresib) | 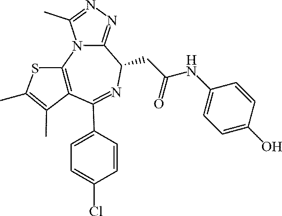 [SMILES: Cc1sc2c(c1C)C(c1ccc(Cl)cc1)=N[C@@H](CC(=O)Nc1ccc(O)cc1)c1nnc(C)n1-2] |
| PFI-1 | 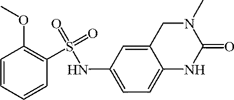 [SMILES: COc1ccccc1S(=O)(=O)Nc1ccc2c(c1)CN(C)C(=O)N2] |

Any of the BET inhibitors shown in Table 3 or BET-inhibiting analogs thereof can be utilized in the chimeras of this disclosure.

### III. Second Protein Targeted by Chimera

The second protein that is targeted with the chimeras of this disclosure is, or recruits, a protein degrader which degrades the first targeted protein (e.g., a coronaviral protease, a coronaviral NSP, or a BET protein). In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. Non limiting examples of E3 ubiquitin ligases include human double minute 2 (HDM2), Von Hippel-Lindau (VHL), Cereblon, X-linked inhibitor of apoptosis protein (XIAP), cellular inhibitor of apoptosis protein (cIAP), or Constitutive photomorphogenic 1 (COP1).

### IV. Second Moiety of Chimera

The second moiety of a chimera of this disclosure binds to a protein that is or recruits “a protein degrader” (e.g., an E3 ubiquitin ligase) that degrades the target protein (e.g., a coronaviral protease, a coronaviral NSP, or a BET protein). In some embodiments, the second moiety can recruit an enzyme or a complex that catalyzes the ubiquitination of the target. An example of a “protein degrader” is an E3 ligase that ubiquitylates the target protein, thereby labeling that target protein for degradation by a proteasome.

The second moiety can be a peptide, a stapled peptide, or a small molecule. In some embodiments, the second moiety is or includes a cereblon binding moiety, a VHL binding moiety, an HDM2 binding moiety, an XIAP binding moiety, a cIAP binding moiety, or a COP1 binding moiety.

### IV.(a) Cereblon Binding Moiety

The second moiety of the chimera of this disclosure can be a cereblon binding moiety that is or includes a small molecule. Non-limiting examples of small molecules that bind to cereblon include but are not limited to thalidomide, pomalidomide, lenalidomide, avadomide, analogs thereof, and pharmaceutically acceptable salts thereof.

The small molecules that can bind to cereblon have the following structures:
Thalidomide:

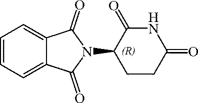 [SMILES: O=C1CC[C@@H](N2C(=O)c3ccccc3C2=O)C(=O)N1]

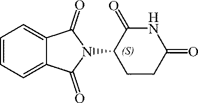 [SMILES: O=C1CC[C@H](N2C(=O)c3ccccc3C2=O)C(=O)N1]

Pomalidomide:

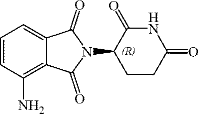 [SMILES: Nc1cccc2c1C(=O)N([C@@H]1CCC(=O)NC1=O)C2=O]

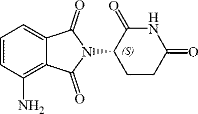 [SMILES: Nc1cccc2c1C(=O)N([C@H]1CCC(=O)NC1=O)C2=O]

Lenalidomide:

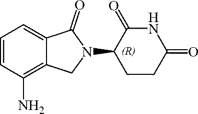 [SMILES: Nc1cccc2c1CN([C@@H]1CCC(=O)NC1=O)C2=O]

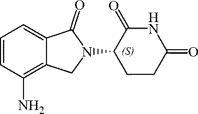 [SMILES: Nc1cccc2c1CN([C@H]1CCC(=O)NC1=O)C2=O]

Avadomide:

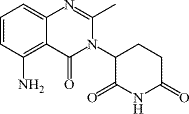 [SMILES: Cc1nc2cccc(N)c2c(=O)n1C1CCC(=O)NC1=O]

In one embodiment, the small molecule that binds to cereblon is based on a thalidomide having the structure provided below:

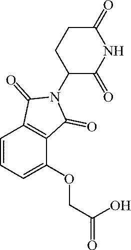 [SMILES: O=C(O)COc1cccc2c1C(=O)N(C1CCC(=O)NC1=O)C2=O]

In some embodiments, the cereblon binding moiety is conjugated to the N-terminus of the first protein-targeting stapled peptide. In some instances, the cereblon moiety is conjugated to the C-terminus of the first protein-targeting stapled peptide. In certain instances, the cereblon moiety is contained within a non-natural amino acid inserted in the peptide sequence between the N- and C-terminus of the first protein-targeting stapled peptide.

In some instances, the small molecule (thalidomide) that binds to cereblon can be conjugated at the N-terminus of the first protein-targeting moiety (e.g., a peptide). When the small molecule (thalidomide) is conjugated at the N-terminus of a peptide, it has the structure shown below:

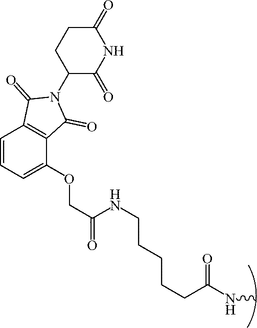

In certain instances, when the small molecule (thalidomide) is conjugated at the C-terminus of a first protein-targeting peptide, it has the structure shown below:

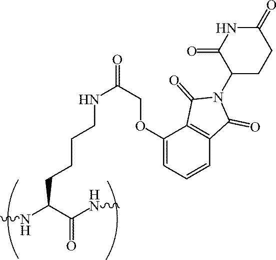

In certain instances, the cereblon-binding moiety is contained within a non-natural amino acid inserted in the peptide sequence between the N- and C-terminus of the first protein-targeting stapled peptide.

### IV.(b) VHL Binding Moiety

The second moiety of the chimera of this disclosure can be a VHL binding moiety that is or comprises a small molecule. Non-limiting examples of small molecules that bind to VHL include but are not limited to VH 032 and VHL-binding analogs thereof.

The small molecules that can bind to VHL have the following structures: VH 032:

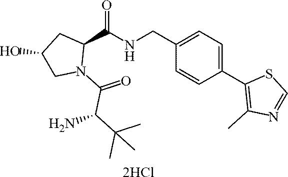

In some instances, when the VHL moiety is conjugated to an amine, the carboxylate analog pictured below is employed:

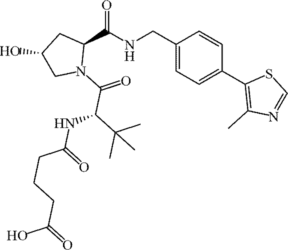 [SMILES: Cc1ncsc1-c1ccc(CNC(=O)[C@@H]2C[C@@H](O)CN2C(=O)[C@@H](NC(=O)CCCC(=O)O)C(C)(C)C)cc1]

In some instances, the VHL moiety comprises the structure below (compatible with coupling to acid residues):

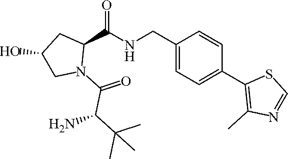 [SMILES: Cc1ncsc1-c1ccc(CNC(=O)[C@@H]2C[C@@H](O)CN2C(=O)[C@@H](N)C(C)(C)C)cc1]

In some embodiments, the VHL binding moiety is conjugated to the N-terminus of the first protein-targeting stapled peptide. In some instances, the VHL moiety is conjugated to the C-terminus of the first protein-targeting stapled peptide. In certain instances, the VHL moiety is contained within a non-natural amino acid inserted in the peptide sequence between the N- and C-terminus of the first protein-targeting stapled peptide.

In certain instances, the VHL binding moiety is a peptide that has the amino acid sequence of a peptide listed below, or a VHL-binding variant thereof:

LAPAAGDTIISLDF (SEQ ID NO: 12)

- E3 ligase: VHL;

LAPYIPMDDDFQL (SEQ ID NO: 13)

- E3 ligase: VHL.

The variant can differ from SEQ ID NO: 12 or 13 at e.g., 1, 2, 3, 4, 5, or 6 amino acid positions (e,g., by substitution with another amino acid, insertion, or deletion), provided it still binds VHL.

### IV.(c) HDM2 Binding Moiety

The second moiety of the chimera of this disclosure can be an HDM2 binding moiety that is or comprises a small molecule, a peptide or a stapled peptide or an otherwise chemically-stabilized peptide (e.g., stitched) of the transactivation domain of p53 that binds HDM2 and/or HDMX. Non-limiting examples of small molecules that bind to HDM2 include but are not limited Nutlin-3, Nutlin-3a, Nutlin-3a derivatives such as RG7112 and RG7388 (Idasanutlin), and HDM2-binding analogs thereof.
Nutlin-3a has the following structure:

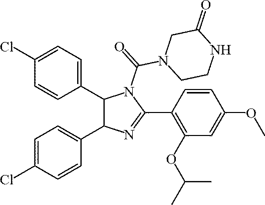 [SMILES: COc1ccc(C2=NC(c3ccc(Cl)cc3)C(c3ccc(Cl)cc3)N2C(=O)N2CCNC(=O)C2)c(OC(C)C)c1]

RG7112 has the following structure:

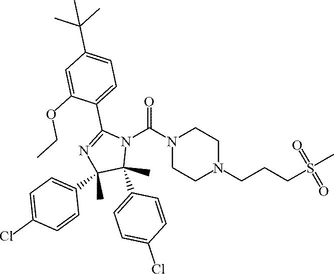 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

RG7388 has the following structure:

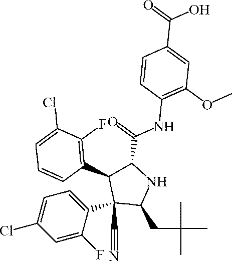 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

In certain embodiments, the HDM2 binding moiety is a p53 peptide or a stapled p53 peptide known in the art (e.g., a peptide comprising the transactivation domain of p53 (LSQETFSDLWKLLPEN (SEQ ID NO: 11)), a variant thereof that binds HDM2, or a stapled version thereof). In certain instances, the HDM2 binding moiety is a stabilized or stapled p53 peptide that directly binds to and recruits a complex between HDMX and the HDM2. In some embodiments, a stapled p53 peptide can bind HDMX and recruit the HDMX/HDM2 complex. In certain embodiments, a stapled p53 peptide binds, directly or indirectly, to HDM2, or to HDMX which binds to HDM2.

In some instances, the HDM2-binding stapled peptides of the disclosure have the residues (R5), (R8), (S5), and (S8). In such sequences (e.g., SEQ ID NOs.: 1, 7, and 40-47), “R” and “S” refers to the stereochemistry of the non-natural amino acids, while “5” and “8” refer to the number of carbon residues in the olefinic side chains of the non-natural amino acid. Thus, “(R5)” is (R)-2-(4′-pentenyl)alanine [also known as (R)-α-(4′- pentenyl)alanine]; “(R8)” is (R)-2-(7′-octenyl)alanine [also known as (R)-α-(7′-octenyl)alanine]; “(S5)” is (S)-2-(4′-pentenyl)alanine [also known as (S)-α-(4′- pentenyl)alanine]; and “(S8)” is (S)-2-(7′-octenyl)alanine [also known as (S)-α-(7′-octenyl)alanine]. In some instances, SEQ ID NOs.: 1, 7, and 40-47 can be varied such that (R8) is replaced by (R5) and concurrently, (S5) is replaced by (S8). Such variant HDM2-binding stapled peptides are encompassed by this disclosure. These variants of SEQ ID NOs. 1, 7, and 40-47 retain the ability to bind to HDM2. For example, a variant sequence of SEQ ID NO: 1 is SEQ ID NO: 49; and a variant sequence of SEQ ID NO: 7 is SEQ ID NO: 50.

Non-limiting examples of stapled p53 peptides that may be used in the chimeras of this disclosure are listed below:

LTF(R8)EYWAQL(S5)SAA (SEQ ID NO: 1)

- SP645

LTF(R8)EYWAQ#(S5)SAA (SEQ ID NO: 7)

- ATSP-7041# is cyclobutylalanine,

LTF(R5)EYWAQL(S8)SAA (SEQ ID NO: 49)

- SP645 variant sequence 2

LTF(R5)EYWAQ#(S8)SAA (SEQ ID NO: 50)

- ATSP-7041 variant sequence

LSQETFSD(R8)WKLLPE(S5) (SEQ ID NO: 40)

- SAH-p53-1

LSQE(R8)FSDLWK(S5)LPEN (SEQ ID NO: 41)

- SAH-p53-2

LSQ(R8)TFSDLW(S5)LLPEN (SEQ ID NO: 42)

- SAH-p53-3

LSQETF(R8)DLWKLL(S5)EN (SEQ ID NO: 43)

- SAH-p53-4

LSQETF(R8)NLWKLL(S5)QN (SEQ ID NO: 44)

- SAH-p53-5

LSQQTF(R8)NLWRLL(S5)QN (SEQ ID NO: 45)

- SAH-p53-6

QSQQTF(R8)NLWKLL(S5)QN (SEQ ID NO: 46)

- SAH-p53-7

QSQQTF(R8)NLWRLL(S5)QN (SEQ ID NO: 47)

- SAH-p53-8

Other examples of stapled p53 peptides and other peptides that bind to HDM2 and/or HDMX and can be used in the chimeras of this disclosure include but are not limited to ALRN-6924, and the stapled or stitched p53 peptides provided in U.S. Pat. Nos.10,202,431; 9,617,309; 9,556,227; 9,527,896; 9,517,252; 9,505,804; 9,505,801; 9,408,885; 9,175,045; 9,163,330; 8,927,500; 8,889,632; 8,592,377; 8,586,707; U.S. Pat. Application Publication Nos. US20140018302; US20150246946; 20170212125; US20180030090; 20190076504; International Patent Application No. WO2008106507; WO2014065760; and European Patent Application No. EP2912463; the contents of all of which are incorporated by reference in their entirety herein.

In some instances, variants of any one of SEQ ID NOs.: 1, 7, 40-47, and 49-50 that still bind HDM2 and/or HDMX are encompassed by this disclosure. These variants can differ from any one of SEQ ID NOs.: 1, 7,40-47 and 49-50 at 1, 2, 3, 4, 5, or 6 amino acid positions (e.g., by substitution with a different amino acid, insertion, or deletion) wherein the variants retains its ability to bind HDM2. In one example, the tryptophan (“W”) residue in SEQ ID NO: 1 or 7 can be substituted by 3-(2-Naphthyl)-L-alanine). In certain embodiments, a variant differs from the peptide of SEQ ID NO: 1 or 7 in that it varies from SEQ ID NO: 1 or 7 in having 1 to 6 (e.g., 1, 2, 3, 4, 5, 6) amino acid substitutions. For instance, the residues labeled X₁, X₂, X₃, X₄, and X₅” can be substituted in SEQ ID NO: 1 or 7 as follows: X₁TF(R8)X₂YX₃AQX₄(S5)X₅AA (SEQ ID NO: 48) where X₁, X₂, and X₅ are any amino acid (e.g., A, W, F, L, V, I, naphthylalanine, E, D, Y, cyclobutylalanine and side chain analogs thereof); X₃: W, F, or 3-(2-naphthyl)-L-alanine; and X₄: L or a leucine mimetic (e.g., cyclobutylalanine).

In some instances, the peptides can have at least or about 30%, at least or about 40%, at least or about 50%, at least or about 60%, at least or about 70%, at least or about 75% identity, at least or about 80%, at least 85%, at least or about 90%, at least or about 95%, at least or about 98%, at least or about 99%, or 100% identity to those amino acids in SEQ ID NO: 1 or 7, wherein the peptides bind to HDM2 and/or HDMX. Alternatively or in addition, the peptides can include amino acid substitutions and/or deletions, whether conservative or not. For example, the peptide can include 0, 1, 2, 3, 4, 5, 6, less than 6, less than 5, less than 4, less than 3, or less than 2 amino acid substitutions, deletions, and/or additions, whether conservative or not, provided that the peptide can still bind to HDM2 and/or HDMX. Accordingly, the amino acid sequence of any of the HDM2 and/or HDMX peptides disclosed herein can be varied so long as the variant peptide binds to HDM2 and/or HDMX.

In some instances, the transactivation domain of p53 (LSQETFSDLWKLLPEN (SEQ ID NO: 11)), a variant thereof that binds HDM2, or a stabilized (e.g., stapled or stitched) form thereof can be used in the chimera of this disclosure. In some cases, the bolded F, W, and L of the above sequence are not substituted. In some cases, one or more of the bolded F, W, and L of the above sequence are only substituted by way of a conservative substitution. In certain instances, one or more aspartic acid (D) of the above sequence is substituted with asparagine. In certain instances, one or more glutamic acid (E) of the above sequence is substituted with glutamine. In some cases, the lysine of the above sequence is substituted with arginine. In some cases, the first leucine of the above sequence is substituted with glutamine. In some cases, the serine following phenylalanine and/or the threonine preceding the phenylalanine is/are substituted by any amino acid (e.g., a non-natural amino acid such as R8). In some cases, the leucine following lysine and/or the proline is/are substituted by any amino acid (e.g., a non-natural amino acid such as S5). In some cases, the first, second third, fourth and/or fifth N-terminal amino acids of SEQ ID NO: 11 may be deleted. In some cases, one, two, and/or three of the C-terminal amino acids of SEQ ID NO: 11 may be deleted. In certain cases the first, second third, fourth and/or fifth N-terminal amino acids and one, two, and/or three of the C-terminal amino acids of SEQ ID NO: 11 may be deleted. It is to be understood that a combination of one or more of these substitutions are encompassed by this disclosure. In some cases, the peptide or stabilized peptide is neutral or positively charged. In all cases, the peptide or stabilized peptide binds HDM2 (e.g., with nanomolar affinity). In some instances, the peptide or stapled peptide is 8, 9, 10, 11, 12, 13, 14, or 15, amino acids in length.

In certain instances, the second moiety peptide has the amino acid sequence of one of the peptides listed below, or HDM2-binding variants thereof (variations may be made as described in the preceding paragraph), such as those described in Chong Li et al., J Mol Biol. 2010 Apr 30; 398(2): 200-213:

FSDLWKLL (SEQ ID NO: 38)

- p53 transactivation domain sequence;

TSFAEYWNLLSP (SEQ ID NO: 39)

- PMI duodecimal peptide inhibitor.

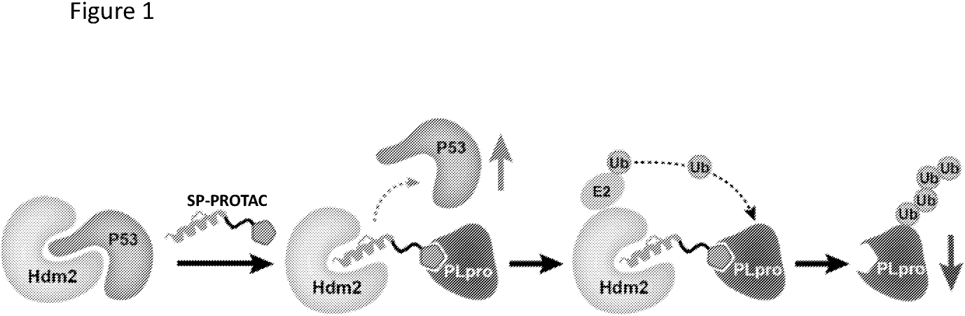

Other examples of p53 peptides that can be employed in the chimeras described herein are disclosed in WO 2017/165617 (FIG. 7) and WO 2012/065181 (FIG. 1C), the contents of all of which are incorporated by reference in their entireties (especially the disclosure of stapled and non-stapled p53 peptides).

Stapled p53 peptides can be modified in order to enhance their structural stability, peptide solubility, cell permeability, and associated degradation efficacy of the chimeras disclosed herein, into which the stapled p53 peptides are incorporated. For instance, p53 peptides can be modified in order to increase, α-helical content, retain high binding affinity for HDM2, enhance cellular uptake, etc. Some residues in the p53 peptides can be substituted to achieve the appropriate charge. For instance, aspartic and glutamic acids in the stapled p53 peptides can be replaced with asparagines and glutamines to adjust peptide charge, i.e., change the peptide charge from negative to neutral or positive to enhance cell penetration. Certain amino acid residues in p53 peptides can also be mutated in order to avoid nuclear export (L14Q) and/or avoid potential ubiquitylation (K24R).

F19, W23, and L26 are key human p53 residues for interacting with HDM2. Raj, N. and Attardi, L.D. Cold Spring Harb Perspect Med. 2017 Jan; 7(1): a026047. A stapled p53 peptide that can be used in a chimera of the disclosure includes all or part of transactivation domain sequences corresponding to amino acids 14-29 of human p53 (UniProtKB - P04637), or a variant thereof (e.g., and at least the essential interacting amino acids F19, W23, and L26) and completely or partially inhibits the binding of p53 to HDMX, HDM2, or HDMX and HDM2, as measured in an in vitro binding assay. The human wild-type amino acid sequence of the p53 transactivation domain that engages HDM2 and HDMX includes:

LSQETFSDLWKLLPEN (SEQ ID NO: 11)

which corresponds to amino acids 14-29 of full length p53.

Any or all amino acids of the stapled p53 peptide except for the essential interacting amino acids (see above) can be substituted, and/or one or more of the essential interacting amino acids (see above) can be substituted with one or more conservative substitutions. See, e.g., Coffill et al Genes Dev 2016 30: 281-292 and Baek at el JACS 2012 13: 103-6. In some cases, the first, second third, fourth and/or fifth N-terminal amino acids of SEQ ID NO: 11 may be deleted. In some cases, one, two, and/or three of the C-terminal amino acids of SEQ ID NO: 11 may be deleted. In certain cases the first, second third, fourth and/or fifth N-terminal amino acids and one, two, and/or three of the C-terminal amino acids of SEQ ID NO: 11 may be deleted.

A “non-essential” amino acid residue is a residue that can be altered from the wildtype and/or fully functional sequence of a polypeptide (without abolishing or substantially altering its activity). An “essential” amino acid residue is a residue that, when altered from the wild-type and/or fully functional sequence of the polypeptide, results in abolishing or substantially abolishing the polypeptide activity.

The term “essential” amino acid residue as used herein, includes conservative substitutions of the essential amino acid. Generally, the “essential” amino acid residues are found at the interacting face of the alpha helix.

Conservative substitutions suitable for inclusion in the stapled p53 peptides disclosed herein are discussed below can include substitutions in which one amino acid residue is replaced with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In one example of a conservative amino acid substitution, the essential amino acid phenylalanine (F19) in SEQ ID NO: 11, can be replaced with alanine.

### IV.(d) XIAP Binding Moiety

The second moiety of the chimera of this disclosure can be an XIAP binding moiety that is or comprises a small molecule. Non-limiting examples of small molecules that bind to XIAP include but are not limited to A410099.1 or an XIAP-binding analog thereof

The XIAP binding moiety A410099.1 has the following structure:

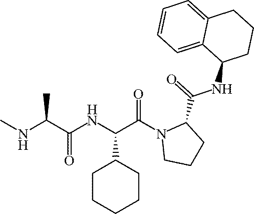 [SMILES: CN[C@@H](C)C(=O)N[C@H](C(=O)N1CCC[C@H]1C(=O)N[C@@H]1CCCc2ccccc21)C1CCCCC1]

### IV.(e) cIAP Binding Moiety

The second moiety of the chimera of this disclosure can be a cIAP binding moiety that is or comprises a small molecule. Non-limiting examples of small molecules that bind to cIAP include but are not limited to SM-1295 or SM-1280 or a cIAP-binding analog thereof. The cIAP binding moiety SM-1295 has the following structure:

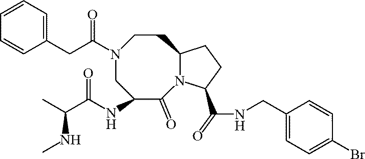 [SMILES: CN[C@@H](C)C(=O)N[C@H]1CN(C(=O)Cc2ccccc2)CC[C@H]2CC[C@@H](C(=O)NCc3ccc(Br)cc3)N2C1=O]

The cIAP binding moiety SM-1280 has the following structure:

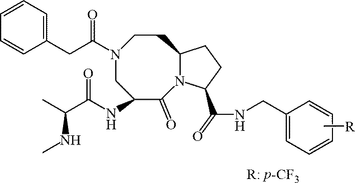

### IV.(f) COP1 Binding Moiety

The second moiety of the chimera of this disclosure can be a COP1 binding moiety that is or comprises a peptide or stapled peptide. Non-limiting examples of a peptide that binds to COP1 includes but is not limited to the Tribbles Pseudokinase 1 (Trib1) peptide with the following sequence: DQIVPEY (SEQ ID NO: 6) or variants thereof.

In some instances, the peptides can have at least or about 30%, at least or about 40%, at least or about 50%, at least or about 60%, at least or about 70%, at least or about 75% identity, at least or about 80%, at least 85%, at least or about 90%, at least or about 95%, at least or about 98%, at least or about 99%, or 100% identity to those amino acids in SEQ ID NO: 6, wherein the peptides bind to COP1. Alternatively or in addition, the peptides can include amino acid substitutions and/or deletions, whether conservative or not. For example, the peptide can include 0, 1, 2, 3, 4, 5, less than 5, less than 4, less than 3, or less than 2 amino acid substitutions, deletions, and/or additions, whether conservative or not, provided that the peptide can still bind to COP1. Accordingly, the amino acid sequence of any of the COP1 binding peptides disclosed herein can be varied so long as the variant peptide binds to COP1.

### IV.(g) Substrate Adaptor Binding Peptides

The second moiety of the chimera of this disclosure can be a peptide that binds a protein that is the substrate adaptor for an E3 ubiquitin ligase. In some instances, the peptide binds a WD-40 protein that is the substrate adaptor for an E3 ubiquitin ligase. The peptide binds to the substrate recognition domain of the E3 ubiquitin ligase in a shallow groove and is tolerant of elaboration (i.e., conjugation of a stapled peptide sequence) at either the N- or C-terminus. Exemplary substrate adaptors for an E3 ubiquitin ligase include HDM2, and VHL.

In certain instances, the second moiety peptides are based on the Tribbles Pseudokinase 1(Trib1) protein sequence: DQIVPEY (SEQ ID NO: 6) or variants thereof.

In certain instances, the second moiety peptides are based on a natural binding consensus sequence of a peptide that binds a WD40-repeat protein that is the substrate adaptor for an E3 ubiquitin ligase. In some cases the second moiety peptides are variants (e.g., substitution, deletion, or insertion variants) of the natural binding consensus sequence of a peptide that binds a WD40-repeat protein that is the substrate adaptor for an E3 ubiquitin ligase. Non-limiting examples of natural binding consensus sequence of a peptide that binds a WD40-repeat protein that is the substrate adaptor for an E3 ubiquitin ligase are provided below:

| SEQ ID NO: | E3 ligase/degron | No. of known instances | Motif pattern^{†‡} |
|---|---|---|---|
| 14 | MDM2_SWIB | 5 | F[^P] {3}W[^P] {2,3} [VIL] |
| 15 | ODPH_VHL_1 | 8 | [IL]A(P). {6,8} [FLIVM]. [FLIVM] |

†The motif pattern uses the following nomenclature: ‘.’ specifies any amino acid type, ‘[X]’ specifies the allowed amino acid type(s) at that position, ‘^X’ at the beginning of the pattern specifies that the sequence starts with amino acid type X, ‘[^X]’ denotes that the position can have any amino acid other than type X, numbers specified as the following ‘X{x,y}’, where x and y specify the minimum and maximum number of ‘X’ amino acid type required at that position. Conserved residue positions within the primary degron that are known to be post-translationally modified (for example, phosphorylation and proline hydroxylation) are shown in boldface.

### V. Second Moiety Peptides

The chimeras described herein can comprise an E3 ubiquitin ligase binding peptide (e.g., SEQ ID NOs.: 1, 6, 7, 12, 13, 38-47, and 49-50) as a second moiety. The second moiety peptide can be a stabilized or stapled peptide. Any other peptides known in the art can also be incorporated as a second moiety into the chimeras of this disclosure. See, e.g., Mészáros et al., Sci. Signal., 10(470):eaak9982 (2017); Guharoy et al., Nature Communications, 7:10239, doi:10.1038/ncomms10239 (2016); U.S. Pat. Nos. 9,783,575; 9,297,017; and 9,115,184, all of which are incorporated by reference herein in their entireties.

Variants of the peptides of this disclosure (i.e., SEQ ID NOs.: 1, 6, 7, 12, 13, 38-47, and 49-50) include peptides with one or more (e.g., 1, 2, 3, 4, 5) amino acid substitutions; one or more deletions (e.g., 1, 2, 3); one or more insertions (e.g., 1, 2, 3); or a combination of any two or more thereof. The variants that interact with the relevant E3 ligase are selected.

A second moiety peptide of this disclosure can bind its relevant E3 ligase with a binding affinity lower than 1000 nM. In certain instances, the second moiety peptide is 4 to 20 amino acids in length (e.g., 4, 5, 6, 7, 8, 9, 10, 11, 12, 13, 14, 15, 16, 17, 18, 19, or 20).

In certain instances, the second moiety peptide has the amino acid sequence of a peptide set forth in any one of SEQ ID NOs.: 1, 6, 7, 12, 13, 38-47, and 49-50. In some instances, the second moiety peptide has an amino acid sequence that is a variant of a peptide set forth in any one of SEQ ID NOs.: 1, 6, 7, 12, 13, 38-47, and 49-50. Variants include second moiety peptide with one or more (e.g., 1, 2, 3, 4, 5) amino acid substitutions; one or more deletions (e.g., 1, 2, 3); one or more insertions (e.g., 1, 2, 3); or a combination of any two or more thereof.

### VI. Stabilized Peptides

The first and/or second moiety of the chimeras of this disclosure can include stabilized peptides (e.g., a stapled peptides). A peptide helix is an important mediator of key protein-protein interactions that regulate many important biological processes (e.g., apoptosis); however, when such a helix is taken out of its context within a protein and prepared in isolation, it can unfold and adopt a random coil conformation, leading to a drastic reduction in biological activity and thus diminished therapeutic potential. To avoid this problem, one can employ structurally stabilized peptides. In some cases, structurally stabilized peptides comprise at least two modified amino acids joined by an internal (intramolecular) cross-link (or staple). Stabilized peptides as described herein include stapled peptides, stitched peptides, peptides containing multiple stitches, peptides containing multiple staples, or peptides containing a mix of staples and stitches, as well as peptides structurally reinforced by other chemical strategies (see. e.g., Balaram P. Cur. Opin. Struct. Biol. 1992;2:845; Kemp DS, et al., J. Am. Chem. Soc. 1996;118:4240; Orner BP, et al., J. Am. Chem. Soc. 2001;123:5382; Chin JW, et al., Int. Ed. 2001;40:3806; Chapman RN, et al., J. Am. Chem. Soc. 2004;126:12252; Horne WS, et al., Chem., Int. Ed. 2008;47:2853; Madden et al., Chem Commun (Camb). 2009 Oct 7; (37): 5588-5590; Lau et al., Chem. Soc. Rev., 2015,44:91-102; and Gunnoo et al., Org. Biomol. Chem., 2016,14:8002-8013; all of which are incorporated by reference herein in their entirety). For more examples of stabilized peptides, see e.g., WO 2019/118893, which is hereby incorporated by reference in its entirety.

In certain embodiments, polypeptides can be stabilized by peptide stapling (see, e.g., Walensky, J. Med. Chem., 57:6275-6288 (2014), the contents of which are incorporated by reference herein in its entirety). Stapled peptides reinforce the natural α-helical shape of bioactive peptides, conferring stabilized structure, protease resistance in vivo, enhanced target binding affinity, and favorable pharmacology (Walensky, L.D. & Bird, G.H. J Med Chem 57, 6275-88 (2014); Walensky, L.D. et al. Science 305, 1466-70 (2004)). A peptide is “stabilized” in that it maintains its native secondary structure. For example, stapling allows a polypeptide, predisposed to have an α-helical secondary structure, to maintain its native α-helical conformation. This secondary structure increases resistance of the polypeptide to proteolytic cleavage and heat, and also may increase target binding affinity, hydrophobicity, and cell permeability. Accordingly, the stapled (cross-linked) polypeptides described herein have improved biological activity relative to a corresponding non-stapled (un-cross-linked) polypeptide.

“Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacing. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced.

In certain embodiments, polypeptides can be stabilized by, e.g., hydrocarbon stapling. In certain instances, the stapled peptide includes at least two (e.g., 2, 3, 4, 5, 6) amino acid substitutions, wherein the substituted amino acids are separated by two, three, or six amino acids, and wherein the substituted amino acids are non-natural amino acids with olefinic side chains. There are many known non-natural or unnatural amino acids any of which may be included in the stapled peptides. Some examples of unnatural amino acids are 4-hydroxyproline, desmosine, gamma-aminobutyric acid, beta-cyanoalanine, norvaline, 4-(E)-butenyl-4(R)-methyl-N- methyl-L-threonine, N-methyl-L-leucine, 1-amino-cyclopropanecarboxylic acid, 1- amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and /para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C═O)C₆H₅; —CF₃; —CN; -halo; —NO₂; CH₃), and statine. Additionally, amino acids can be derivatized to include amino acid residues that are hydroxylated, phosphorylated, sulfonated, acylated, or glycosylated.

Hydrocarbon stapled polypeptides include one or more tethers (linkages) between two non-natural amino acids, which tether significantly enhances the α-helical secondary structure of the polypeptide. Generally, the tether extends across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . X1, X2, X3, X4, X5, X6, X7, X8, X9 ..., cross-links between X1 and X4, or between X1 and X5, or between X1 and X8 are useful hydrocarbon stapled forms of that peptide, as are cross-links between X2 and X5, or between X2 and X6, or between X2 and X9, etc. The use of multiple cross-links (e.g., 2, 3, 4, or more) is also contemplated. The use of multiple cross-links is very effective at stabilizing and optimizing the peptide, especially with increasing peptide length. Thus, the peptides which can be incorporated into the chimeras of this disclosure can incorporate more than one cross-link within the polypeptide sequence to either further stabilize the sequence or facilitate the structural stabilization, proteolytic resistance, acid stability, thermal stability, cellular permeability, and/or biological activity enhancement of longer polypeptide stretches. Additional description regarding making and use of hydrocarbon stapled polypeptides can be found, e.g., in U.S. Pat. Publication Nos. 2012/0172285, 2010/0286057, and 2005/0250680, the contents of all of which are incorporated by reference herein in their entireties.

In certain embodiments when a staple is at the i and i+3 residues, R-propenylalanine and S-pentenylalanine; or R- pentenylalanine and S-pentenylalanine are substituted for the amino acids at those positions. In certain embodiments when a staple is at the i and i+4 residues, S-pentenyl alanine is substituted for the amino acids at those positions. In certain embodiments when a staple is at the i and i+7 residues, S-pentenyl alanine and R-octenyl alanine are substituted for the amino acids at those positions. In some instances, when the peptide is stitched, the amino acids of the peptide to be involved in the “stitch” are substituted with Bis-pentenylglycine, S-pentenylalanine, and R-octenylalanine; or Bis-pentenylglycine, S- octenylalanine, and R-octenylalanine.

Staple or stitch positions can be varied by testing different staple locations in a staple walk.

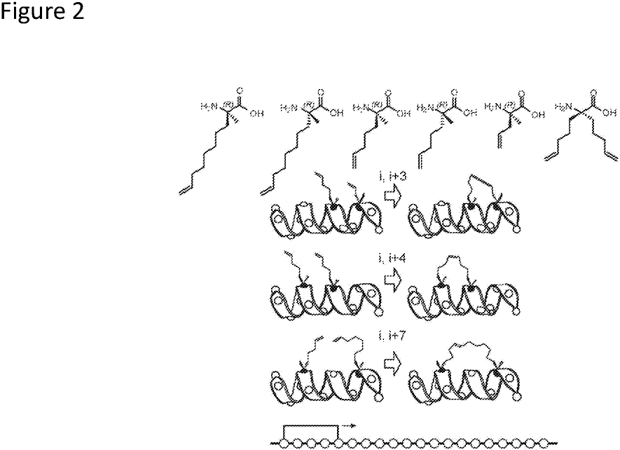

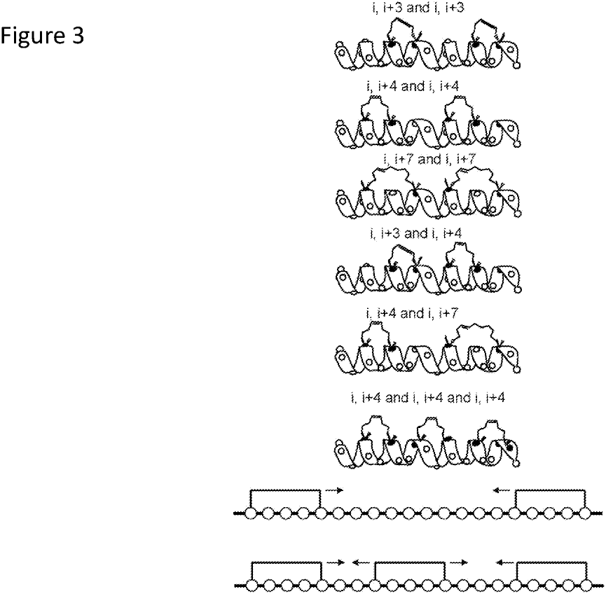

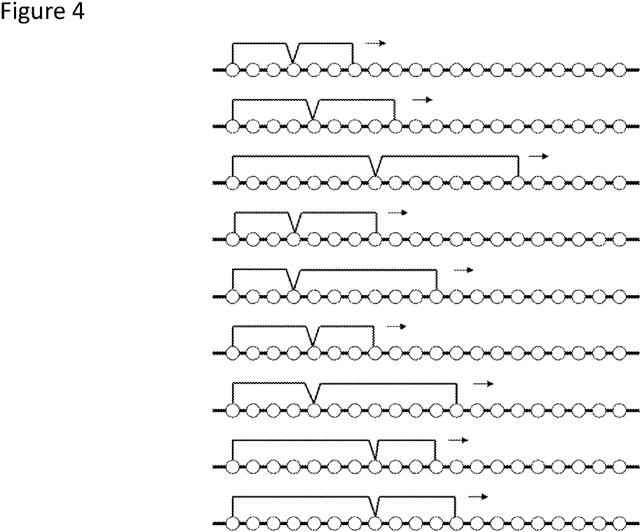

FIG. 2 (top) shows exemplary chemical structures of non-natural amino acids that can be used to generate various crosslinked compounds. FIG. 2 (middle) illustrates peptides with hydrocarbon cross-links between positions i and i+3; i and i+4; and i and i+7 residues. Fi. 2 (bottom) illustrates a staple walk along a peptide sequence. FIG. 3 shows various peptide sequences with double and triple stapling strategies, and exemplary staple walks. FIG. 4 illustrates exemplary staple walks using various lengths of branched stitched moieties.

In one aspect, a stabilized polypeptide has the formula (I),

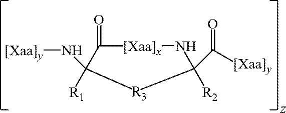

wherein:
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄-K-R₄]ₙ; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

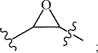

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);
and each Xaa is independently an amino acid.

The tether can include an alkyl, alkenyl, or alkynyl moiety (e.g., C₅, C₈, or C₁₁ alkyl, a C₅, C₈, or C₁₁ alkenyl, or C₅, C₈, or C₁₁ alkynyl). The tethered amino acid can be alpha disubstituted (e.g., C₁-C₃ or methyl).

In some instances, x is 2, 3, or 6. In some instances, each y is independently an integer between 1 and 15, or 3 and 15. In some instances, R₁ and R₂ are each independently H or C₁-C₆ alkyl. In some instances, R₁ and R₂ are each independently C₁-C₃ alkyl. In some instances, at least one of R₁ and R₂ are methyl. For example, R₁ and R₂ can both be methyl. In some instances, R₃ is alkyl (e.g., C₈ alkyl) and x is 3. In some instances, R₃ is C₁₁ alkyl and x is 6. In some instances, R₃ is alkenyl (e.g., C₈ alkenyl) and x is 3. In some instances, x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂—.

In another aspect, the two alpha, alpha disubstituted stereocenters are both in the R configuration or S configuration (e.g., i, i+4 cross-link), or one stereocenter is R and the other is S (e.g., i, i+7 cross-link). Thus, where formula I is depicted as:

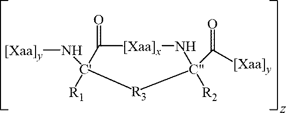

the C′ and C″ disubstituted stereocenters can both be in the R configuration or they can both be in the S configuration, e.g., when x is 3. When x is 6, the C′ disubstituted stereocenter is in the R configuration and the C″ disubstituted stereocenter is in the S configuration. The R₃ double bond can be in the E or Z stereochemical configuration.

In some instances, R₃ is [R₄-K-R₄]ₙ; and R₄ is a straight chain alkyl, alkenyl, or alkynyl.

In some embodiments, the disclosure features internally cross-linked (“stapled” or “stitched”) peptides, wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch. The stapled/stitched peptide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain instances, the stabilized peptide is a peptide of an intracellular protein that binds to a protein targeted for degradation (e.g., a coronaviral protease), a peptide that binds to E3 ubiquitin ligase or a peptide that binds to a substrate adaptor for an E3 ubiquitin ligase (e.g., a WD40-repeat protein).

Non-limiting examples of stapled peptides that can be incorporated into the chimeras of this disclosure are listed below:

ATVNVLAWLYAAVINGD (SEQ ID NO: 2)

-Mpro binding peptide

ATVNVLAWLYX₁ AVIX₂ GD (SEQ ID NO: 3)

- Mpro binding peptide

ANLNAGBX₁ LGSX₂ AATVELQ (SEQ ID NO: 4)

- NSP9 binding peptide

ANLNRGBX₁ LGSX₂ AATVRLQ (SEQ ID NO: 5)

- NSP9 binding peptide wherein, B = norleucine; and, X₁ and X₂ are the same (e.g., (S5)).

LTF(R8)EYWAQ#(S5)SAA (SEQ ID NO: 7)

- ATSP-7041, wherein # = cyclobutylalanine

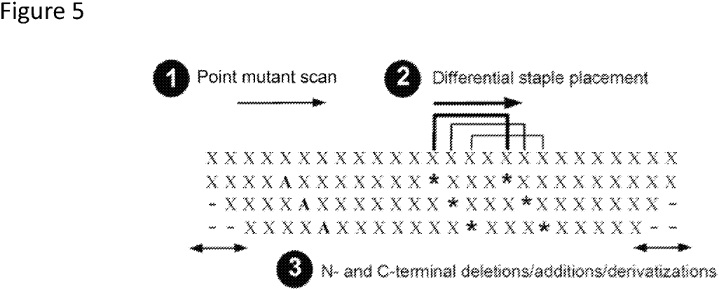

LTF(R8)EYWAQL(S5)SAA (SEQ ID NO: 1) - p53 (SP645)In certain embodiments, the stapled peptide comprises or consists of the amino acid sequence set forth in any one of SEQ ID NOs.: 1- 7, 12, 13, 38-47, and 49-50. In certain embodiments, this disclosure features stabilized peptides that differ from the peptides disclosed above in that they vary in the location of the staple/stitch. In certain embodiments, this disclosure features stabilized peptides that differ from the peptides disclosed above in that they vary from the above-disclosed sequences in having 1 to 7 (e.g., 1, 2, 3, 4, 5, 6, 7) amino acid substitutions on the non-interacting face of the alpha-helix of these peptides. In certain instances, the substitutions are conservative. In other instances, the substitutions are non-conservative. In certain embodiments, this disclosure features stabilized peptides that differ from the peptides disclosed above in that they vary from the above-disclosed sequences in having 1 to 5 (e.g., 1, 2, 3, 4, 5) amino acid substitutions on the interacting face of the alpha-helix of these peptides. In certain instances, the substitutions are conservative. Exemplary types of variations/modifications to stapled peptides are illustrated in FIG. 5.

### VII. Tethers in Stabilized Peptides

Hydrocarbon tethers are used in the stabilized peptides of this disclosure. In some instances, other tethers can also be employed in the stabilized peptides of this disclosure. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc.,113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U S. A., 97:3803-3808 (2000); Double-click stapling: Lau et al., Chem. Sci., 5:1804-1809 (2014); Bis-lactam stapling: J. C. Phelan et al.,, J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to i+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

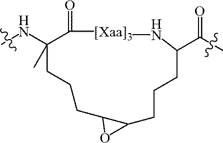

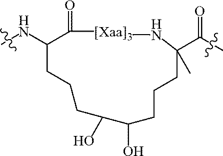

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the polypeptide or via the amino acid side chain. Other agents can be attached to the functionalized tether, e.g., an agent that facilitates entry of the polypeptide into cells.

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned.

The stapled polypeptides can include a drug, a toxin, a derivative of polyethylene glycol; a second polypeptide; a carbohydrate, etc. Where a polymer or other agent is linked to the stapled polypeptide it can be desirable for the composition to be substantially homogeneous.

The addition of polyethelene glycol (PEG) molecules can improve the pharmacokinetic and pharmacodynamic properties of the polypeptide. For example, PEGylation can reduce renal clearance and can result in a more stable plasma concentration. PEG is a water soluble polymer and can be represented as linked to the polypeptide as formula:

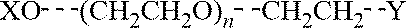

where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. 6,348,558.

In certain embodiments, macromolecular polymer (e.g., PEG) is attached to an agent described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n = 2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The stabilized peptides can also be modified, e.g., to further facilitate cellular uptake or increase in vivo stability, in some embodiments. For example, acylating or PEGylating a peptidomimetic macrocycle facilitates cellular uptake, increases bioavailability, increases blood circulation, alters pharmacokinetics, decreases immunogenicity and/or decreases the needed frequency of administration.

In some embodiments, the stapled peptides disclosed herein have an enhanced ability to penetrate cell membranes (e.g., relative to non-stapled peptides).

Methods of synthesizing the stabilized peptides described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T.W. Greene and P.G.M. Wuts, Protective Groups in Organic Synthesis, 3d. Ed., John Wiley and Sons (1999); L. Fieser and M. Fieser, Fieser and Fieser’s Reagents for Organic Synthesis, John Wiley and Sons (1994); and L. Paquette, ed., Encyclopedia of Reagents for Organic Synthesis, John Wiley and Sons (1995), and subsequent editions thereof.

The stabilized peptides can be made by chemical synthesis methods, which are well known to the ordinarily skilled artisan. See, for example, Fields et al., Chapter 3 in Synthetic Peptides: A User’s Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech. Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)-CHR) or CHR-C(O) wherein R is H or CH₃; and a fluoro-ketomethylene bond (C(O)-CFR or CFR-C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C1-C20 straight or branched alkyl groups); fatty acid radicals; and combinations thereof. α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). For peptides where an i linked to i+7 staple is used (two turns of the helix stabilized) either: a) one S5 amino acid and one R8 is used; or b) one S8 amino acid and one R5 amino acid is used. R8 is synthesized using the same route, except that the starting chiral auxillary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids Fmoc-S₅-OH, Fmoc-R₈-OH, Fmoc-R₈-OH, Fmoc-S₈-OH and Fmoc-R₅-OH), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stapling, and purifying the peptides disclosed herein that can be incorporated into the chimeras of this disclosure are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. Pat. Application No. 12/525,123, filed Mar. 18, 2010; and U.S. Pat. No. 7,723,468, issued May 25, 2010, each of which are hereby incorporated by reference in their entirety).

In some embodiments, the peptides are substantially free of non-stapled peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific embodiment, a 50%/50% DMSO/NMP solution is used. The solution may be incubated for a period of 1, 6, 12 or 24 hours, following which the resin may be washed, for example with dichloromethane or NMP. In one embodiment, the resin is washed with NMP. Shaking and bubbling an inert gas into the solution may be performed.

Properties of the stabilized (e.g., stapled) polypeptides of the disclosure can be assayed, for example, using the methods described in e.g., in WO 2019/118893, which is hereby incorporated by reference in its entirety.

### VIII. Linkers

There is no particular limitation on the linkers that can be used in between the first and second moieties of the chimeric constructs described above. In some embodiments, the linker is an amino acid such as amino-propionic-acid, aminobutanoic-acid, amino-pentanoic-acid, or amino-hexanoic-acid. In some embodiments, the linker is an oligoethylene glycol, i.e., NH₂—(CH₂—CH₂—O)ₓ—CH₂—CH₂—COOH. In some embodiments, the linker is a peptide linker. In some embodiments, any arbitrary single-chain peptide comprising about one to 30 residues (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids) can be used as a linker. In other embodiments, the linker is 10 to 20, 10 to 30, 10 to 40, 10 to 50, 10 to 60, 10 to 70, 10 to 80, 10 to 90, 10 to 100, 10 to 144, or 10 to 150 amino acids in length. In certain instances, the linker contains only glycine and/or serine residues. Examples of such peptide linkers include: Gly, Ser; Gly Ser; Gly Gly Ser; Ser Gly Gly; Gly Gly Gly Ser (SEQ ID NO: 16); Ser Gly Gly Gly (SEQ ID NO: 17); Gly Gly Gly Gly Ser (SEQ ID NO: 18); Ser Gly Gly Gly Gly (SEQ ID NO: 19); Gly Gly Gly Gly Gly Ser (SEQ ID NO: 20); Ser Gly Gly Gly Gly Gly (SEQ ID NO: 21); Gly Gly Gly Gly Gly Gly Ser (SEQ ID NO: 22); Ser Gly Gly Gly Gly Gly Gly (SEQ ID NO: 23); (Gly Gly Gly Gly Ser)n (SEQ ID NO: 24)n, wherein n is an integer of one or more; and (Ser Gly Gly Gly Gly)ₙ (SEQ ID NO: 25)n, wherein n is an integer of one or more. In some instances, the linker has multiple copies (e.g., 1, 2, 3, 4, 5, 6, 7, 8, 9, 10) of the amino acid sequence of SEQ ID NO: 16 with the exception that the serine residue in each copy of the linker is replaced with another amino acid.

In some embodiments, the linker is a peptide linker, a chemical linker, a Glycine-Serine linker, (G4S)₃ (SEQ ID NO: 26), (G4S)₅ (SEQ ID NO: 27), a beta-alanine (Z) linker, a beta-alanine and alanine (ZA) linker, or a polyethylene glycol linker. In some embodiments, the linker comprises beta-alanine. In another embodiment, the linker comprises a beta-alanine and alanine linker.

In other embodiments, the linker peptides are modified such that the amino acid sequence GSG (that occurs at the junction of traditional Gly/Ser linker peptide repeats) is not present. For example, the peptide linker comprise an amino acid sequence selected from the group consisting of: (GGGXX)ₙGGGGS (SEQ ID NO: 28) and GGGGS(XGGGS)ₙ (SEQ ID NO: 29), where X is any amino acid that can be inserted into the sequence and not result in a polypeptide comprising the sequence GSG, and n is 0 to 4. In one embodiment, the sequence of a linker peptide is (GGGX₁X₂)ₙGGGGS and X₁ is P and X₂ is S and n is 0 to 4 (SEQ ID NO: 30). In another embodiment, the sequence of a linker peptide is (GGGX₁X₂)ₙGGGGS and X₁ is G and X₂ is Q and n is 0 to 4 (SEQ ID NO: 31). In another embodiment, the sequence of a linker peptide is (GGGX₁X₂)ₙGGGGS and X₁ is G and X₂ is A and n is 0 to 4 (SEQ ID NO: 32). In yet another embodiment, the sequence of a linker peptide is GGGGS(XGGGS)ₙ, and X is P and n is 0 to 4 (SEQ ID NO: 33). In one embodiment, a linker peptide of the invention comprises or consists of the amino acid sequence (GGGGA)₂GGGGS (SEQ ID NO: 34). In another embodiment, a linker peptide comprises or consists of the amino acid sequence (GGGGQ)₂GGGGS (SEQ ID NO: 35). In yet another embodiment, a linker peptide comprises or consists of the amino acid sequence (GGGPS)₂GGGGS (SEQ ID NO: 36). In a further embodiment, a linker peptide comprises or consists of the amino acid sequence GGGGS(PGGGS)₂ (SEQ ID NO: 37).

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In certain embodiments, the linker is a linker depicted in FIG. 7.

### IX. Mpro Inhibitor Peptides

This disclosure features novel peptides having the sequence set forth in SEQ ID NO: 2 or 3, which peptides can bind and inhibit Mpro (e.g., inhibit Mpro dimerization and/or Mpro enzymatic activity), thereby inhibiting viral (e.g., coronaviral) maturation and/or replication. This disclosure also features variants, e.g., sequences that differ by 1, 2, 3, 4, 5, 6, 7, 8, 9, 10, 11, 12, or 13 amino acids from SEQ ID NO: 2 or 3, wherein the variants inhibit Mpro. In some instances, the peptides can have at least or about 30%, at least or about 40%, at least or about 50%, at least or about 60%, at least or about 70%, at least or about 75% identity, at least or about 80%, at least 85%, at least or about 90%, at least or about 95%, at least or about 98%, at least or about 99%, or 100% identity to those amino acids in SEQ ID NO: 2 or 3, wherein the peptides bind to Mpro and inhibit Mpro dimerization and/or Mpro enzymatic activity. In some instances, these peptides inhibit coronaviral (e.g., SARS-CoV2) activity (e.g., inhibit viral maturation and replication).

In some embodiments, an Mpro inhibitor peptide is a recombinant or synthetically produced peptide. Such peptides can be non-cross-linked, stapled, or stitched, so long as the peptides interact with Mpro as described herein and inhibit its dimerization and/or enzymatic activity. In certain embodiments, the peptides differ from the peptides of SEQ ID NO: 2 or 3 in that they vary from SEQ ID NO: 2 or 3 in having 1 to 5 (e.g., 1, 2, 3, 4, 5) amino acid substitutions. In some cases a non-natural amino acid (e.g., S5) with olefinic side chains is inserted at positions 3 and 7 of SEQ ID NO: 2. In some cases a non-natural amino acid with olefinic side chains (e.g., S5) is inserted at positions 11 and 15 of SEQ ID NO: 2. In certain cases, a non-natural amino acid (e.g., S5) with olefinic side chains is inserted at positions 3 and 7 and positions 11 and 15 of SEQ ID NO: 2. Each of these stapled peptides can further include 1, 2, 3, 4, or 5 amino acid substitutions so long as these stapled peptides retain their ability to bind and inhibit Mpro. For instance, the positions labeled “X” can be substituted in SEQ ID NO: 2 or 3 as follows: ATXNVLXWLYXAVIXGD (SEQ ID NO: 51). X can be a conservative or non-conservative amino acid residue. In some embodiments, “X” is a non-natural amino acid such as (S)-2-(4′-pentenyl)alanine (S5). In those embodiments, the peptide can be single stapled or double stapled between the positions i and i+4.

The enzymatic activity of Mpro relies on forming a homodimer mediated by an alpha-helical sequence: TVNVLAWLYAAVINGD (SEQ ID NO: 9). SEQ ID NO: 9 can be used to create peptide-based dimerization inhibitors. In some instances, Mpro inhibitor peptides can include at least six (e.g., 7, 8, 9, 10, 11, 12, 13, 14, 15, or 16 contiguous amino acids of SEQ ID NO: 9). In some instances, the Mpro binder peptide can be a variant of SEQ ID NO: 9 - e.g., differing from SEQ ID NO: 9 by 1, 2, 3, 4, 5, or 6 amino acid substitutions, deletions, and/or insertions, wherein the variant can still dimerize with Mpro.

In some embodiments, Mpro inhibitor peptides can comprise, consist, or consist essentially of the amino acid sequences of e.g., SEQ ID NO: 2 or 3. In some embodiments, the peptides can comprise, consist, or consist essentially of amino acid sequences related or with identity to a portion or portions of the amino acid sequence of e.g., SEQ ID NO: 2 or3.

Alternatively or in addition, the peptides can include amino acid substitutions and/or deletions, whether conservative or not. For example, the peptide can include 0, 1, 2, 3, 4, 5, 6, 7, 8, 9, less than 10, less than 5, less than 4, less than 3, or less than 2 amino acid substitutions, deletions, and/or additions, whether conservative or not, provided that the peptide can still bind to Mpro and inhibit its dimerization and/or enzymatic activity. Accordingly, the amino acid sequence of any of the Mpro inhibitor peptides disclosed herein can be varied so long as the variant peptide binds to and inhibits Mpro.

### X. NSP9 Inhibitor Peptides

This disclosure features novel peptides having the sequence set forth in SEQ ID NO: 4 or 5, which peptides can bind and inhibit NSP9 (e.g., inhibit NSP9 dimerization and/or NSP9 enzymatic activity), thereby inhibiting viral (e.g., coronaviral) maturation and/or replication. In some embodiments, an NSP9 inhibitor peptide is a recombinant or synthetically produced peptide. Such peptides can be non-cross-linked, stapled, or stitched, so long as the peptides interact with NSP9 as described herein.

The enzymatic activity of NSP9 relies on forming a homodimer mediated by an alpha-helical sequence: NLNRGMVLGSLAATVRLQ (SEQ ID NO: 10). SEQ ID NO: 10 can be used to create peptide-based dimerization binders. In some instances, NSP9 inhibitor peptides can include at least six (e.g., 7, 8, 9, 10, 11, 12, 13, 14, 15, 16, 17, or 18 contiguous amino acids of SEQ ID NO: 10). In some instances, NSP9 inhibitor peptides can include at least 1, 2, 3, 4, 5, 6, 7, or 8 amino acid substitutions or deletions in SEQ ID NO: 10 so long as the variant peptide still binds and inhibits NSP9. In some instances, an NSP9 inhibitor peptide includes the sequence GXXXG (SEQ ID NO: 8).

In some embodiments, NSP9 inhibitor peptides can comprise, consist, or consist essentially of the amino acid sequences of e.g., SEQ ID NO: 4 or 5. In some embodiments, the peptides can include amino acid sequences related or with identity to a portion or portions of the amino acid sequence of e.g., SEQ ID NO: 4 or 5.

In some instances, the peptides can have at least or about 30%, at least or about 40%, at least or about 50%, at least or about 60%, at least or about 70%, at least or about 80%, at least 85%, at least or about 90%, at least or about 95%, at least or about 98%, at least or about 99%, or 100% identity to those amino acids in SEQ ID NO: 4 or 5, wherein the peptides bind to NSP9. Alternatively or in addition, the peptides can include amino acid substitutions and/or deletions, whether conservative or not. For example, amino acids can include 0, 1, 2, 3, 4, 5, 6, 7, 8, less than 10, less than 5, less than 4, less than 3, or less than 2 amino acid substitutions, deletions, and/or additions, whether conservative or not. Accordingly, the amino acid sequence of any NSP9 inhibitor peptide disclosed herein can be varied so long as the variant peptide can still binds to and inhibits NSP9.

In some instances, an NSP9 inhibitor peptide of SEQ ID NO: 4 or 5 or variants thereof can be shortened by 1, 2, or 3 amino acids at each end of the sequence. In other instances, an NSP9 inhibitor peptide of SEQ ID NO: 4 or 5 or variants thereof can include either no staple, one staple (e.g., a staple formed between S5 and S5 (i, i+4 positions), R8 and S5 (i, i+7 positions), or S5 and R8 (i, i+7)), or be double stapled. In certain embodiments, the peptides differ from the peptides of SEQ ID NO: 4 or 5 in that they vary from SEQ ID NO: 4 or 5 in having 1 to 4 (e.g., 1, 2, 3, 4) amino acid substitutions. For instance, the positions labeled “X” can be substituted in SEQ ID NO: 4 as follows: ANLNAGBXLGSXAATVELQ (SEQ ID NO: 52). X can be a conservative or non-conservative amino acid residue. In some embodiments, “X” is a non-natural amino acid such as (S)-2-(4′-pentenyl)alanine (S5). In those embodiments, the peptide can be single stapled between the positions i and i+4. In some instances, the core peptide sequence that is required is the

GX₁X₂X₃G motif (SEQ ID NO: 8), wherein X₁, X₂, and X₃ can be any amino acid. In some cases, X₁ = M, norleucine (B), A, or G; X₂ = V, A, G, M or B; and X₃ = L, A, G, V, or I.

### XI. Methods of Treatment

The chimeras disclosed herein can facilitate degradation of the target protein (e.g., a viral protein such as a coronaviral protease, a coronaviral NSP; or a host BET protein) to which the chimeric molecule binds. In certain instances, the protein that is degraded by a chimera of this disclosure is a coronaviral PLpro protein. In some instances, a coronaviral Mpro protein is degraded by a chimera of this disclosure. In other instances, a coronaviral NSP9 protein is degraded by a chimera of this disclosure. In certain instances, a coronaviral Mpro protein is degraded by a chimera of this disclosure. In yet other instances, a host BRD2 protein is degraded by a chimera of this disclosure. In some instances, a host BRD3 protein is degraded by a chimera of this disclosure. In yet some instances, a host BRD4 protein is degraded by a chimera of this disclosure. Degradation of the coronaviral protease, coronaviral NSP, and/or host BET protein is useful for e.g., in treating a coronaviral infection, inhibiting coronaviral replication, reducing coronaviral load, treating symptoms associated with a coronaviral infection or disease (such as COVID-19), reducing viral pathogenicity, reducing the time to coronaviral clearance, and reducing mortality in the clinical outcomes in subjects with coronaviral infections. The chimeras of this disclosure further provide methods of reducing the risk that the individual will develop coronaviral disease (such as SARS or COVID-19).). In some instances, treating a coronaviral infection with the chimera of this disclosure reduces symptoms such as fever or chills, cough, shortness of breath or difficulty breathing, fatigue, muscle or body aches, headache, loss of taste or smell, sore throat, congestion or runny nose, nausea or vomiting, and diarrhea.

The disclosure features methods of using any of the chimeras described herein for the prophylaxis and/or treatment of an infection or disease caused by an RNA virus (e.g., a coronavirus such as SARS-CoV2) in a subject in need thereof (e.g., a human subject). As used herein, the terms “treat” “treating,” and the like, refer to alleviating, inhibiting, or ameliorating the disease or condition from which the subject is suffering. For example, a chimera of this disclosure can be used to treat COVID-19 caused by SARS-CoV2. The disclosure also features methods of completely or partially preventing a disease (e.g., COVID-19) or symptom thereof.

The present disclosure further provides methods of therapeutically treating a coronavirus (e.g., SARS-CoV-2) infection in a subject (e.g., a human, a primate, a bat, a bird, a mouse, a turkey, a cow, a pig, a cat, a dog, etc) who presents with clinical signs of coronavirus infection following known or suspected exposure to coronavirus. A subject who has been in close contact with an individual who has been diagnosed with a coronavirus infection, and who presents with one or more symptoms including but not limited to fever (often exceeding 38° C.), chills, cough, shortness of breath or difficulty breathing, fatigue, muscle or body aches, headache, loss of taste, loss of smell, sore throat, congestion or runny nose, nausea or vomiting, diarrhea are considered eligible for treatment with the methods of the disclosure. An advantage of the subject methods is that the severity of the coronavirus infection is reduced, e.g., the viral load is reduced, and/or the time to viral clearance is reduced, and/or the morbidity or mortality is reduced.

In some embodiments, the disclosure provides a method for treating or preventing a coronaviral (e.g., SARS-CoV-2) infection in a subject in need or at risk thereof, by administering to the subject a composition comprising a chimeric composition of the disclosure in combination with one or more antiviral or other agents including, but not limited to corticosteroid, hydrocortisone, methylprednisolone, dexamethasone, remdesivir, an IL-6 inhibitor, an IL-1 inhibitor, a kinase inhibitor, a complement inhibitor, ivermectin, hydroxychloroquine, favipiravir, and interferon-beta.

The present disclosure provides methods of prophylactically treating a coronavirus infection in an individual who is not yet infected with a coronavirus and/or who does not exhibit symptoms typical of a coronavirus infection. Such individuals include those who have been in contact with an individual who has COVID-19, including, e.g., health care personnel; persons in confined spaces with an individual who has COVID-19 (e.g., commercial airline workers such as flight attendants and pilots; travelers; conference attendees; and the like); and persons living in the same household with an individual who has COVID-19. An advantage of the present disclosure is that the risk that the individual will develop a pathological coronavirus infection is reduced.

As used herein, the term “coronavirus” includes any member of the family Coronaviridae, including, but not limited to, any member of the genus Coronavirus. The genus Coronavirus includes but is not limited to Alphacoronavirus strains (such as human coronavirus 229E (HCoV-229E), and HCoV-NL63), Betacoronavirus strains (such as HCoV-OC43, HCoV Hong Kong University 1 (HCoV-HKU1), Middle East Respiratory Syndrome coronavirus (MERS-CoV), severe acute respiratory syndrome coronavirus (SARS-CoV), and SARS-CoV-2), Gammacoronavirus (such as Avian infectious bronchitis virus, and turkey coronavirus) and Deltacoronavirus strains (such as porcine coronavirus HKU15, white-eye coronavirus HKU16, sparrow coronavirus HKU17, magpie robin coronavirus HKU18, night heron coronavirus HKU19, wigeon coronavirus HKU20, and common moorhen coronavirus HKU21). In some embodiments, a subject is infected with SARS-CoV-2 and can be treated with a chimera of this disclosure. The term “coronavirus’ further includes naturally-occurring (e.g., wild-type) coronavirus; naturally-occurring coronavirus variants; and coronavirus variants generated in the laboratory, including variants generated by selection, variants generated by chemical modification, and genetically modified variants (e.g., coronavirus modified in a laboratory by recombinant DNA methods).

In general, methods include selecting a subject and administering to the subject an effective amount of one or more of the chimeras of the disclosure, e.g., in or as a pharmaceutical composition, and optionally repeating administration as required for the prophylaxis or treatment of a coronaviral infection, e.g., SARS or COVID-19, and can be administered orally, intravenously, topically, buccally, rectally, parenterally, intraperitoneally, intradermally, subcutaneously, intramuscularly, transdermally, intranasally, pulmonarily, or intratracheally. A subject can be selected for treatment based on, e.g., determining that the subject has or is suspected of having a coronaviral infection.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient’s disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

In some embodiments, the chimera of the disclosure and the method of treatment is used prophylactically. For example, the method comprises administering to a subject determined to be susceptible to or at risk of acquiring a coronaviral infection, the chimera, and/or the composition comprising a chimera described herein.

In some embodiments, the chimera described herein may be administered in combination with one or more known and suitable medicaments for treating viral infections, including one or more agents selected from a list comprising corticosteroid, hydrocortisone, methylprednisolone, dexamethasone, remdesivir, an IL-6 inhibitor, an IL-1 inhibitor, a kinase inhibitor, a complement inhibitor, ivermectin, hydroxy chloroquine, favipiravir, interferon-beta, and icatibant. In that context, the one or more agents and the chimera of the disclosure may be administered simultaneously, or sequentially with the composition comprising the chimera, i.e., in a single formulation or in separate formulations packaged either together or individually.

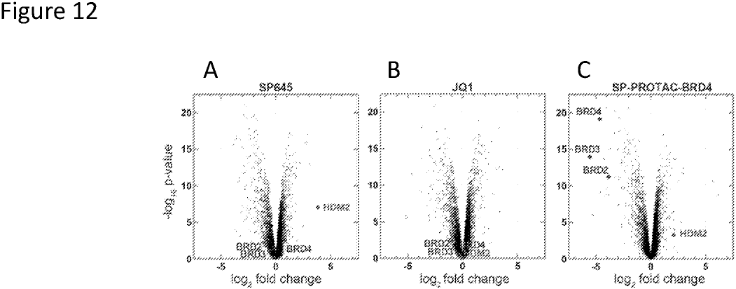

In some embodiments, the chimera of this disclosure can have a dual effect by synergistically degrading a target protein necessary for viral replication and/or pathogenesis (such as PLpro protein or BRD) and increasing p53 levels in host cells as shown in FIG. 1 and FIGS. 12A-C. See also, Example 4. Further, because PLpro is also involved in viral mechanisms of evading the host immune system, a chimera of this disclosure can both block viral replication and restore the capacity of the immune system to kill infected cells.

### XII. Pharmaceutical Compositions

One or more of any of the chimeras described herein can be formulated for use as or in pharmaceutical compositions. Such compositions can be formulated or adapted for administration to a subject via any route, e.g., any route approved by the Food and Drug Administration (FDA). Exemplary methods are described in the FDA’s CDER Data Standards Manual, version number 004 (which is available at fda.give/cder/dsm/DRG/drg00301.htm). For example, compositions can be formulated or adapted for administration by oral, intravenous, topical, buccal, rectal, parenteral, intraperitoneal, intradermal, subcutaneous, intramuscular, transdermal, intranasal, pulmonary, or intratracheal routes. These routes include inhalation (e.g., oral and/or nasal inhalation (e.g., via nebulizer or spray)), injection (e.g., intravenously, intra-arterially, subdermally, intraperitoneally, intramuscularly, and/or subcutaneously); and/or for oral administration, transmucosal administration, and/or topical administration (including topical (e.g., nasal) sprays and/or solutions). The compositions of this disclosure may be administered with a dosage ranging from about 0.001 to about 100 mg/kg of body weight, or according to the requirements of the particular drug. Alternatively, or in addition, the compositions may be administered according to any of the Food and Drug Administration approved methods, for example, as described in the FDA Data Standards Manual (DSM) (available at www.fda.gov/Drugs/DevelopmentApprovalProcess/FormsSubmissionRequirements/E lectronicSubmissions/DataStandardsManualmonographs).

Typically, the pharmaceutical compositions of this disclosure will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active chimera (w/w). Alternatively, such preparations contain from about 20% to about 80% active chimera.

In some embodiments, an effective dose of a chimera of this disclosure can include, but is not limited to, e.g., about, 0.00001, 0.0001, 0.001, 0.01, 0.1, 1 or 10-10000; 0.00001, 0.0001, 0.001, 0.01, 0.1, 1 or 10-5000; 0.00001, 0.0001, 0.001, 0.01, 0.1, 1 or 10-2500; 0.00001, 0.0001, 0.001, 0.01, 0.1, 1 or 10-1000; 0.00001, 0.0001, 0.001, 0.01, 0.1, 1 or 10-900; 0.00001, 0.0001, 0.001, 0.01, 0.1, 1 or 10-800; 0.00001, 0.0001, 0.001, 0.01, 0.1, 1 or 10-700; 0.00001, 0.0001, 0.001, 0.01, 0.1, 1 or 10-600; 0.00001, 0.0001, 0.001, 0.01, 0.1, 1 or 10-500; 0.00001, 0.0001, 0.001, 0.01, 0.1, 1 or 10-400; 0.00001, 0.0001, 0.001, 0.01, 0.1, 1 or 10-300; 0.00001, 0.0001, 0.001, 0.01, 0.1, 1 or 10-200; 0.00001, 0.0001, 0.001, 0.01, 0.1, 1 or 10-100; 0.00001, 0.0001, 0.001, 0.01, 0.1, 1 or 10-90; 0.00001, 0.0001, 0.001, 0.01, 0.1, 1 or 10-80; 0.00001, 0.0001, 0.001, 0.01, 0.1, 1 or 10-70; 0.00001, 0.0001, 0.001, 0.01, 0.1, 1 or 10-60; 0.00001, 0.0001, 0.001, 0.01, 0.1, 1 or 10-50; 0.00001, 0.0001, 0.001, 0.01, 0.1, 1 or 10-40; 0.00001, 0.0001, 0.001, 0.01, 0.1, 1 or 10-30; 0.00001, 0.0001, 0.001, 0.01, 0.1, 1 or 10-20; 0.00001, 0.0001, 0.001, 0.01, 0.1, 1 or 10-30; 0.00001, 0.0001, 0.001, 0.01, 0.1, 1-15, 0.00001, 0.0001, 0.001, 0.01, 0.1, 1 or 10-30; 0.00001, 0.0001, 0.001, 0.01, 0.1, 1-10, 0.00001, 0.0001, 0.001, 0.01, 0.1, 1 or 10-30; or 0.00001, 0.0001, 0.001, 0.01, 0.1, 1-5 mg/kg/day, e.g., administered intravenously.

Lower or higher doses than those recited above may be required. Specific dosage and treatment regimens for any particular subject will depend upon a variety of factors, including the activity of the specific chimera employed, the age, body weight, general health status, sex, diet, time of administration, rate of excretion, drug combination, the severity and course of the disease, condition or symptoms, the subject’s disposition to the disease, condition or symptoms, and the judgment of the treating physician.

Upon improvement of a patient’s condition, a maintenance dose of a chimera, composition or combination of this disclosure may be administered, if necessary. Subsequently, the dosage or frequency of administration, or both, may be reduced, as a function of the symptoms, to a level at which the improved condition is retained. Subjects may, however, require intermittent treatment on a long-term basis upon any recurrence of disease symptoms.

The chimeras in the compositions of this disclosure may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In some instances, pharmaceutical compositions can include an effective amount of one or more chimeras. The terms “effective amount” and “effective to treat,” as used herein, refer to an amount or a concentration of one or more chimeras or a pharmaceutical composition described herein utilized for a period of time (including acute or chronic administration and periodic or continuous administration) that is effective within the context of its administration for causing an intended effect or physiological outcome (e.g., treatment of coronaviral infection). Effective amounts of one or more chimeras or a pharmaceutical composition described herein include amounts that promote decreased levels of the target protein (e.g., coronaviral PLpro, coronaviral Mpro, coronaviral NSP9, coronaviral NSP12, host BRD2, host BRD3, or host BRD4) and/or increased host p53 levels (e.g., protein levels) and/or p53 activity (e.g., biological activity) in a cell. A therapeutically effective amount of a chimera is not required to cure a condition (e.g., COVID-19) but will provide a treatment for the condition.

Pharmaceutical compositions of this disclosure can include one or more chimeras and any pharmaceutically acceptable carrier and/or vehicle. In some instances, a pharmaceutical composition can further include one or more additional therapeutic agents in amounts effective for achieving a modulation or amelioration of disease or infection (e.g., COVID-19) or disease symptoms. The one or more therapeutic agents include but are not limited to a corticosteroid, hydrocortisone, methylprednisolone, dexamethasone, remdesivir, an IL-6 inhibitor, an IL-1 inhibitor, a kinase inhibitor, a complement inhibitor, ivermectin, hydroxychloroquine, favipiravir, interferon-beta, and icatibant.

The term “pharmaceutically acceptable carrier” refers to a carrier or adjuvant that may be administered to a subject, together with a chimera of this disclosure, and which does not destroy the pharmacological activity thereof and is nontoxic when administered in doses sufficient to deliver a therapeutic amount of the chimera.

Pharmaceutically acceptable carriers, adjuvants and vehicles that may be used in the pharmaceutical compositions of this disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of the chimeras described herein.

Pharmaceutically acceptable salts of the chimeras of this disclosure include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include alkali metal (e.g., sodium), alkaline earth metal (e.g., magnesium), ammonium and N-(alkyl)4+ salts.

The pharmaceutical compositions of this disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the chimera or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

Pharmaceutical compositions can be in the form of a solution or powder for inhalation and/or nasal administration. Such compositions may be formulated according to techniques known in the art using suitable dispersing or wetting agents (such as, for example, Tween 80) and suspending agents. For instance, such compositions may be prepared as solutions in saline, employing benzyl alcohol or other suitable preservatives, absorption promoters to enhance bioavailability, fluorocarbons, and/or other solubilizing or dispersing agents known in the art.

Pharmaceutical compositions can be in the form of sterile injectable preparation, such as a sterile injectable solution or suspension in a non-toxic parenterally acceptable diluent or solvent, for example, as a solution in 1,3-butanediol. Among the acceptable vehicles and solvents that may be employed are mannitol, water, Ringer’s solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Pharmaceutical compositions can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this disclosure may also be administered in the form of suppositories for rectal administration. These compositions can be prepared by mixing a chimera of this disclosure with a suitable non-irritating excipient which is solid at room temperature but liquid at the rectal temperature and therefore will melt in the rectum to release the active components. Such materials include, but are not limited to, e.g., cocoa butter, beeswax, and polyethylene glycols.

When the compositions of this disclosure comprise a combination of a chimera described herein and one or more additional therapeutic or prophylactic agents, both the chimera and the additional agent should be present at dosage levels of between about 1 to 100%, and more preferably between about 5 to 95% of the dosage normally administered in a monotherapy regimen. The additional agents may be administered separately, e.g., as part of a multiple dose regimen, from the chimera of this disclosure. Alternatively, those agents may be part of a single dosage form, mixed together with the chimera of this disclosure in a single composition.

In some instances, one or more chimeras disclosed herein can be conjugated, for example, to a carrier protein. Such conjugated compositions can be monovalent or multivalent. For example, conjugated compositions can include one peptide disclosed herein conjugated to a carrier protein. Alternatively, conjugated compositions can include two or more peptides disclosed herein conjugated to a carrier.

As used herein, when two entities are “conjugated” to one another they are linked by a direct or indirect covalent or non-covalent interaction. In certain embodiments, the association is covalent. In other embodiments, the association is non-covalent. Non- covalent interactions include hydrogen bonding, van der Waals interactions, hydrophobic interactions, magnetic interactions, electrostatic interactions, etc. An indirect covalent interaction is when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561,1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

In some embodiments, the present disclosure provides methods for using any one or more of the chimeras (indicated below as ‘X’) disclosed herein in the following methods:

Chimera X for use as a medicament in the treatment of one or more conditions disclosed herein (e.g., COVID-19, referred to in the following examples as ‘Y’). Use of chimera X for the manufacture of a medicament for the treatment of Y; and chimera X for use in the treatment of Y.

TABLE 4

Sequence Listings

| SEQ ID NO: | DESCRIPTION | SEQUENCE |
|---|---|---|
| 1 | HDM2 binding peptide SP645 | LTF(R8)EYWAQL(S5)SAA |
| 2 | Mpro binding peptide | ATVNVLAWLYAAVINGD |
| 3 | Mpro binding peptide | ATVNVLAWLYX₁AVIX₂GD |
| 4 | NSP9 binding peptide | ANLNAGBX₁LGSX₂AATVELQ |
| 5 | NSP9 binding peptide | ANLNRGBX₁LGSX₂AATVRLQ |
| 6 | Trib1 | DQIVPEY |
| 7 | ATSP-7041 | LTF(R8)EYWAQ#(S5)SAA |
| 8 | NSP9 motif | GXXXG |
| 9 | Mpro alpha helical sequence | TVNVLAWLYAAVINGD |
| 10 | NSP9 alpha helical sequence | NLNRGMVLGSLAATVRLQ |
| 11 | WT human p53 aa 14-29 | LSQETFSDLWKLLPEN |
| 12 | VHL binding peptide | LAPAAGDTIISLDF |
| 13 | VHL binding peptide | LAPYIPMDDDFQL |
| 14 | MDM2_SWIB motif pattern | F[^P] {3} W[^P] {2,3} [VIL] |
| 15 | ODPH_VHL_1 motif pattern | [IL]A(P). {6,8} [FLIVM].[FLIVM] |
| 16 | Linker sequence | GGGS |
| 17 | Linker sequence | SGGG |
| 18 | Linker sequence | GGGGS |
| 19 | Linker sequence | SGGGG |
| 20 | Linker sequence | GGGGGS |
| 21 | Linker sequence | SGGGGG |
| 22 | Linker sequence | GGGGGGS |
| 23 | Linker sequence | SGGGGGG |
| 24 | Linker sequence | (GGGGS)ₙ; wherein n=1 or more |
| 25 | Linker sequence | (SGGGG)ₙ; wherein n=1 or more |
| 26 | Linker sequence | GGGGSGGGGSGGGGS |
| 27 | Linker sequence | GGGGSGGGGSGGGGSGGGGSGGGGS |
| 28 | Linker sequence | (GGGXX)nGGGGS |
| 29 | Linker sequence | GGGGS(XGGGS)ₙ |
| 30 | Linker sequence | (GGGX₁X₂)ₙGGGGS; wherein X₁ is P and X₂ is S and n is 0 to 4 |
| 31 | Linker sequence | (GGGX₁X₂)ₙGGGGS; wherein X₁ is G and X₂ is Q and n is 0 to 4 |
| 32 | Linker sequence | (GGGX₁X₂)ₙGGGGS; wherein X₁ is G and X₂ is A and n is 0 to 4 |
| 33 | Linker sequence | GGGGS(XGGGS)n; wherein X is P and n is 0 to 4 |
| 34 | Linker sequence | (GGGGA)₂GGGGS |
| 35 | Linker sequence | (GGGGQ)₂GGGGS |
| 36 | Linker sequence | (GGGPS)₂GGGGS |
| 37 | Linker sequence | GGGGS(PGGGS)₂ |
| 38 | p53 transactivation domain sequence | FSDLWKLL |
| 39 | PMI duodecimal peptide inhibitor | TSFAEYWNLLSP |
| 40 | SAH-p53-1 | LSQETFSD(R8)WKLLPE(S5) |
| 41 | SAH-p53-2 | LSQE(R8)FSDLWK(S5)LPEN |
| 42 | SAH-p53-3 | LSQ(R8)TFSDLW(S5)LLPEN |
| 43 | SAH-p53-4 | LSQETF(R8)DLWKLL(S5)EN |
| 44 | SAH-p53-5 | LSQETF(R8)NLWKLL(S5)QN |
| 45 | SAH-p53-6 | LSQQTF(R8)NLWRLL(S5)QN |
| 46 | SAH-p53-7 | QSQQTF(R8)NLWKLL(S5)QN |
| 47 | SAH-p53-8 | QSQQTF(R8)NLWRLL(S5)QN |
| 48 | SP645 variant sequence 1 | X₁TF(R8)X₂YX₃AQX₄(S5)X₅AA |
| 49 | SP645 variant sequence 2 | LTF(R5)EYWAQL(S8)SAA |
| 50 | ATSP-7041 variant sequence | LTF(R5)EYWAQ#(S8)SAA |
| 51 | Mpro binding peptide variant sequence | ATXNVLXWLYXAVIXGD |
| 52 | NSP9 binding peptide variant sequence | ANLNAGBXLGSXAATVELQ |
| 53 | N-terminal hexahistidine tag | HHHHHH |
| 54 | NSP9-SP645 (SP-PROTAC-NSP9-1) | ANLNAGB(S5)LGS(S5)AATVELQZA LTF(R8)EYWAQL(S5)SAA |
| 55 | SP645-NSP9 (SP-PROTAC-NSP9-2) | LTF(R8)EYWAQL(S5)SAAZ ANLNAGB(S5)LGS(S5)AATVELQ |

The motif patterns in SEQ ID NOs.: 14 and 15 use the following nomenclature: ‘.’ specifies any amino acid type, ‘[X]’ specifies the allowed amino acid type(s) at that position, ‘^X’ at the beginning of the pattern specifies that the sequence starts with amino acid type X, ‘[^X]’ denotes that the position can have any amino acid other than type X, numbers specified as the following ‘X{x,y}’, where x and y specify the minimum and maximum number of ‘X’ amino acid type required at that position. Conserved residue positions within the primary degron that are known to be post-translationally modified (for example, phosphorylation and proline hydroxylation) are shown in boldface. In SEQ ID NOs.: 1, 7, 40-48, and 54-55, (R8) is (R)-2-(7′-octenyl)alanine, and (S5) is (S)-2-(4′-pentenyl)alanine. In SEQ ID NO:48, X₁, X₂, and X₅ are any amino acid (e.g., A, W, F, L, V, I, naphthylalanine, E, D, Y, cyclobutylalanine and side chain analogs thereof); X₃: W, F, or 3-(2-naphthyl)-L-alanine; and X₄: L or a leucine mimetic (e.g., cyclobutylalanine). In SEQ ID NOs.: 49-50, (R5) is (R)-2-(4′-pentenyl)alanine and (S8) is (S)-2-(7′-octenyl)alanine. In SEQ ID NOs.: 7 and 50, # is cyclobutylalanine. In SEQ ID NOs.: 3-5, X₁ and X₂ are (S)-2-(4′-pentenyl)alanine. In SEQ ID NOs.: 4-5, B is norleucine. In SEQ ID NOs.: 28-29, X is any amino acid that can be inserted into the sequence and not result in a polypeptide comprising the sequence GSG, and n is 0 to 4. In SEQ ID NOs.: 51-52, X is (S)-2-(4′-pentenyl)alanine. In SEQ ID NOs.: 54-55, Z is beta-alanine.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Synthesis of SP-PROTACs to Degrade Essential Proteins of the SARS-CoV-2 Life Cycle

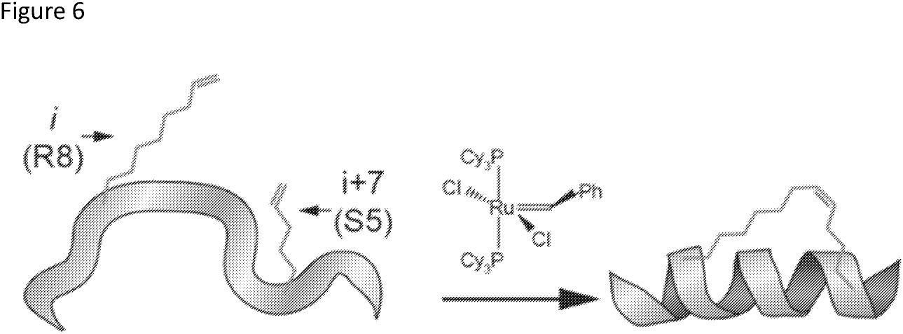

Stapled-peptide proteolysis-targeting chimeras (SP-PROTACs) were generated for targeted SARS-CoV-2 protein degradation by synthesizing the stapled or stitched p53 peptide on-resin, appending a linker of variable length (e.g., β-alanines, PEGs, Gly-Ser linker, etc.), followed by the selected small molecule, derivatized as needed for the condensation reaction. FIGS. 2-5 demonstrate the diversity of approaches in designing the stabilized (i.e., stapled or stitched) peptide portions of these chimeras. For example, SP645 was synthesized by replacing two amino acids with the non-natural amino acids S-octenyl alanine and R-pentenyl alanine flanking 6 amino acids (i, i+7 position) (see, FIG. 6). FIG. 7 shows the structure of SP645 (SEQ ID NO: 1). Syntheses of the requisite α,α-disubstituted amino acids was performed as described previously (Walensky, L.D. et al. Science 305, 1466-70 (2004); Schafmeister, C.E., et al. J. Am. Chem. Soc. 122, 5891-5892 (2000)). Solid-phase Fmoc chemistry and ruthenium-catalyzed olefin metathesis were used for peptide synthesis and staple formation, followed by appending the linker and small molecule on-resin using standard coupling chemistries.

To incorporate the PLpro inhibitor GRL-0617, for example, chemical substitution was achieved via its NH₂ group. GRL-0617-acid (11.3 mg, 0.0281 mmol, 1 eq) was combined with N-(4-aminobutyl)-2-((2-(2,6-dioxopiperidin-3-yl)-1,3-dioxoisoindolin-4-yl)oxy)acetamidetrifluoroacetate (14.5 mg, 0.0281 mmol, 1eq) dissolved in DMF (0.28 ml, 0.1 M) at room temperature. DIPEA (14.7 microliters, 0.0843 mmol, 3 eq) and HATU (10.7 mg, 0.0281 mmol, 1 eq) was then added to the peptide, the N-terminal Fmoc of which was previously deprotected with piperidine, and the mixture was reacted under nitrogen for 20 hours. The final SP-PROTAC was obtained upon peptide deprotection and cleavage, purification by LC/MS, and quantitation by amino acid analysis. C-termini of SP-PROTACs were further derivatized with FITC for binding analyses.

The prototype panel of SP-PROTACs incorporated PLpro and Mpro targeting small molecules, including reported compounds as described in Baez-Santos, et al. J. Virol., 88, 12511-27 (2014); Ghosh, A.K. et al. J. Med. Chem., 52, 5228-40 (2009); Baez-Santos, et al. Antiviral Res., 115, 21-38 (2015); and Zhang, L. et al. Science, 368, 409-412 (2020), and Gorgulla, C. et al. Nature, 580, 663-668 (2020)). The process described above was applied to generate exemplary SP-PROTACs that target and degrade bromodomain-containing proteins (BRD2, BRD3, and BRD4) that bind to the SARS-CoV-2 E protein and the SARS-CoV-2 protein PLpro (FIG. 7).

Additional peptide and molecular modulators of key SARS-CoV-2 targets (PLpro, Mpro, NSP9 and NSP12) may be incorporated into SP-PROTACs using the methods described above. Table 5 lists the SARS-CoV-2 target, the type of the molecule, and the name/structure of the molecule that is incorporated into the SP-PROTAC. Of exemplary interest are SP-PROTACs that incorporate PLpro, Mpro, NSP9 inhibitors and NSP12 inhibitors.

TABLE 5

Examples of small molecules, peptide sequences, and nucleotide analogs that engage viral protein targets, for incorporation into SP-PROTACs

| | | |
|---|---|---|
| Viral Target | Type of Molecule | Molecule |
| PLpro | Small molecule | GRL-0617 |
| Mpro | Small molecule | Ebselen carboxylic acid: 4-(3-oxo-1,2-benzoselenazol-2-yl)benzoic acid |
| Mpro | Peptide (dimerization) | ATVNVLAWLYAAVINGD (SEQ ID NO: 2) |
| Mpro | Stapled Peptide (dimerization) | ATVNVLAWLYX₁AVIX₂GD (SEQ ID NO: 3); wherein X₁ and X₂ = (S)-2-(4′-pentenyl)alanine |
| NSP9 | Stapled Peptide (dimerization) | ANLNAGBX₁LGSX₂AATVELQ (SEQ ID NO: 4); wherein B is norleucine and X₁ and X₂ = (S)-2-(4′-pentenyl)alanine |
| NSP9 | Stapled Peptide (dimerization) | ANLNRGBX₁LGSX₂AATVRLQ (SEQ ID NO: 5), wherein B is norleucine; wherein X₁ and X₂ = (S)-2-(4′-pentenyl)alanine |
| NSP12 | Nucleotide analog | Remdesivir acid ((((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy)phosphoryl)-L-alanine |
| NSP12 | Nucleotide analog | Sofosbuvir Carboxylic Acid (S)-2-(((S)-(((2R,3R,4R,5R)-5-(2,4-Dioxo-3,4-dihydropyrimidin- |
| | | 1(2H)-yl)-4-fluoro-3-hydroxy-4-methyltetrahydrofuran-2-yl)methoxy)(phenoxy)phosphoryl)amino)propanoic Acid |

The SP-PROTACs can also target host proteins such as bromodomain proteins, BRD2, BRD3 and/or BRD4. JQ1 is an exemplary molecule that binds BRD4.

Further, Table 6 below lists examples of E3 ubiquitin ligases and the types of ligand molecules that may bind to these targets. The molecules listed in Table 6 can be attached directly to or linked via a linker to the exemplary molecules of Table 5, to recruit a degrader (e.g., a E3 ligase) for degradation of the viral protein or a host protein that assists viral pathogenesis.

TABLE 6

Examples of ligands which recruit E3 ligases for target degradation

| | | |
|---|---|---|
| E3 ligase | Type of Ligand Molecule | Ligand Molecule |
| HDM2 | Peptides | Stapled p53 peptides (e.g., ATSP-7041, SP645), HDM2-binding variants thereof, and analogs thereof |
| Cereblon | Small molecule | Thalidomide and analogs such as Pomalidomide, Lenalidomide and Avadomide |
| VHL | Small molecule | VH 032 analogs |
| HDM2 | Small molecule | Nutlin (e.g., Nutlin-3a) and analogs such as RG7388 |
| XIAP | Small molecule | A 410099.1 |
| cIAP | Small molecule | SM-1295, SM-1280 |
| COP1 | Peptide | Trib1 |

### Example 2: Size Exclusion Chromatography to Measure Binding Affinity of SP-PROTACs

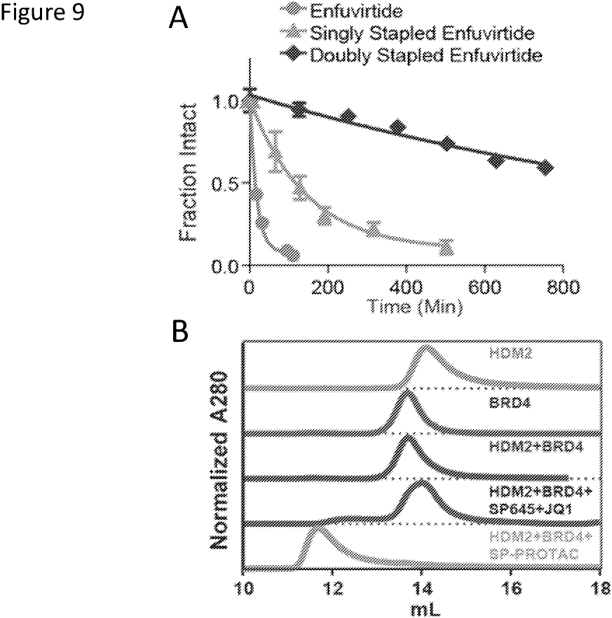

To assess the binding affinities of SP-PROTACs, a qualitative size-exclusion chromatography (SEC) based assay is designed to monitor formation of a ternary complex between the SP-PROTAC, HDM2, and SARS-CoV-2 protein target. Recombinant HDM2 and SARS-CoV-2 proteins (e.g. PLpro, Mpro) bearing an N-terminal hexahistidine tag (SEQ ID NO: 53) and a thrombin cleavage site are cloned into the pET28a vector, expressed in BL21(DE3) E.coli, and purified by affinity Ni-NTA chromatography, followed by tag cleavage and SEC as described (Ben-Nun, Y. et al. Structure 28(7): 847-857 (2020); Bernal, F., et al. J Am Chem Soc 129, 2456-7 (2007)). The components are mixed at a 1:1:1 ratio and the SEC profile is recorded in comparison with each protein alone and the protein combination without added SP-PROTAC. In this manner, the capacity of an SP-PROTAC to form the desired binding complex is documented. As an example of the application of this binding affinity SEC assay, FIG. 9B shows that a BRD4-directed SP-PROTAC was found to induce complex formation between BRD4 and HDM2 (bottom most trace), whereas HDM2 alone, BRD4 alone, HDM2+BRD4, HDM2+BRD4+SP645+JQ1 did not induce complex formation as assessed by SEC. These results confirmed the capacity of SP-PROTACs to nucleate the desired protein complexes in vitro.

### Example 3: Cell Penetrance of SP-PROTACs

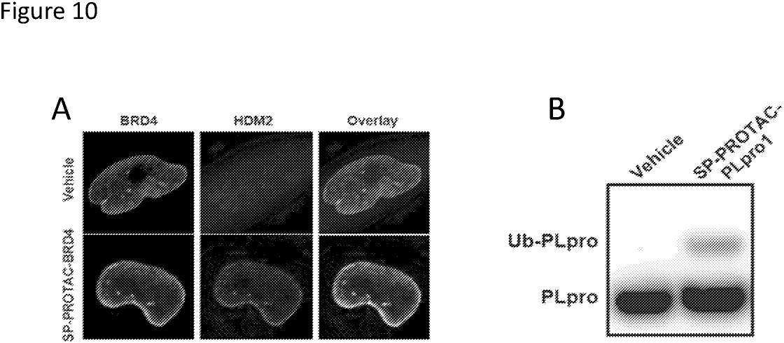

A two-in-one live cell protein interaction assay was used (Herce, H.D. et al. Nat Chem 9, 762-771 (2017)) to simultaneously assess the capacity of SP-PROTACs to enter HeLa cells and generate the desired ternary complex. Briefly, camelid-derived single-chain VHH antibody that recognizes GFP was localized to the nuclear lamina via its laminin fusion. GFP-HDM2 and the target viral protein as an RFP-fusion were then transiently expressed in the cells, followed by treatment with SP-PROTAC or vehicle. A cell penetrant SP-PROTAC that engages both targets was found to concentrate the RFP-viral protein at the nuclear lamina where GFP-HDM2 is anchored, which resulted in protein colocalization, as scored by signal in the GFP/RFP overlay. As shown in FIG. 10A, SP-PROTAC-BRD4 relocalized HDM2 from the cytosol (diffuse pattern, top) to the nuclear lamina (focal pattern, bottom), where BRD4 was experimentally anchored, which resulted in colocalization (bottom right). These results confirmed the capacity of SP-PROTACs to nucleate the desired protein complexes in cellulo (FIG. 10A).

### Example 4: Viral PLpro Protein Ubiquitylation Using SP-PROTACs

To measure the ability of SP-PROTACs to form a ternary complex that enables ubiquitylation of the viral target, in vitro ubiquitylation assays were performed using the HDM2 ubiquitin ligase kit (Boston Biochem) and the corresponding ubiquitin-shift in viral protein molecular weight was monitored by western blot analysis. As shown in FIG. 10B, SP-PROTAC-PLpro1 (10 µM) induced HDM2-mediated ubiquitylation of PLpro in vitro. These result show that inducing HDM2 proximity can achieve viral target (e.g. PLpro) ubiquitylation.

### Example 5: Selective Degradation of Host Protein That Binds to SARS-CoV-2 Protein With SP-PROTAC

To assay for intracellular protein degradation, SJSA-1 cells were passaged at 37° C. in a humidity-controlled, CO₂-equilibrated incubator in DMEM (Life Technologies, Grand Island, NY) culture medium (CM) containing 10% fetal bovine serum (FBS) and 1% penicillin/streptomycin (Pen Strep). One day before treatment, cells were passaged and plated in six well plates at a density of 100,000 cells/mL. After 24 hours, cells were treated with a various concentrations of SP-PROTAC-BRD4 (e.g., 0.1-10 µM) in the presence or absence of carfilzomib (Advanced ChemBlocks Inc, Cat ID: F-4770) 400 nM), a proteasome inhibitor, for 2 h hours after which they were harvested and lysed. Cell lysates were then assayed using standard western blotting techniques using an actin (control), p53, and BRD4 antibodies to assess protein levels, and an actin antibody for the loading control.

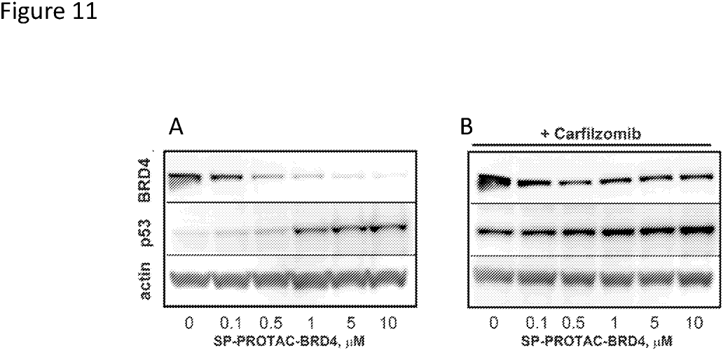

As seen in FIG. 11A, BRD4-directed SP-PROTAC dose-responsively increased p53 and decreased BRD4 levels in SJSA-1 cells. In the presence of a proteasome inhibitor (carfilzomib), BRD4 degradation was inhibited (FIG. 11B).

### Example 6: Change in Protein Landscape of Cultured Cells With SP-PROTAC

The most potent SP-PROTAC compounds from high-content screening were tested in natively-susceptible human-derived Huh7 cells (Mossel, E.C. et al. J Virol 79, 3846-50 (2005)) and Calu-3 cells (Tseng, C.T. et al. J Virol 79, 9470-9 (2005)) that express ACE2. Plated cells were treated for 1 h with a serial dilution of SP-PROTACs (10 µM starting dose, in triplicate) followed by a challenge with SARS-CoV-2 (SARS-CoV-2/human/USA/WA-CDC-WA1/2020; Genbank Accession MN985325.1). Culture supernatants were sampled, virus lysed in the presence of RNAse inhibitor, and reverse transcription (RT) and quantitative polymerase chain reaction (qPCR) performed as described (Suzuki, Y. et al. J Vis Exp (2018)). CDC-validated BHQ quenched dye pair primers were purchased from IDT and genome equivalents calculated from Ct values.

SP-PROTACs that exhibited the most potent anti-viral activity were used to treat Huh7 and Calu-3 cells (>50% infectivity) followed by monitoring for dynamic changes in protein levels. Treated cells were lysed in 10% SDS-containing buffer over time (e.g. 2, 4, 6, 8 hours) and lysates subjected to western analysis for p53, the SARS-CoV-2 target (PLpro, Mpro), and actin control. Lysates from treated cells that manifest on-mechanism p53 induction and target-protein-specific degradation (e.g., PLpro, Mpro, BRD4) also undergo global proteomic analyses, performed as described (Winter, G.E. et al. Science 348, 1376-81 (2015)). In this manner, SP-PROTAC specificity of action across the landscape of cellular proteins was examined for the BRD4-directed SP-PROTAC (FIGS. 12A-C). In contrast to the controls (stapled-peptide SP645 alone (FIG. 12A) and unlinked small molecule JQ1 alone (FIG. 12B)), SP-PROTAC-BRD4 (FIG. 12C) was found to both reduce BRD 2/3/4 protein levels and induce p53 transcriptional targets, such as HDM2. These results show that optimal anti-viral activity derived from the synergistic SP-PROTAC mechanism of p53 induction and target protein degradation.

### Example 7: Improved On-Mechanism Cytotoxicity of Cultured Cells With SP-PROTAC

A cytotoxicity assay was performed using the CellTiter-Glo® Luminescent Cell Viability Assay (Promega, Madison, WI) following manufacturer’s protocols. SJSA-1 cells were treated with the SP645, JQ1, or SP-PROTAC-BRD4 for 72 hours at indicated concentrations In vitro analyses were performed in technical triplicate, repeated at least twice with independent preparations of compounds, proteins, or cells, and analyzed using one-way analysis of variance. Two-tailed p-values <0.05 were considered significant.

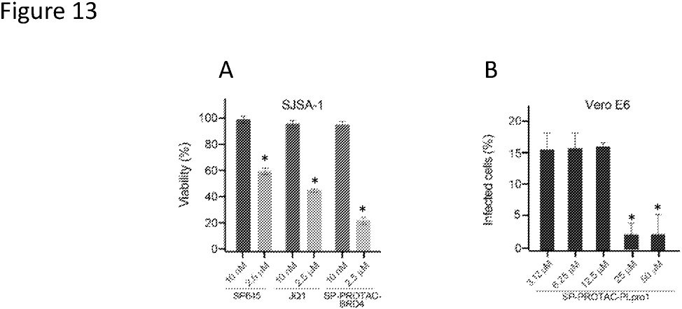

As seen in FIG. 13A, SP-PROTAC-BRD4 significantly improved on-mechanism cytotoxicity (decrease in percent viability) of SJSA-1 cells compared to SP645 and JQ1 alone.

### Example 8: Blockade of Viral Infection of Cultured Cells With SP-PROTAC

A high-throughput viral detection platform has been developed for SARS-CoV-2 based on previous screens against Ebolaviruses (Anantpadma, M. et al. Antimicrob Agents Chemother 60, 4471-81 (2016)). Vero E6 cells plated in 384-well format were treated for 1 h with a serial dilution of SP-PROTACs (50 µM starting dose), performed in triplicate, followed by a 4 h challenge with SARS-CoV-2 (SARS-CoV-2/human/USA/WA-CDC-WA1/2020; Genbank Accession MN985325.1) to achieve control infection of 10-20% cells (optimal infectivity to assess dynamic range of test compounds). Infected cells were then washed, fixed with 4% paraformaldehyde, rewashed in PBS, immune-stained with anti-SARS-CoV-2 nucleocapsid monoclonal antibody (ThermoFisher Sci, Waltham, MA) followed by anti-mouse Ig secondary antibody (Alexa Fluor 488; Life Technologies), and cell bodies counterstained with HCS CellMask blue. Cells were imaged across the z-plane on a Nikon Ti Eclipse automated microscope, analyzed by CellProfiler software, and infection efficiency calculated by dividing infected by total cells. FIG. 13B shows that 25 µM and 50 µM of SP-PROTAC-PLpro1 were found to block SARS-CoV-2 infection of Vero E6 cells

### Example 9: Measurement of α-Helicity of SP-PROTACs

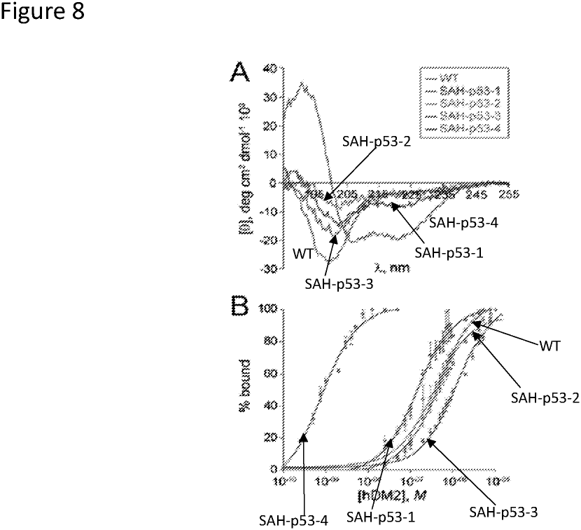

To ensure that small molecule derivatization does not affect the structure of the stapled peptide that is a component of the SP-PROTAC (e.g., SP645, ATSP-7041, etc), the helicity of SP-PROTACs is measured in solution by circular dichroism (CD) (Bird, G.H., et al. Methods Enzymol., 446, 369-86 (2008)). CD spectra are recorded on an Aviv Biomedical spectrometer (Model 410), with compounds reconstituted in 50 µM potassium phosphate (pH 7.5) or Milli-Q deionized water. Five scans from 190-260 nm are averaged to obtain each spectrum, which is plotted as wavelength versus mean residue ellipticity and % helicity calculated as described (Forood, B., et al. Proc Natl Acad Sci USA, 90, 838-42 (1993)). As an example of the above method, the helicity of SP-PROTACs was assessed by circular dichroism for four HDM2-binding stapled p53 peptides stapled peptides (SAH-p53-1 to -4; SEQ ID NOs.: 40-43) as shown in Bernal F., et al., J. Am. Chem. Soc. 129(9):2456-2457 (2007).). FIG. 8A (identical to FIG. 1C in Bernal F., et al.) shows that SAH-p53-4 (SEQ ID NO: 43) was more stable than SAH-p53-1-3 (SEQ ID NOs.: 40-42) or the wild type p53 peptide (SEQ ID NO: 11).

### Example 10: Measurement of Proteolytic Stability of SP-PROTACs

A key feature of stapled peptides is their capacity to resist proteolysis in vivo due to shielding of the labile amide bonds by the staple itself and induced helical-folding. To determine proteolytic stability, comparative proteolytic analyses is performed by Liquid chromatography-mass spectrometry (LC-MS) (Agilent 1200) as described (Bird, G.H. et al. ACS Chem Biol, 15, 6, 1340-1348 (2020)) to identify the most protease-resistant compounds for cellular and in vivo studies. Reaction samples are composed of 5 µl SP-PROTAC in DMSO (1 mM stock) and 195 µl buffer consisting of 50 mM Tris HCl, pH 7.4. Upon injection of the zero-time sample, 2.5 µl of 100 ng/µL proteinase K (New England Biolabs) is added, and the amount of intact compound quantitated by serial injection over time. A plot of mean square displacement (MSD) area versus time yields an exponential decay curve, and half-lives are determined by nonlinear regression analysis using Prism software (GraphPad). An example of the application of this proteolytic stability assessment is shown in Bird G.H., et al., PNAS Aug. 10, 2010 107 (32) 14093-14098.. FIG. 9A (derived from Bird G.H., et al.) shows that singly stapled and doubly stapled anti-HIV therapeutic enfuvirtide were found to have striking protease resistance compared to unstapled enfuvirtide. Stability of enfuvirtide increased proportionally with the number of staples.

### Example 11: Size Exclusion Chromatography to Measure Binding Affinity of SP-PROTACs

To evaluate the relative binding affinity for each component of the SP-PROTACs, fluorescence polarization (FP) binding analyses are performed in which C-terminally FITC-derivatized SP-PROTAC (e.g. 25 nM) with a serial dilution of each protein individually (e.g. HDM2, PLpro) in binding buffer (50 mM NaCl, 20 mM HEPES pH 7.4, 5 mM DTT) is incubated. FP is measured at equilibrium on a Spectramax M5 Microplate Reader (Molecular Devices) and the data plotted and Kd values calculated using Prism software (Graphpad). An example of the application of this FP analysis is shown in Bernal F., et al., J. Am. Chem. Soc. 129(9):2456-2457 (2007). FIG. 8B (identical to FIG. 1D in Bernal F. et al.) shows the relative binding affinity of various stapled p53 peptides (SEQ ID NOs.: 40-43) to HDM2. SAH-p53-4 was found to bind with higher affinity to HDM2 compared with SAH-p53-1-3 or the wild type p53 peptide.

### Example 12: Anti-Viral Activity of Lead SP-PROTACs in the Humanized ACE2 Receptor Mouse Model of SARS-CoV-2 Infection

In vivo efficacy testing of SP-PROTACs is performed in transgenic mice expressing human ACE2 receptor in airway epithelium (McCray, P.B., Jr. et al. J Virol 81, 813-21 (2007); Yan, R. et al. Science (2020)) and infected with SARS-CoV-2 virus (Netland, J., et al. J Virol 82, 7264-75 (2008); Tseng, C.T. et al. J Virol 81, 1162-73 (2007)). Experiments are conducted using the methods developed for evaluation of anti-Ebolavirus therapeutics (Pascal, K.E. et al. J Infect Dis 218, S612-s626 (2018); Sakurai, Y. et al. Science 347, 995-8 (2015)). SP-PROTACs are first tested for tolerance. For each of 3 treatments, 20 mice in groups of 10 (5 male, 5 female) are dosed intraperitoneally twice daily for 10 days with high or low dose SP-PROTAC. Another group of 10 mice receive saline only as a control. Animals are examined before each dose and treatment discontinued if adverse effects are observed. If after 10 days of dosing, the high dose is not tolerated, a lower dose is used. Tolerated SP-PROTACs are tested for efficacy in the hACE2 mouse disease model. K18-hACE2 mice (JAX; n=20 per arm, 10 male, 10 female) are inoculated intranasally at a viral dosage of 10⁴ PFU on Day 1, followed by daily intraperitoneal treatment with SP-PROTAC (dosing amount and frequency based on tolerance and PK results) for 10 days (Days 2-12). One group receives vehicle as control. Mice are continuously monitored to record body weights and clinical signs. On day 4 (peak of viremia), 4 mice from each group are euthanized and viral load quantitated by qPCR from lung homogenate supernatants, prepared as described (Bao, L. et al. Nature 583, 830-833 (2020) using a tissuelyzer (Qiagen). Doses for the most effective treatment are refined to determine the minimum amount to protect mice. The same experimental design is used but the 3 groups receive lower doses of the treatment in 4-fold increments. Each experiment addresses sex as a biological variable with equal numbers of male and female mice.

For in vivo tolerance, 10 mice/dose/compound and 10 mice for saline control are used, and mice are euthanized at day 10 (or earlier for adverse signs) and examined for toxicity. The studies have 80% power to detect increased toxicity (60% v 10%), p=0.10. For in vivo efficacy, disease progression is defined as >10% weight reduction, labored breathing, or failure to thrive, and animals treated with SP-PROTAC vs. vehicle are compared. 16 animals (plus 4 mice for evaluation on day 4) per group will result in 81% power to detect 70% of treated vs. 22% of control mice that are progression-free, p=0.05. Time to progression is evaluated using the method of Kaplan and Meier and test for differences using the log rank test.

### Example 13: Viral NSP9 Protein Ubiquitylation Using SP-PROTACs

To measure the ability of alternatively designed SP-PROTACs to form a ternary complex with another SARS-CoV-2 viral protein, we generated an SP-PROTAC comprised of either (1) an i, i+4 single-stapled NSP9 binding peptide (SEQ ID NO: 4) positioned at the N-terminus and linked to SP-645 (SEQ ID NO: 1), named SP-PROTAC-NSP9-1 (SEQ ID NO: 54), or (2) SP-645 (SEQ ID NO: 1) positioned a the N-terminus and linked to an i, i+4 single-stapled NSP9 binding peptide (SEQ ID NO: 4), named SP-PROTAC-NSP9-2 (SEQ ID NO: 55; FIG. 14A). In both cases, the SP-PROTAC successfully nucleated the ternary complex between recombinant HDM2, SP-PROTAC-NSP9, and recombinant NSP9 to enable HDM2 ubiquitylation of the viral target protein, NSP9. In vitro ubiquitylation assays were performed using the HDM2 ubiquitin ligase kit (Boston Biochem) and the corresponding ubiquitin-shifts in viral protein molecular weight were monitored by western blot analysis. As shown in FIG. 14B, SP-PROTAC-NSP9-1 and -2 (10 µM) induced HDM2-mediated ubiquitylation of NSP9 in vitro. These results show that inducing HDM2 proximity can achieve targeted viral protein (e.g. NSP9) ubiquitylation for degradation.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A chimera, comprising:
a first moiety attached to a second moiety, wherein the first moiety and second moiety are directly attached to each other or attached to each other via a linker;
wherein the first moiety binds to a first protein targeted for degradation, wherein the first protein is selected from a coronaviral protease, a coronaviral non-structural protein (NSP), or a bromodomain and extraterminal domain (BET) protein; and
the second moiety binds to a second protein, wherein the second protein is, or recruits, a protein degrader.

2. The chimera of claim 1, wherein the second protein is an E3 ubiquitin ligase.

3. The chimera of claim 1, wherein the first moiety and the second moiety are attached to each other via a linker, optionally wherein the linker is a peptide linker, a chemical linker, a Glycine-Serine linker, (G4S)₃ (SEQ ID NO: 26), (G4S)₅ (SEQ ID NO: 27), a beta-alanine (Z) linker, a beta-alanine and alanine (ZA) linker, or a polyethylene glycol linker.

4. The chimera of any one of claims 1-3, wherein the first moiety comprises a small molecule, a small molecule derivatized with a warhead, a peptide, a stapled peptide, a peptide derivatized with a warhead, a stapled peptide derivatized with a warhead, or a nucleotide analog.

5. The chimera of any one of claims 1-4, wherein the coronaviral protease is papain-like protease (PLpro), or main protease (Mpro); the coronaviral NSP is NSP9 or NSP12; and the BET protein is bromodomain 2 (BRD2), bromodomain 3 (BRD3), or bromodomain 4 (BRD4).

6. The chimera of claim 5, wherein the coronaviral protease is PLpro and the first moiety binds to PLpro.

7. The chimera of claim 6, wherein the first moiety that binds to PLpro is a PLpro inhibitor.

8. The chimera of claim 7, wherein the PLpro inhibitor is GRL-0617 or a PLpro-binding analog thereof, disulfiram, or a PLpro-binding thiopurine analog.

9. The chimera of claim 5, wherein the coronaviral protease is Mpro and the first moiety binds to Mpro.

10. The chimera of claim 9, wherein the first moiety that binds to Mpro is an Mpro inhibitor.

11. The chimera of claim 10, wherein the Mpro inhibitor is Lopinavir, Ritonavir, Darunavir, ASC09, GC376, GC813, Ebselen carboxylic acid, or a peptide comprising an amino acid sequence with at least 30% identity to the sequence set forth in SEQ ID NO: 2, or SEQ ID NO: 3, wherein the peptide binds Mpro.

12. The chimera of claim 5, wherein the BET protein is BRD4 and the first moiety binds to the BET protein.

13. The chimera of claim 12, wherein the first moiety that binds to the BET protein is a BET protein inhibitor.

14. The chimera of claim 13, wherein the BET protein inhibitor is JQ1, ABBV-075, I-BET151, I-BET726, OTX015, or PFI-1, or analogs thereof that bind BRD4, BRD3 and/or BRD2.

15. The chimera of claim 5, wherein the coronaviral NSP is NSP9 and the first moiety binds to NSP9.

16. The chimera of claim 15, wherein the first moiety that binds to NSP9 is an NSP9 inhibitor.

17. The chimera of claim 16, wherein the first moiety is a peptide comprising an amino acid sequence with at least 30% identity to the sequence set forth in SEQ ID NO: 4 or SEQ ID NO: 5, wherein the peptide binds NSP9.

18. The chimera of claim 5, wherein the coronaviral NSP is NSP12 and the first moiety binds to NSP12.

19. The chimera of claim 18, wherein the first moiety that binds to NSP12 is an NSP12 inhibitor.

20. The chimera of claim 19, wherein the first moiety is remdesivir acid or an analog thereof that binds NSP12, or sofosbuvir acid or an analog thereof that binds NSP12.

21. The chimera of any one of claims 1-20, wherein the second protein is human double minute 2 (HDM2), Von Hippel-Lindau (VHL), Cereblon, X-linked inhibitor of apoptosis protein (XIAP), cellular inhibitor of apoptosis protein (cIAP), or Constitutive photomorphogenic 1 (COP1).

22. The chimera of any one of claims 1-21, wherein the second moiety comprises a peptide, a stapled peptide, or a small molecule that binds to or recruits the protein degrader.

23. The chimera of any one of claims 1-22, wherein the second moiety comprises a cereblon binding moiety that is a small molecule, optionally selected from a group consisting of thalidomide, pomalidomide, lenalidomide, avadomide, and analogs thereof that bind cereblon.

24. The chimera of claim 23, wherein the second moiety comprises a thalidomide moiety.

25. The chimera of claim 24, wherein the thalidomide moiety comprises the structure provided below: 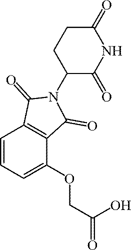 [SMILES: O=C(O)COc1cccc2c1C(=O)N(C1CCC(=O)NC1=O)C2=O]
or a cereblon-binding analog thereof.

26. The chimera of claim 24, wherein the thalidomide moiety comprises the structure provided below: 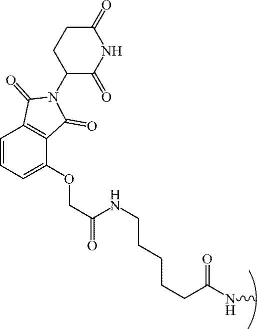
or a cereblon-binding analog thereof.

27. The chimera of claim 24, wherein the thalidomide moiety comprises the structure provided below: 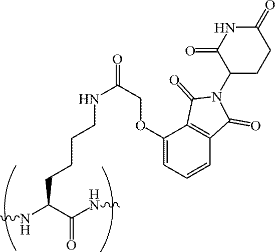
or a cereblon-binding analog thereof.

28. The chimera of any one of claims 1-22, wherein the second moiety comprises a VHL binding moiety, optionally selected from a group consisting of VH 032 and VHL-binding analogs thereof.

29. The chimera of claim 28, wherein the VHL binding moiety comprises the structure below: 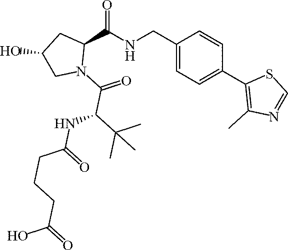 [SMILES: Cc1ncsc1-c1ccc(CNC(=O)[C@@H]2C[C@@H](O)CN2C(=O)[C@@H](NC(=O)CCCC(=O)O)C(C)(C)C)cc1]
or a VHL-binding analog thereof.

30. The chimera of claim 28, wherein the VHL binding moiety comprises the structure below: 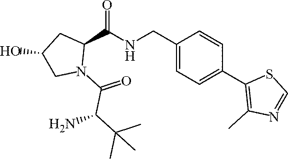 [SMILES: Cc1ncsc1-c1ccc(CNC(=O)[C@@H]2C[C@@H](O)CN2C(=O)[C@@H](N)C(C)(C)C)cc1]
or a VHL-binding analog thereof.

31. The chimera of any one of claims 1-22, wherein the second moiety comprises an HDM2 binding moiety.

32. The chimera of claim 31, wherein the HDM2 binding moiety comprises a peptide or a stapled peptide or an otherwise chemically-stabilized peptide of the transactivation domain of p53 that binds HDM2 and/or HDMX.

33. The chimera of claim 32, wherein the HDM2 binding moiety is a stapled peptide that is ATSP-7041, SP645, or an HDM2-binding variant thereof.

34. The chimera of claim 33, wherein the stapled peptide comprises the sequence LTF(R8)EYWAQ#(S5)SAA (SEQ ID NO: 7), or a peptide comprising
(a) an amino acid sequence with at least 30% identity to the sequence set forth in SEQ ID NO: 7, or
(b) an amino acid sequence with at least 30% identity to the sequence set forth in SEQ ID NO: 7, wherein the amino acids on the interacting face of the peptide are not substituted; or (c) an amino acid sequence with at least at least 30% identity to the sequence set forth in SEQ ID NO: 7, wherein one or more of the amino acids on the interacting face of the peptide are substituted with a conservative amino acid; wherein (R8) is (R)-2-(7′-octenyl)alanine, # is cyclobutylalanine, and (S5) is (S)-2-(4′-pentenyl)alanine or a HDM2-binding variant thereof;
wherein the peptide binds HDM2.

35. The chimera of claim 33, wherein the stapled peptide comprises the sequence LTF(R8)EYWAQL(S5)SAA (SEQ ID NO: 1), or an HDM2-binding variant thereof, or a peptide comprising
(a) an amino acid sequence with at least 30% identity to the sequence set forth in SEQ ID NO: 1, or
(b) an amino acid sequence with at least 30% identity to the sequence set forth in SEQ ID NO: 1, wherein the amino acids on the interacting face of the peptide are not substituted; or
(c) an amino acid sequence with at least at least 30% identity to the sequence set forth in SEQ ID NO: 1, wherein one or more of the amino acids on the interacting face of the peptide are substituted with a conservative amino acid;
wherein (R8) is (R)-2-(7′-octenyl)alanine and (S5) is (S)-2-(4′-pentenyl)alanine, and
wherein the peptide binds HDM2.

36. The chimera of claim 31, wherein the HDM2 binding moiety is Nutlin-3a or an HDM2-binding analog thereof.

37. The chimera of claim 36, wherein the HDM2 binding moiety comprises the structure below: 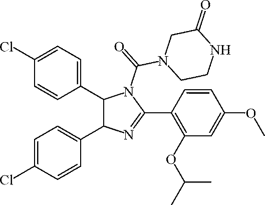 [SMILES: COc1ccc(C2=NC(c3ccc(Cl)cc3)C(c3ccc(Cl)cc3)N2C(=O)N2CCNC(=O)C2)c(OC(C)C)c1]
.

38. The chimera of any one of claims 1-21, wherein the second moiety comprises an XIAP binding moiety that is A410099.1 or an XIAP-binding analog thereof.

39. The chimera of claim 38, wherein the XIAP binding moiety comprises the structure below: 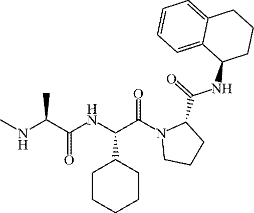 [SMILES: CN[C@@H](C)C(=O)N[C@H](C(=O)N1CCC[C@H]1C(=O)N[C@@H]1CCCc2ccccc21)C1CCCCC1]
.

40. The chimera of any one of claims 1-21, wherein the second moiety comprises cIAP binding moiety that is SM-1295, SM-1280 or a cIAP-binding analog thereof.

41. The chimera of any one of claims 1-40, wherein the second moiety comprises a peptide that binds a WD40-repeat protein that is a substrate adaptor for an E3 ubiquitin ligase, wherein the peptide comprises a modified version of a natural binding sequence or a natural binding consensus sequence of an amino acid sequence that binds to the WD40-repeat protein, wherein the modified version comprises at least one amino acid substitution, at least one amino acid deletion, at least one amino acid insertion, or any combination thereof within the natural binding consensus sequence.

42. The chimera of claim 41, wherein the WD40-repeat protein is a substrate adaptor for the E3 ubiquitin ligase HDM2 or VHL.

43. The chimera of claim 41, wherein the natural binding consensus sequence SEQ ID NOs.: 14 or 15, or a variant thereof, wherein the variant differs from the consensus sequence at one to six amino acid positions.

44. The chimera of any one of claims 1-21, wherein the second moiety comprises a COP1 binding moiety.

45. The chimera of claim 44, wherein the COP1 binding moiety is a peptide, that is a Tribbles Pseudokinase 1 (Trib1) peptide or a COP1-binding variant thereof.

46. The chimera of claim 45, wherein the peptide comprises the sequence DQIVPEY (SEQ ID NO: 6), or a peptide comprising an amino acid sequence with at least 30% identity to the sequence set forth in SEQ ID NO: 6.

47. The chimera of any one of claims 1-46, wherein the protein degrader degrades the first protein.

48. A pharmaceutical composition comprising the chimera of any one of clams 1-47 and a pharmaceutically acceptable carrier.

49. The pharmaceutical composition of claim 48, wherein the pharmaceutical composition is formulated for oral, intravenous, topical, buccal, rectal, parenteral, intraperitoneal, intradermal, subcutaneous, intramuscular, transdermal, intranasal, pulmonary, or intratracheal administration.

50. A method of treating or preventing a viral infection caused by a coronavirus in a subject in need thereof, the method comprising administering to the subject a therapeutically effective amount of the chimera of any one of claims 1-45, or the pharmaceutical composition of claim 47 or 48.

51. A method for both blocking viral replication and reducing viral infectivity of coronavirus in a subject in need thereof, the method comprising administering to the subject a therapeutically effective amount of the chimera of any one of claims 1-47, or the pharmaceutical composition of claim 48 or 49.

52. The method of claim 50 or 51, wherein the coronavirus is Middle East Respiratory Syndrome coronavirus (MERS-CoV), severe acute respiratory syndrome coronavirus (SARS-CoV), or SARS-CoV-2.

53. A method for blocking the replication of SARS-CoV or SARS-CoV-2 in a subject in need thereof, the method comprising administering to the subject a therapeutically effective amount of the chimera of any one of claims 1-47, or the pharmaceutical composition of claim 48 or 49.

54. A method for treating or preventing an RNA virus infection in a subject in need thereof, the method comprising administering to the subject a therapeutically effective amount of the chimera of any one of claims 1-45, or the pharmaceutical composition of claim 48 or 49.

55. The method of any one of claims 50-54, further comprising administering to the subject one or more agents selected from the group consisting of a corticosteroid, hydrocortisone, methylprednisolone, dexamethasone, remdesivir, an IL-6 inhibitor, an IL-1 inhibitor, a kinase inhibitor, a complement inhibitor, ivermectin, hydroxychloroquine, favipiravir, interferon-beta, and icatibant.

56. The method of any one of claims 50-55, wherein the subject is selected from a group consisting of a human, a primate, a bat, a bird, a mouse, a turkey, a cow, a pig, a cat and a dog.

57. A peptide comprising the amino acid sequence set forth in SEQ ID NOs.: 2 or 3, or a variant thereof, wherein the peptide binds and inhibits Mpro.

58. A stabilized peptide comprising a peptide comprising the amino acid sequence set forth in SEQ ID NOs.: 2 or 3 with 1, 2, 3, 4, 5, or 6 amino acid substitutions, wherein at least two amino acid substitutions replace amino acids separated by three or six amino acids with non-natural amino acids, and wherein the peptide binds and inhibits Mpro.

59. The peptide or stabilized peptide of claims 57 or 58, which is less than 50, 40, 35, 30, or 25 amino acids in length.

60. A peptide comprising the amino acid sequence set forth in SEQ ID NOs.: 4 or 5, or a variant thereof, wherein the peptide binds NSP9.

61. A stabilized peptide comprising a peptide comprising the amino acid sequence set forth in SEQ ID NOs.: 4 or 5 with 1, 2, 3, 4, 5, or 6 amino acid substitutions, wherein at least two amino acid substitutions replace amino acids separated by three or six amino acids with non-natural amino acids, and wherein the peptide binds NSP9.

62. The peptide or stabilized peptide of claims 60 or 61, which is less than 50, 40, 35, 30, or 25 amino acids in length.

63. A pharmaceutical composition comprising peptide or stabilized peptide of any one of claims 58-62, and a pharmaceutically acceptable carrier.

64. A method of treating or preventing a coronaviral infection in a subject in need thereof, the method comprising administering to the subject a therapeutically effective amount of the peptide or stabilized peptide of any one of claims 58-62, or the pharmaceutical composition of claim 63.

65. A chimera comprising a compound having the structure of any one of the molecules depicted in FIG. 7 or FIG. 14A.

66. A pharmaceutical composition comprising the chimera of claim 65 and a pharmaceutically acceptable carrier.

67. A method of treating or preventing a viral infection caused by a coronavirus in a subject in need thereof, the method comprising administering to the subject a therapeutically effective amount of the chimera of claim 65, or the pharmaceutical composition of claim 66.

68. A method for both blocking viral replication and reducing viral infectivity of coronavirus in a subject in need thereof, the method comprising administering to the subject a therapeutically effective amount of the chimera of claim 65, or the pharmaceutical composition of claim 66.

69. A method for blocking the replication of SARS-CoV or SARS-CoV-2 in a subject in need thereof, the method comprising administering to the subject a therapeutically effective amount of the chimera of claim 65, or the pharmaceutical composition of claim 66.

70. A method for treating or preventing an RNA virus infection in a subject in need thereof, the method comprising administering to the subject a therapeutically effective amount of the chimera of claim 65, or the pharmaceutical composition of claim 66.

71. The method of any one of claims 67-70, wherein the subject is a human.
